SEQUENCE LISTING (511 sequences)

SEQ ID NO: 1 - DNA, 80 bp, Homo sapiens
  variation (39)..(39): N = T or no nucleotide
aaaggctgaattctgtaataaaagcaaacagcctggctnagcaaggagccaacataacagatgggctggaagtaaggaaa

SEQ ID NO: 2 - DNA, 80 bp, Homo sapiens
  variation (43)..(43): N = T or C
gaagaccccaaagatctcatgttaagtggagaaagggttttgnaaactgaaagatctgtagagagtagcagtatttcatt

SEQ ID NO: 3 - DNA, 61 bp, Homo sapiens
  variation (31)..(31): N = A or C
cagatgcaaacagctataattttgcaaaaanggaaaataactctcctgaacatctaaaaga

SEQ ID NO: 4 - DNA, 61 bp, Homo sapiens
  variation (31)..(31): N = T or C
agctataattttgcaaaaaaggaaaataacnctcctgaacatctaaaagatgaagtttcta

SEQ ID NO: 5 - DNA, 61 bp, Homo sapiens
  variation (31)..(31): N = G or no nucleotide
ccccaattgaaagttgcagaatctgcccagnagtccagctgctgctcatactactgatact

SEQ ID NO: 6 - DNA, 100 bp, Homo sapiens
  variation (40)..(40): N = T or no nucleotide
agcggatacaacctcaaaagacgtctgtctcattgaattngggtaagggtctcaggttttttaagtatttaataataattgctggattccttatcttata

SEQ ID NO: 7 - DNA, 121 bp, Homo sapiens
  variation (61)..(61): N = T or C
ctctgcttgtgttctctgtctccagcaattgggcagatgtgtgaggcacctgtggtgaccngagagtgggtgttggacagtgtagcactctaccagtgccaggagctggacacctacctga

SEQ ID NO: 8 - DNA, 121 bp, Homo sapiens
  variation (61)..(61): N = T or C
aaaggagagcttagcaggagtcctagccctttcacccatacacatttggctcagggttacngaagaggggccaagaaattagagtcctcagaagagaacttatctagtgaggatgaagagc

SEQ ID NO: 9 - DNA, 121 bp, Homo sapiens
  variation (61)..(61): N = A or C
actgaaagatctgtagagagtagcagtatttcattggtacctggtactgattatggcactnaggaaagtatctcgttactggaagttagcactctagggaaggcaaaaacagaaccaaata

SEQ ID NO: 10 - DNA, 121 bp, Homo sapiens
  variation (61)..(61): N = T or C
gaaacaagcgtctctgaagactgctcagggctatcctctcagagtgacattttaaccactnaggtaaaaagcgtgtgtgtgtgtgcacatgcgtgtgtgtggtgtcctttgcattcagtag

SEQ ID NO: 11 - DNA, 121 bp, Homo sapiens
  variation (61)..(61): N = A or C
ggtggcgatggttttctccttccatttatctttctaggtcatccccttctaaatgcccatnattagatgataggtggtacatgcacagttgctctgggagtcttcagaatagaaactaccc

SEQ ID NO: 12 - DNA, 121 bp, Homo sapiens
  variation (61)..(61): N = AG or no nucleotide
tttatctgctcttcgcgttgaagaagtacaaaatgtcattaatgctatgcagaaaatcttnagtgtcccatctggtaagtcagcacaagagtgtattaatttgggattcctatgattatct

SEQ ID NO: 13 - DNA, 121 bp, Homo sapiens
  variation (61)..(61): N = C or no nucleotide
atttaatttcagatgctcgtgtacaagtttgccagaaaacaccacatcactttaactaatntaattactgaagagactactcatgttgttatgaaaacaggtataccaagaacctttacag

SEQ ID NO: 14 - DNA, 121 bp, Homo sapiens
  variation (61)..(61): N = C or no nucleotide
aagtcagaggagatgtggtcaatggaagaaaccaccaaggtccaaagcgagcaagagaatncccaggacagaaaggtaaagctccctccctcaagttgacaaaaatctcaccccaccactc

SEQ ID NO: 15 - DNA, 121 bp, Homo sapiens
  variation (61)..(61): N = A or G
accacatcactttaactaatctaattactgaagagactactcatgttgttatgaaaacagntataccaagaacctttacagaataccttgcatctgctgcataaaaccacatgaggcgagg

SEQ ID NO: 16 - DNA, 121 bp, Homo sapiens
  variation (61)..(61): N = A or G
caagtttgaggtgtctgcagatagttctaccagtaaaaataaagaaccaggagtggaaagntaagaaacatcaatgtaaagatgctgtggtatctgacatctttatttatattgaactctg

SEQ ID NO: 17 - DNA, 61 bp, Homo sapiens
  variation (35)..(35): N = GT or no nucleotide
tttaagatagaaaatcataatgataaaactgtaangaaaaaaataataaatgccaactgat

SEQ ID NO: 18 - DNA, 61 bp, Homo sapiens
  variation (31)..(31): N = AGTT or no nucleotide
aaagtttctaaaatatcaccttgtgatgttntggaaacttcagatatatgtaaatgtagta

SEQ ID NO: 19 - DNA, 61 bp, Homo sapiens
  variation (31)..(31): N = A or G
tgcatatactgcatgcaaatgatcccaagtngtccaccccaactaaagactgtacttcagg

SEQ ID NO: 20 - DNA, 51 bp, Homo sapiens
  variation (26)..(26): N = A or C
agggtctccttcgcagtccaagaacnctactgctattcactcttcagatgt

SEQ ID NO: 21 - DNA, 61 bp, Homo sapiens
  variation (24)..(24): N = C or G
cctgaggagcctacccctgccccntgggctcgattatgggcccttcaggatggatttgcca

SEQ ID NO: 22 - DNA, 51 bp, Homo sapiens
  variation (26)..(26): N = C or G
acttttccccctagttgtgtcttgcnatgctaaaggacgtcacattgcaca

SEQ ID NO: 23 - DNA, 61 bp, Homo sapiens
  variation (31)..(31): N = CA or no nucleotide
gtccgcgccatggccatctacaagcagtcangcacatgacggaggttgtgaggcgctgccc

SEQ ID NO: 24 - DNA, 61 bp, Homo sapiens
  variation (31)..(31): N = G or C
aagctcccagaatgccagaggctgctccccncgtggcccctgcaccagcagctcctacacc

SEQ ID NO: 25 - DNA, 51 bp, Homo sapiens
  variation (26)..(26): N = C or G
cgcgcgggcgtgcgagcagcgaaagngacaggggcaaagtgagtgacctgc

SEQ ID NO: 26 - DNA, 51 bp, Homo sapiens
  variation (26)..(26): N = C or T
gcattcccgggcgggtgacccagcanggtccctcttggaattggattcgcc

SEQ ID NO: 27 - DNA, 51 bp, Homo sapiens
  variation (26)..(26): N = A or C
gccagctgtaggccagaccctggcangatctgggtggataatcagactgac

SEQ ID NO: 28 - DNA, 51 bp, Homo sapiens
  variation (26)..(26): N = C or T
gagtgtgtgcgtgtggggttgagggngttggagcggggagaaggccagggg

SEQ ID NO: 29 - DNA, 121 bp, Homo sapiens
  variation (61)..(61): N = A or G
ctttttttttaaattaaagtaacatttccaatctactaatgctaatactgtttcgtatttntagctgatttgatggagttggacatggccatggaaccagacagaaaagcggctgttagtc

SEQ ID NO: 30 - DNA, 121 bp, Homo sapiens
  variation (61)..(61): N = T or G
ttccatgaatccagggcctaacatgggacagccgcaaccaggaatgacttctagtaagtgntttttgttatatttctgtttgagagaaattgataataaaatagtttctatctaaagtcat

SEQ ID NO: 31 - DNA, 121 bp, Homo sapiens
  variation (61)..(61): N = G or no nucleotide
agcacatcgagagcaagctcctgggctccaattcctccaggatgtacttcacccaggtcangggcgcttctcatccagctacttctctggggcctttgaaatgtgcccggccagacgtgag

SEQ ID NO: 32 - DNA, 121 bp, Homo sapiens
  variation (61)..(61): N = T or C
acaatccatttattagtagcagaaagaagtttaaaatcttgctttctgatataatttgttntgtaggccccaatatgggaggtaaatcaacatatattcgacaaactggggtgatagtact

SEQ ID NO: 33 - DNA, 121 bp, Homo sapiens
  variation (61)..(61): N = T or C
gataaacagatgttccacatcattactggccccaatatgggaggtaaatcaacatatattngacaaactggggtgatagtactcatggcccaaattgggtgttttgtgccatgtgagtcag

SEQ ID NO: 34 - DNA, 121 bp, Homo sapiens
  variation (61)..(61): N = A or G
aaagtcactaattaaacttcattttcatgtatcttatgctatttcataaaaaatatttctnttttcagtgttctacccaagagttcttcttgattgtcaaaactttatatcacctaaagtc

SEQ ID NO: 35 - DNA, 121 bp, Homo sapiens
  variation (61)..(61): N = T or C
tgtcttacattatggttttccaaattttgatttgtttttaaatactctttccttgcctggnaggtaggcacaacttacgtaacagataagagtgaagaagataatgaaattgagagtgaag

SEQ ID NO: 36 - DNA, 121 bp, Homo sapiens
  variation (61)..(61): N = A or G
gacaacttacggttttcgtggagaagccttggggtcaatttgttgtatagctgaggtaagntaattatattggttattttagtgatttcatattcactgaacattacagttaaaactaacc

SEQ ID NO: 37 - DNA, 121 bp, Homo sapiens
  variation (61)..(61): N = A or G
ctcagtgtctggctggctgtccaacctctccaggaagctgacatacctacctcagcagggncctgaggaacagactcatctttccacgtctcacccagggatggagccaggtcctcagaag

SEQ ID NO: 38 - DNA, 121 bp, Homo sapiens
  variation (61)..(61): N = A or G
gccttaccttggaagtggacgtaggtgttgaaagccagggtgctgtccacactggctcccntcagggagcagccagtcttccatcctgtcacagcctgcatgaacctgtcaatcttctcag

SEQ ID NO: 39 - DNA, 61 bp, Homo sapiens
  variation (31)..(31): N = T or C
ctggagatagaactgaagcaggtcatgcagnagcgctctgaggacaatgcccggcacaagc

SEQ ID NO: 40 - DNA, 61 bp, Homo sapiens
  variation (31)..(31): N = GC or no nucleotide
ggagatggccgagatgcgggcaaggatgcanagcagctggacgagtaccaggagcttctgg

SEQ ID NO: 41 - DNA, 61 bp, Homo sapiens
  variation (31)..(31): N = A or G
gatgccccagaggacacaggtgacagcgatnagtgggtgtttgacaagaaggtgagtgaga

SEQ ID NO: 42 - DNA, 61 bp, Homo sapiens
  variation (31)..(31): N = A or G
tctacacggtcacagtgaagaaccctgtggncgaggaccaggtcaacctcacagtcaaggt

SEQ ID NO: 43 - DNA, 61 bp, Homo sapiens
  variation (31)..(31): N = A or G
cggagccggtgacagtgcaggagatcctgcntgagtgcccctttgtgcagtcacaagaccc

SEQ ID NO: 44 - DNA, 61 bp, Homo sapiens
  variation (31)..(31): N = A or C
cgtcttggagcattaccgccgcacccactgngtggtgccagagctcatcattggcaatggc

SEQ ID NO: 45 - DNA, 61 bp, Homo sapiens
  variation (31)..(31): N = A or G
ctttaccttatttatagcccaagatttcctngttcaagaatggcctggacctgggagaaga

SEQ ID NO: 46 - DNA, 61 bp, Homo sapiens
  variation (31)..(31): N = T or C
agcccaagatttcctggttcaagaatggccnggacctgggagaagacgcccgcttccgcat

SEQ ID NO: 47 - DNA, 61 bp, Homo sapiens
  variation (31)..(31): N = C or no nucleotide
tggtcctacccctggagcccccgatgacccnattggcctcttcgtgatgcggccacaggat

SEQ ID NO: 48 - DNA, 61 bp, Homo sapiens
  variation (31)..(31): N = A or G
cctggacctcctatcagccttccgccgcacntgagtggccatcctcagggcctgggggagg

SEQ ID NO: 49 - DNA, 145 bp, Homo sapiens
  variation (61)..(61): N = A
tgggaccccccgagtagaaacacagatgtgtctccctgggtccctgccaggtcccctctcngcctggatggcttccctccctctctttaccttatttatagcccaagatttcctggttcaagaatggcctggacctgggagaaga

SEQ ID NO: 50 - DNA, 61 bp, Homo sapiens
  variation (31)..(31): N = A or G
aagcagcgggaggagcaggcggagccagacngcactgaaggtgggaggcagggattcttgg

SEQ ID NO: 51 - DNA, 61 bp, Homo sapiens
  variation (31)..(31): N = A or G
aaaatctgaacaagctgatgaccaacttgcnctccacccatccccactttgtacgttgtat

SEQ ID NO: 52 - DNA, 61 bp, Homo sapiens
  variation (31)..(31): N = A or G
accgcatcctctacggggacttccggcagangtgggtatgagggtgcaccagagctcatag

SEQ ID NO: 53 - DNA, 61 bp, Homo sapiens
  variation (31)..(31): N = A or G
gggacgagaggctgagccgcatcatcacgcntatccaggcccagtcccgaggtgtgctcgc

SEQ ID NO: 54 - DNA, 61 bp, Homo sapiens
  variation (31)..(31): N = T or C
tcatctcccaaggagagaccaccgtggcctncattgatgacgctgaggagctcatggccac

SEQ ID NO: 55 - DNA, 121 bp, Homo sapiens
  variation (61)..(61): N = A or G
cctccatgaaggaggagttcacacgcctcaaagaggcgctagagaagtccgaggctcgccncaaggagctggaggagaagatggtgtccctgctgcaggagaagaatgacctgcagctcca

SEQ ID NO: 56 - DNA, 61 bp, Homo sapiens
  variation (31)..(31): N = CT or no nucleotide
tccgtggaagaaaggtccttgaggcatcctngaggagactggagatttctcctgacagcag

SEQ ID NO: 57 - DNA, 61 bp, Homo sapiens
  variation (31)..(31): N = A or G
acaccacgttcctcctccaggacttgtgccnacagctccacgcccgtgtggacaaggtgga

SEQ ID NO: 58 - DNA, 61 bp, Homo sapiens
  variation (31)..(31): N = A or G
agatctttgaccttcgaggcaagtttaagcngcccaccctgcggagagtgaggatctctgc

SEQ ID NO: 59 - DNA, 61 bp, Homo sapiens
  variation (31)..(31): N = A or G
atgccatgatgcaggcgctgctgggggcccnggctaaggagtccctggacctgcgggccca

SEQ ID NO: 60 - DNA, 61 bp, Homo sapiens
  variation (31)..(31): N = A or G
tccaggaaaaccgggaggtgggagactggcncaagaacatcgatgcactgagtggaatgga

SEQ ID NO: 61 - DNA, 121 bp, Homo sapiens
  variation (61)..(61): N = A or G
cctgtctttgagagaaacgagctcctcctcctctttcttcctgttctcaaagtgagcctcnatcagcgcctgcaactcattcaggtccttctccatgcgcttccggtggatgtcctgtggg

SEQ ID NO: 62 - DNA, 121 bp, Homo sapiens
  variation (61)..(61): N = A or G
cacaagagaagcgctgggtcaacgtttgttgattgggcaatcaatggttgaatcttagtcnataggagagtcaggtgcacatgggaaagcctgttctggggggtttcttactgcctcagga

SEQ ID NO: 63 - DNA, 121 bp, Homo sapiens
  variation (61)..(61): N = T or C
ttcttcatcttctaaatgaaacacgagaaatcaatcaaggtccttgttctgagctcaagtncccccctccgccaccagcaagagccttccccacagataagccctagccaagatgcactct

SEQ ID NO: 64 - DNA, 61 bp, Homo sapiens
  variation (31)..(31): N = T or C
tctgcttcacttctccagagcaggaggaggnagcggaagaggatgctgaagcagaggctga

SEQ ID NO: 65 - DNA, 61 bp, Homo sapiens
  variation (31)..(31): N = T or C
gcttcacttctccagagcaggaggaggcagnggaagaggatgctgaagcagaggctgagac

SEQ ID NO: 66 - DNA, 61 bp, Homo sapiens
  variation (31)..(31): N = G or no nucleotide
ggacttcagctgtgggggccgtgtcaaccgnctgcattcctcagttctggaggtgcgatgg

SEQ ID NO: 67 - DNA, 61 bp, Homo sapiens
  variation (31)..(31): N = G or no nucleotide
ctcggcagttcgtctgtgactcagaccgggnactgcttggacggctcagacgaggcctcct

SEQ ID NO: 68 - DNA, 61 bp, Homo sapiens
  variation (31)..(31): N = A or G
caaggggacagtagcccctgctcggccttcnagttccactgcctaagtggcgagtgcatcc

SEQ ID NO: 69 - DNA, 61 bp, Homo sapiens
  variation (31)..(31): N = A or G
agcctctttctctctcttccagatatcgatnagtgtcaggatcccgacacctgcagccagc

SEQ ID NO: 70 - DNA, 61 bp, Homo sapiens
  variation (31)..(31): N = T or G
acaagtgccagtgtgaggaaggcttccagcnggacccccacacgaaggcctgcaaggctgt

SEQ ID NO: 71 - DNA, 61 bp, Homo sapiens
  variation (31)..(31): N = A or T
ccttcttcctggcttcctggtgaagatgagnggcgacctgctggagctggtgagccaccct

SEQ ID NO: 72 - DNA, 61 bp, Homo sapiens
  variation (31)..(31): N = A or G
cacaacgcttttgggggtgagggtgtctacnccattgccaggtgctgcctgctaccccagg

SEQ ID NO: 73 - DNA, 61 bp, Homo sapiens
  variation (31)..(31): N = A or C
aggggccgtgacagccgttgccatctgctgncggagccggcacctggcgcaggcctcccag

SEQ ID NO: 74 - DNA, 61 bp, Homo sapiens
  variation (31)..(31): N = A or G
ttcctggcttcctggtgaagatgagtggcgncctgctggagctggtgagccaccctttttg

SEQ ID NO: 75 - DNA, 61 bp, Homo sapiens
  variation (31)..(31): N = A or G
cttcctggcttcctggtgaagatgagtggcnacctgctggagctggtgagccacccttttt

SEQ ID NO: 76 - DNA, 61 bp, Homo sapiens
  variation (31)..(31): N = C or G
ccaggggaggacatcattggtgcctccagcnactgcagcacctgctttgtgtcacagagtg

SEQ ID NO: 77 - DNA, 61 bp, Homo sapiens; identical to SEQ ID NO: 76
  variation (31)..(31): N = T or G

SEQ ID NO: 78 - DNA, 61 bp, Homo sapiens
  variation (31)..(31): N = GCC or no nucleotide
acctctcgcagtcagagcgcactgcccgccntgcaggcccaggctgcccgccggggatacc

SEQ ID NO: 79 - DNA, 61 bp, Homo sapiens
  variation (31)..(31): N = T or C
gagaatgtgcccgaggaggacgggacccgcntccacagacaggtaagcacggccgtctgat

SEQ ID NO: 80 - DNA, 61 bp, Homo sapiens
  variation (31)..(31): N = A or C
tcacagagtgggacatcacaggctgctgccnacgtggctggtaagtcaccaccccactgcc

SEQ ID NO: 81 - DNA, 61 bp, Homo sapiens
  variation (31)..(31): N = A or G
aggccagcatggggacccgtgtccactgccnccaacagggccacgtcctcacaggtaggag

SEQ ID NO: 82 - DNA, 61 bp, Homo sapiens
  variation (31)..(31): N = A or C
ctctgtctttgcccagagcatcccgtggaanctggagcggattacccctccacggtaccgg

SEQ ID NO: 83 - DNA, 61 bp, Homo sapiens
  variation (31)..(31): N = A or T
agccggtgaccctggggactttggggaccanctttggccgctgtgtggacctctttgcccc

SEQ ID NO: 84 - DNA, 61 bp, Homo sapiens
  variation (31)..(31): N = A or G
tgatccacttctctgccaaagatgtcatcantgaggcctggttccctgaggaccagcgggt

SEQ ID NO: 85 - DNA, 61 bp, Homo sapiens
  variation (31)..(31): N = T or C
tggaatgcaaagtcaaggagcatggaatccnggcccctcaggagcaggtgaagaggcccgt

SEQ ID NO: 86 - DNA, 61 bp, Homo sapiens
  variation (31)..(31): N = C or G
cccgaggaggacgggacccgcttccacaganaggtaagcacggccgtctgatgggagggct

SEQ ID NO: 87 - DNA, 61 bp, Homo sapiens
  variation (31)..(31): N = C or G
gccagcatggggacccgtgtccactgccacnaacagggccacgtcctcacaggtaggaggc

SEQ ID NO: 88 - DNA, 61 bp, Homo sapiens
  variation (31)..(31): N = A or G
tcgagaatgtgcccgaggaggacgggacccncttccacagacaggtaagcacggccgtctg

SEQ ID NO: 89 - DNA, 61 bp, Homo sapiens
  variation (31)..(31): N = A or T
gcccgaggaggacgggacccgcttccacagncaggtaagcacggccgtctgatgggagggc

SEQ ID NO: 90 - DNA, 61 bp, Homo sapiens
  variation (31)..(31): N = A or G
ccctggggactttggggaccaactttggccnctgtgtggacctctttgccccaggggagga

SEQ ID NO: 91 - DNA, 61 bp, Homo sapiens
  variation (31)..(31): N = T or C
actgtatggtcagcacactcggggcctacanggatggccacagccgtcgcccgctgcgccc

SEQ ID NO: 92 - DNA, 61 bp, Homo sapiens
  variation (31)..(31): N = T or C
tgctccagtttctccaggagtgggaagcggnggggcgagcgcatggaggtgactgtacccc

SEQ ID NO: 93 - DNA, 61 bp, Homo sapiens
  variation (31)..(31): N = T or C
aaggaggagacccacctctcgcagtcagagngcactgcccgccgcctgcaggcccaggctg

SEQ ID NO: 94 - DNA, 61 bp, Homo sapiens
  variation (31)..(31): N = T or C
ggttggcagctgttttgcaggactgtatggncagcacactcggggcctacacggatggcca

SEQ ID NO: 95 - DNA, 61 bp, Homo sapiens
  variation (31)..(31): N = A or C
ccgggacgtcagcactacaggcagcaccagngaaggggccgtgacagccgttgccatctgc

SEQ ID NO: 96 - DNA, 61 bp, Homo sapiens
  variation (31)..(31): N = T or C
gccggggatacctcaccaagatcctgcatgncttccatggccttcttcctggcttcctggt

SEQ ID NO: 97 - DNA, 61 bp, Homo sapiens
  variation (31)..(31): N = A or G
gcaacctctcccctggccctcatgggcaccntcagctccaggcggtcctggtggccgctgc

SEQ ID NO: 98 - DNA, 61 bp, Homo sapiens
  variation (31)..(31): N = A or G
ctctgccaaagatgtcatcaatgaggcctgnttccctgaggaccagcgggtactgaccccc

SEQ ID NO: 99 - DNA, 61 bp, Homo sapiens
  variation (31)..(31): N = C or G
gcaggccttgaagttgccccatgtcgactanatcgaggaggactcctctgtctttgcccag

SEQ ID NO: 100 - DNA, 121 bp, Homo sapiens
  variation (61)..(61): N = T or G
attcttactgtgaattgttactcactcagtttgaactgaattttttttcttttttcagatnggtccaagcatacttctttgagtacacttcttccccagattcctggtgtttggcaagtct

SEQ ID NO: 101 - DNA, 121 bp, Homo sapiens
  variation (61)..(61): N = T or C
aaaagatatggggtaaactcttctgaatattttcttatacacattaaaagtacatgactcnggtttgatgccattttaatataaccttggaatttgataattaaactcaaaaccacacacg

SEQ ID NO: 102 - DNA, 121 bp, Homo sapiens
  variation (61)..(61): N = A or G
aatgttgaacacatgaacaaggctccactctgcctttggagatgaactggtccagttcttntttatagatcagaaagctaaggtcacccagcgagttaagggcagagctgggagcagagca

SEQ ID NO: 103 - DNA, 121 bp, Homo sapiens
  variation (61)..(61): N = C or G
ctgctccagatgggaggtggtggagagttgaggggtggagagtgccaaaaaggaggctgtntgggtgcccttggggatggagagatagagacccactcggagggacaggtccagccagaag

SEQ ID NO: 104 - DNA, 121 bp, Homo sapiens
  variation (61)..(61): N = T or C
acagatgagaaaactgatgcaggaagaggtagagtaacttgcttacagccacacagtcacngaggttagtattcagagccaggcggtccaatttcgaggccatctgtgaacagctctactc

SEQ ID NO: 105 - DNA, 121 bp, Homo sapiens
  variation (61)..(61): N = A or G
gcagagctagaagaaagcttttcaggatgccaagagtatttcatttgacagcacgtattcnctttcattcaagatgaacccttagctaaatctcgttattttgctatttctcatccatgaa

SEQ ID NO: 106 - DNA, 121 bp, Homo sapiens
  variation (61)..(61): N = A or G
tatattctggatatgagtccttcattagatatatgacttttatttcctttcattctggtcnttgtcttttgactttcttggtggcatcatttgcagcacaaaagttttaaattttgaagaa

SEQ ID NO: 107 - DNA, 121 bp, Homo sapiens
  variation (61)..(61): N = T or C
tccgggggcggggcctaggacggctgagcgccgctcgcgtctgctacggggcgcggagacncccccccaaggaggcggggcctaagagttcaaaggatggaactgagcgttacggaggcgt

SEQ ID NO: 108 - DNA, 121 bp, Homo sapiens
  variation (61)..(61): N = A or G
ttgattgattgattgattgattgtatttctttttagttcaacaagcatttagggagcaccnacttcatgccaagcactgtgcaaggtggtagagattcagcaataaacaaaaacaagtgta

SEQ ID NO: 109 - DNA, 121 bp, Homo sapiens
  variation (61)..(61): N = T or C
tatctgccagagaaatatagaggaacatgcctccagcaagtgcaaatataatatttggagngaaattgttgcccaccaaaatgccaaaagctagcccaacatagcaggaacatgcagaaag

SEQ ID NO: 110 - DNA, 121 bp, Homo sapiens
  variation (61)..(61): N = A or G
gtttgccagagaggaaagcaacactcatgcacctcattgcaaaacacgtctggtcaggcgnaaaaccctggacttgctgggagggagggaagaatgaagttgaaactgaaaggctgctttg

SEQ ID NO: 111 - DNA, 121 bp, Homo sapiens
  variation (61)..(61): N = C or G
cctgacagttccaaacctttaaacaaagaagggattatcagaccccattttacaataaagncatgacgccagagaagataaaagactcttatgtaatgggatgaaaatagaatccaggttt

SEQ ID NO: 112 - DNA, 121 bp, Homo sapiens
  variation (61)..(61): N = T or C
gccccttcccatttttcttttttcccctaaatttgcgcttgcttcagcattttccaaggcnaatgtttcctgcttcagctactcaattacaagctgattttttttcagggaatatttttta

SEQ ID NO: 113 - DNA, 121 bp, Homo sapiens
  variation (61)..(61): N = A or G
catgcctggggcattgccacctgagaacaggtcttacgaggggatgcgatgcctggatacnaatgactgctggattccaagctgcagcatgccccctgaacacctggccaaatgcaagagg

SEQ ID NO: 114 - DNA, 121 bp, Homo sapiens
  variation (61)..(61): N = A or C
taaaaagaagaacacagtataagtcttgattggaacccttttccttcaaaactagtttggnagactgttgagaaaagaccatggctaaaaaaagttccagagttgtaaaatccagctcttt

SEQ ID NO: 115 - DNA, 121 bp, Homo sapiens
  variation (61)..(61): N = C or G
cttcctctcagcagctctttgtttgcatggtgcagccattctcctggttatccagactcantcatcttcagcttctcaggggtccaatcctgcagtatctagtgccactgctcctttcttc

SEQ ID NO: 116 - DNA, 121 bp, Homo sapiens
  variation (61)..(61): N = A or C
ctgcatctcaggattgccctaacaactactcataatatgtgccaattcaaagtgcttggcnccaaaccttagccaattaatcctcacagccaccctgtgtgttagttaggcctcattattc

SEQ ID NO: 117 - DNA, 121 bp, Homo sapiens
  variation (61)..(61): N = T or C
aaaagaaaacagaagcatcttataagcatttccctcatgcatttaaatgaccacaattcanaggagaaagtcctcaaattcagattcaaaatcagactttttctcaagctcaacacacact

SEQ ID NO: 118 - DNA, 121 bp, Homo sapiens
  variation (61)..(61): N = A or G
ttaggtcgacggatggcatccagaatgcttattcccagtgacacctccaatgccattatcnctctcttattctttaccaagctgtgaggtggaaagatgacctctcccttttgctgaagtt

SEQ ID NO: 119 - DNA, 121 bp, Homo sapiens
  variation (61)..(61): N = A or G
caacagacttgatagaattcttttgctgttttcctctagacagctcaggctcagtttaagntccttttaaaaagcccccattttctgatctgctccttcagaaacagggcagcaaatataa

SEQ ID NO: 120 - DNA, 121 bp, Homo sapiens
  variation (61)..(61): N = A or G
gtccggaattgatgggttgtctcactgacttcaaggaaaaagccgcagatctccgtggtgngtgctaaaattcttaaaggtggtgtgtctggagtatgttccttttcatgttcagacgtgt

SEQ ID NO: 121 - DNA, 121 bp, Homo sapiens
  variation (61)..(61): N = T or C
ctggagatgcagatataccaggccttccataccaagctcacattcagccacaggacaaagngctctgaggcaaaaaggatcatttgtctgggctgtgcattcagtaatgctcgtggggagg

SEQ ID NO: 122 - DNA, 121 bp, Homo sapiens
  variation (61)..(61): N = A or G
attgtacttgtcaatagacagctggaacagcacaaaacattaacttggaaagaaaaacaanaataaaatgtatcgcctcgtttgtttagacaacaaaacctattccagcctgggcagtggt

SEQ ID NO: 123 - DNA, 121 bp, Homo sapiens
  variation (61)..(61): N = A or G
agactacatttggttttcaagcagcattaaatacaacttaaacaacgtttcaaaaaattgnggaaggttcattgtatgatttcacatgaagttgggggtacctggttactgaagtgagtta

SEQ ID NO: 124 - DNA, 121 bp, Homo sapiens
  variation (61)..(61): N = T or C
atggagggaagtaattggactcaagataaatgtgaaggtaatactaacaggccttgaaganggacagggtgtgggggaaaaggagtgatggtgaagaaggtgtggcggtgtcctttccctg

SEQ ID NO: 125 - DNA, 121 bp, Homo sapiens
  variation (61)..(61): N = A or G
attgctatacttctatgtaccctgctgcagctctccccatgtctccacctctagaggtggngttcagggatttgcataactaaaaaatttatgaaagtgttgtcctacctttctcaggaac

SEQ ID NO: 126 - DNA, 121 bp, Homo sapiens
  variation (61)..(61): N = A or G
tggagactgactcctggacaggctgtggaatgtgtggaagcaggtacagcccaggcccgcnctgaagttcggcagggaagccagaggagtccgcagcccgggagacctggcctacagcgga

SEQ ID NO: 127 - DNA, 121 bp, Homo sapiens
  variation (61)..(61): N = A or C
ctgactgtattgctccaaagctagaggttggcaatgttgaaggagcaaaggggaggtctanctttaggaagaattacatttcctggaactgggaggttggagattgtgtgtgccaaggtct

SEQ ID NO: 128 - DNA, 121 bp, Homo sapiens
  variation (61)..(61): N = T or C
agcacacgggggcactttcagtgcccgaagtgtgcatcagagaagccctgaggttccagtnccttaagaggaacccctctctcctgatccccccctccctgcctctccctctctctgtgtg

SEQ ID NO: 129 - DNA, 121 bp, Homo sapiens
  variation (61)..(61): N = T or G
aatttgggtagcattgtaaaagttatggtggggaaagaaattaatgaaaagggaaggcatncttttctttttggtgaatgaaaccagcccacatcgagaaaattattccatgtatttaata

SEQ ID NO: 130 - DNA, 121 bp, Homo sapiens
  variation (61)..(61): N = A or G
caccctctggtcttctttcttgtctcctgctcttccctccaccttgtaaggcactttttgnatggtaggactggttttggggtcctgtggtgccccatgcctgctgctgtcccagctttgg

SEQ ID NO: 131 - DNA, 121 bp, Homo sapiens
  variation (61)..(61): N = A or G
acattgaagtaagagtaagcttgttactagctctgtacaaatggaacattctacattttantaagggttttgaactcttttctcaagtgcatacaggtgccagagagagaactcacattag

SEQ ID NO: 132 - DNA, 121 bp, Homo sapiens
  variation (61)..(61): N = T or C
agttattttagaatttagggcaattagctgctctgagagatattgtgacatttgaaatgtnatcacccccaagcagatttaatgggaaccatttgggcaggttgctggcacctggatgctt

SEQ ID NO: 133 - DNA, 121 bp, Homo sapiens
  variation (61)..(61): N = T or C
tcttttgtaacccctctctcctgtgtacattccttggaacaaatcccactgggtcttggcngtgttcctcaggcacatcgtagtgtgtgacattcctcttttaaaccatcacagaactact

SEQ ID NO: 134 - DNA, 121 bp, Homo sapiens
  variation (61)..(61): N = A or C
gtcttgtctacctcctctcaaaaagataatactccatttggtctgaaaaacaatgtgagantctgaggtttgtttcctttggttctgagtgtaaggactgtgactcatggttgtccagaat

SEQ ID NO: 135 - DNA, 121 bp, Homo sapiens
  variation (61)..(61): N = T or C
gtgatgcttgaacatggttcactaagtcaaaatctgccctaaatattttaaatctctgagngtttgagaatctgggtggtctgagctcataaaacagcctctgtgaaggcacagtctctct

SEQ ID NO: 136 - DNA, 121 bp, Homo sapiens
  variation (61)..(61): N = A or G
gctaagagggctgcggcggcctgggagcagggctaccattgaacttagggtgaaagtccantcgaacccaaacctgggtggaaccggtgacttgaaatcctagggataggcggaatcaaag

SEQ ID NO: 137 - DNA, 121 bp, Homo sapiens
  variation (61)..(61): N = T or C
gtctaggagatagtgacctcaaagcttagagatgatctaaaagaatgctgcttttgctctntccaaattcaggaaatttctggtattgtctgagacttaatgtgccccaccttgggtcctt

SEQ ID NO: 138 - DNA, 121 bp, Homo sapiens
  variation (61)..(61): N = T or C
tactggatttaagtctacccccttgctgctttttgtttttggtccatctgttctttttctnttattcctctttacttgcctttcttagtatatttttctccctcctaatggtaactgagta

SEQ ID NO: 139 - DNA, 121 bp, Homo sapiens
  variation (61)..(61): N = T or G
acaatccaagaggaatcaaacatttacatcagcaagaaccacatttgtgtctgtgatactnttgaagggtccatagagagataactgatttttaaagcagtggtacttgtaaaacagcaac

SEQ ID NO: 140 - DNA, 121 bp, Homo sapiens
  variation (61)..(61): N = T or C
ccagaagaaattctcagaatttctgaaaggttacttcaaggacaaaatacctgtattcctngcctgtccagggatctgctcttacagattagaagtagtcctattagcccagaggcgatgt

SEQ ID NO: 141 - DNA, 121 bp, Homo sapiens; identical to SEQ ID NO: 140
  variation (61)..(61): N = T or C

SEQ ID NO: 142 - DNA, 121 bp, Homo sapiens
  variation (61)..(61): N = A or C
aaatccagccagctcaagaacaggaaatgaagcagaagtcagaattttatcaggtattgcnatgctctttgttgccttcagaatgatattctctggaataagttgaaaaacaatttttttc

SEQ ID NO: 143 - DNA, 121 bp, Homo sapiens
  variation (61)..(61): N = T or C
ttagtttttcttttttcaccataaatcaaatatactttgtcagaggaagattaacaatttnacttaccggttatagacctggtggtggcactttcataaagaggaactccttctccggcat

SEQ ID NO: 144 - DNA, 121 bp, Homo sapiens
  variation (61)..(61): N = T or C
gctacagctgaaggaagaacgtgagcacgaggcactgaggtgattggctgaaggcacttcngttgagcatctagacgtttccttggctcttctggcgccaaaatgtcgttcgtggcagggg

SEQ ID NO: 145 - DNA, 121 bp, Homo sapiens
  variation (61)..(61): N = A or C
gaagccaatgaacttcggagaggaagatctactgtgaccaagagtattgtggaggtaaganacagctgcagatgttaaggaaattgctgagtgatggtaacctcatattttgtctgtagat

SEQ ID NO: 146 - DNA, 121 bp, Homo sapiens
  variation (61)..(61): N = C or G
gttgtgaattttaactgctaagctctcagcctggccactgcttacatcaacagcaacctanaaagactcagcaaaatataattgaaattttaatttttgtgtcagaattgttttaccaaag

SEQ ID NO: 147 - DNA, 121 bp, Homo sapiens
  variation (61)..(61): N = A or G
ggttatgaattgacataatgagaccaaaattactattgttctgttttcttcttatttgctncctaagagccaaagaaatgtctgaggaccaggaatgtttcaaagtctctggaaaaattga

SEQ ID NO: 148 - DNA, 121 bp, Homo sapiens
  variation (61)..(61): N = A or T
ttgacttctgacaaaggtggtaaattcagacaacattatgatctaataaactttatttttnaaaaatgaccatttttccattttctttctaggaaattaaacccttttaattcttatctac

SEQ ID NO: 149 - DNA, 101 bp, Homo sapiens
  variation (51)..(51): N = A or G
ccaacaaggggctgggttagcaaaaggcttttaaagccatatacaggtaanagtcactactgccatgtgtgtgtgtttgtgtgtgtgtgtgtgtgtttgag

SEQ ID NO: 150 - DNA, 121 bp, Homo sapiens
  variation (61)..(61): N = T or G
gtaatatattcatgtagaacatgtaggaaacacaaggaagtacaacaaagacaacaaaggnagtgcctttttcatggcggtggaaacagctgcggtgtgaaattcctcctgcgtcagccag

SEQ ID NO: 151 - DNA, 121 bp, Homo sapiens
  variation (61)..(61): N = T or C
tcccattatgtgattgccatctttgccctgatgagctcctgtttagccactgcaggtaagntgcataatataaattgcatatttgttatatatttttggatatattttgtcccagtctctt

SEQ ID NO: 152 - DNA, 121 bp, Homo sapiens
  variation (61)..(61): N = T or C
aggagaccaagggtgcagttatgcctcagattcacttttatcacctttccttgcctctttnctagcactgcccaacaacaccagctcctctccccagccaaagaagaaaccactggatgga

SEQ ID NO: 153 - DNA, 121 bp, Homo sapiens
  variation (61)..(61): N = A or G
agagaactggaactagattgaaccctcagcctagcaatgtcactatgctacacttttcctngtgtggtctacccgagatgaggggctgaggtttttttttgtttttgtttctgttttgagg

SEQ ID NO: 154 - DNA, 121 bp, Homo sapiens
  variation (61)..(61): N = T or C
tgtgtggtttcccccttactggtgatggcatactccaggaattctggaaaccctccctgangccgaagcagggtcaccctctccccgcctccttgctagtccttactcctggcccagcgag

SEQ ID NO: 155 - DNA, 121 bp, Homo sapiens
  variation (61)..(61): N = T or C
tctgaattcaaagactgtattttatccatcacaaatatttgtagtactcctcctgctgttntttatgtgcaaagcactgagatacatattcttacatttattaagatttttacttacattc

SEQ ID NO: 156 - DNA, 51 bp, Homo sapiens
  variation (26)..(26): N = A or C
caatgatctcagaggctgtatacccncccagagttattttatgcatatcaa

SEQ ID NO: 157 - DNA, 121 bp, Homo sapiens
  variation (61)..(61): N = T or C
gaagaactggggccccgaccccagacacctcatggaggaaggaagagattgggaccaccgngggtggggggtgaagcggaagtcacctctggctcccacagcatcctgaccttccctgtca

SEQ ID NO: 158 - DNA, 121 bp, Homo sapiens
  variation (61)..(61): N = T or C
tctttttatgtaaggttattttgaaaatatatgctgttgagttaaaaagagcaagaacgcngccacatagctagaaaatatccacatgttgaaacctttcggtatatgtttatcagtaaag

SEQ ID NO: 159 - DNA, 121 bp, Homo sapiens
  variation (61)..(61): N = T or C
cgcctacgtctttgaacaccgtgcttccacgctctcctggcccctgtggatgggggtgccncacggctacgagatcgagttcatctttgggatccccctggacccctctcgaaactacacg

SEQ ID NO: 160 - DNA, 121 bp, Homo sapiens
  variation (61)..(61): N = A or G
tgtgtcattctctgaggaaaacaagttcttctcaaggaggaaagtctgaacttgtcaaacngtcccttaagaagccgaagttaccagaaggtcgttttgatgcaccagaggattcccattt

SEQ ID NO: 161 - DNA, 51 bp, Homo sapiens
  variation (26)..(26): N = C or T
aggccgaggcgagaggatcaggaagncaggagatccagaccatcctggcca

SEQ ID NO: 162 - DNA, 51 bp, Homo sapiens
  variation (26)..(26): N = G or T
ttatctcaaaaaattgtacatcatanggtgtgctagacatcagcaaatgca

SEQ ID NO: 163 - DNA, 51 bp, Homo sapiens
  variation (26)..(26): N = A or C
tttgggaaaagggtttagtatcgttntgctgtcattgcatcaagtcatcgt

SEQ ID NO: 164 - DNA, 51 bp, Homo sapiens
  variation (26)..(26): N = C or T
cagctcacctccagctttagttttcncatgacagtaagtctattaccctcc

SEQ ID NO: 165 - DNA, 121 bp, Homo sapiens
  variation (61)..(61): N = A or C
tttcccccggatacccagtttctcccggaggcagccaatgatctcagaggctgtatacccncccagagttattttatgcatatcaaggaaagtctacatagaggactgtttctggggtacc

SEQ ID NO: 166 - DNA, 121 bp, Homo sapiens
  variation (61)..(61): N = C or G
gttccctcaagttaacgtggaagaagtatccttttttcacattttctctgaatcttatctncatggatcttcttagaaaccttcttctagaaattatttcctcattccatatttccaacct

SEQ ID NO: 167 - DNA, 51 bp, Homo sapiens
  variation (26)..(26): N = A or C or G or T
atgtgaacatgtgggactttgtgctntcaccagatgagattaacaccatct

SEQ ID NO: 168 - DNA, 51 bp, Homo sapiens
  variation (26)..(26): N = A or C
tgcctgttcagatgactgcaaggacnagggcgactgctgcatcaactacag

SEQ ID NO: 169 - DNA, 121 bp, Homo sapiens
  variation (61)..(61): N = A or G
tagcatcttgttaaaagatcctgctgataagtggatcttatcggcaacaattagtgtggtntttgcctaatggtgattttctaattttctaatgccctccttctttttttttctttttaaa

SEQ ID NO: 170 - DNA, 121 bp, Homo sapiens
  variation (61)..(61): N = T or G
atagacagtagaatcatggttaccagaggatgggaagggtagtgtgtctggagttgggggngcttagggcaagttggggattgttaataagtacaaaaaatagaaagaatgaataagacct

SEQ ID NO: 171 - DNA, 51 bp, Homo sapiens
  variation (26)..(26): N = G or T
acctgaaaataggtgagctgtcaagntgttggcagggagaggctcctctgg

SEQ ID NO: 172 - DNA, 51 bp, Homo sapiens
  variation (26)..(26): N = A or T
ggttccttgcgactgctgtgaatttngtgatgcacttggatagtctctgtt

SEQ ID NO: 173 - DNA, 121 bp, Homo sapiens
  variation (61)..(61): N = T or C
agaaggatcatctgatgtcaccccctctaattttgagtgatccagtttctttgctttttangcttgtatctattcttccatcgtagactgacctggtcatttctttggagtgttttttggt

SEQ ID NO: 174 - DNA, 51 bp, Homo sapiens
  variation (26)..(26): N = A or C
atctctgtctcttaattatctcacanagccaggtattttttattgttagct

SEQ ID NO: 175 - DNA, 121 bp, Homo sapiens
  variation (61)..(61): N = T or C
tgaaagagaagaccaaccacatccaggccctggcacacagcaccgtgggtcagaccttgcnggtgagagtccagccgtgacaaagggacccaggtggcagttgcagccaggccttcctgga

SEQ ID NO: 176 - DNA, 51 bp, Homo sapiens
  variation (26)..(26): N = C or T
ggggcagcctcatgggcctggccaangctgctgcctcctatgaaatgctca

SEQ ID NO: 177 - DNA, 51 bp, Homo sapiens
  variation (26)..(26): N = A or G
tccgtaccatcaagtcatttcctctngacgtctgaacctgcactcagggtc

SEQ ID NO: 178 - DNA, 51 bp, Homo sapiens
  variation (26)..(26): N = C or G
gaagtaaactcgaatgttgattatangttttctatcaaattattcaagtat

SEQ ID NO: 179 - DNA, 121 bp, Homo sapiens
  variation (61)..(61): N = A or T
ctgaccaggaacatttaccccacagagtcagcagagaggcagctgttctggttgttttttnaatctgtacaaataaacaaagacttaaagtgatcaattatatttgtacccattaaatact

SEQ ID NO: 180 - DNA, 121 bp, Homo sapiens
  variation (61)..(61): N = A or G
ctgtacaaagtgggtgctacagaagcattagctattgcatggtggtggctgtgcgattgtnctattggatagagtcaaggggtctggttctcaaccttgatctgcattaagcagctatgga

SEQ ID NO: 181 - DNA, 51 bp, Homo sapiens
  variation (26)..(26): N = C or T
aacatccaagttccgcatatgaacanggctctcctactgtatttcttgtta

SEQ ID NO: 182 - DNA, 51 bp, Homo sapiens
  variation (26)..(26): N = A or C or G
ctgaaccagcaaagagaaaagaaggnccccagaaatcacaggtgggcacgt

SEQ ID NO: 183 - DNA, 51 bp, Homo sapiens
  variation (26)..(26): N = G or T
agtaaggtaggatggacagtagattnaagatactgattgtgtttgcaaaca

SEQ ID NO: 184 - DNA, 51 bp, Homo sapiens
  variation (26)..(26): N = A or C
tcattagataagatccatacgagttnatcctgcctatcaagaaaaggactt

SEQ ID NO: 185 - DNA, 51 bp, Homo sapiens
  variation (26)..(26): N = C or G
taacagatgctggtggggttgtggangaaagggaatgcttatacactgtta

SEQ ID NO: 186 - DNA, 51 bp, Homo sapiens
  variation (26)..(26): N = A or C or G or T
gggcagactgggccctgcacctcccngggctgctagcatttgcaggcctac

SEQ ID NO: 187 - DNA, 51 bp, Homo sapiens
  variation (26)..(26): N = A or G
gcagttacattctgaaagccagtagntggagacccaggagaactgatggtg

SEQ ID NO: 188 - DNA, 51 bp, Homo sapiens
  variation (26)..(26): N = A or C or G
tgatgccaacagcccactctacgacntggcacccagcgacctgcaccacca

SEQ ID NO: 189 - DNA, 51 bp, Homo sapiens
  variation (26)..(26): N = C or T
cgctggcgggcacggtacctgggctnggcagggtcctctgccaggcgtgtc

SEQ ID NO: 190 - DNA, 121 bp, Homo sapiens
  variation (61)..(61): N = A or C
cgtgtggggtaagcagatgacagggcagtgacagggtgatcgggcccggtcagtggtcccnggggtcggggacaggtgggaatcgaagtcataggaggcggggcaaaggccacggggaaag

SEQ ID NO: 191 - DNA, 121 bp, Homo sapiens
  variation (61)..(61): N = A or G
agccgagaaatgttgattattaggctgagtagtttgaactcaacaaaggcaatgtaaaatnacatttcagggactgcttagtgcaacaaaacacttacatggggccattagaccctaacta

SEQ ID NO: 192 - DNA, 121 bp, Homo sapiens
  variation (61)..(61): N = A or G
atgtcaaaaagttttgtggatgttgatgctgccactgatgttggaaacaacaccactttgnacacagggaggacctcacaggtagaagtgttcagtaagagaagagagaaatccgctaact

SEQ ID NO: 193 - DNA, 121 bp, Homo sapiens
  variation (61)..(61): N = A or T
actgcgtctaggctgctgtgctcctcagagacaagaggctgtgcagaaaggtggtgggtgngggggcagaggccaggtcatgggcaagggggcttgaagcatgtttttaatgcagtctttc

SEQ ID NO: 194 - DNA, 121 bp, Homo sapiens
  variation (61)..(61): N = T or C
agggttcagtatcaggaattagagaactcttcttgctgacctgcctataaaggatttgatngtggaagcctgtccgtattactgattcagtagctcctctgaatctatttactcacaacct

SEQ ID NO: 195 - DNA, 121 bp, Homo sapiens
  variation (61)..(61): N = T or C
ttaatgaaaacttacacaaatttgtggaaaacaacttagaaaggaagaaagctctaagatnaataacctaagcttccaccttgggaaactagaaaaaggataatttgttagtcactaaagt

SEQ ID NO: 196 - DNA, 121 bp, Homo sapiens
  variation (61)..(61): N = C or G
tatttgtatgctcaaaatagttatatattcattaatgaattcaatatcctgttcatactanttagagctggtcagcctttttgggtaacacagttaatttaccaactgatacagataatag

SEQ ID NO: 197 - DNA, 51 bp, Homo sapiens
  variation (26)..(26): N = A or G
aaattgtcattggatttattattacngaagttattgctatcttaaggagag

SEQ ID NO: 198 - DNA, 51 bp, Homo sapiens
  variation (26)..(26): N = C or T
tgcttctttatcaacagcagccagcngggacagccaagtggttcggagaga

SEQ ID NO: 199 - DNA, 121 bp, Homo sapiens
  variation (61)..(61): N = C or G
gagtgagtgggccacaggcagagcctattattagctccccacgaggcctgggctctttggnaacacgtaggacgtggacagttttactactgcgacattgggctctgtctccaggcacccc

SEQ ID NO: 200 - DNA, 51 bp, Homo sapiens
  variation (26)..(26): N = C or T
ctgtcctgcttgtcatttcttgtctngctgattaactctatggatggggca

SEQ ID NO: 201 - DNA, 51 bp, Homo sapiens
  variation (26)..(26): N = C or G
gaaagttctcaacatttataactacnagcagtatgtaagagagttatggtt

SEQ ID NO: 202 - DNA, 51 bp, Homo sapiens
  variation (26)..(26): N = G or T
tgcccaggaatatccaggcaagaatnaccatattctgataattactcaggc

SEQ ID NO: 203 - DNA, 51 bp, Homo sapiens
  variation (26)..(26): N = A or T
tctcggatatggcgaccgaagtgatntggggcccttgtcaagggtctctat

SEQ ID NO: 204 - DNA, 51 bp, Homo sapiens
  variation (26)..(26): N = G or T
cattaaagttttgctggggttggggntgagggtgtagagagcaaaagtgtg

SEQ ID NO: 205 - DNA, 51 bp, Homo sapiens
  variation (26)..(26): N = C or T
tagagagctaagcactttttagatantatataatttaattgccgtatgagg

SEQ ID NO: 206 - DNA, 121 bp, Homo sapiens
  variation (61)..(61): N = T or C
tcaaaacctagcacagctgttatttactgaacaattagagagctaagcactttttagatantatataatttaattgccgtatgaggcacccttagttttcagacgagaaaccacagttaca

SEQ ID NO: 207 - DNA, 121 bp, Homo sapiens
  variation (61)..(61): N = T or G
atctggactagtgacttgaaaatttaaatgtatattgtagagggatttcgaggtgaaaatnctaatcggggtttgactttcaaagcgtgtaagctacagatcgttttgataagattccact

SEQ ID NO: 208 - DNA, 121 bp, Homo sapiens
  variation (61)..(61): N = T or G
ggcttgcattcctcagattaaaagtttcttttttttgtttgttttccaccaggctgagatnctgtttctaacaaacctcccgatgctgctgatgctgcatatctgaagaccacactttgag

SEQ ID NO: 209 - DNA, 121 bp, Homo sapiens
  variation (61)..(61): N = A or G
ccccaccccctggaaagacagtggtcacagaatagaaagaacacaggaactggggttgacnagcatgaatatgggccacagccccacccctgaggggcttgacacaggctttagagccaca

SEQ ID NO: 210 - DNA, 121 bp, Homo sapiens
  variation (61)..(61): N = A or C
atggggaaagcaatgttggaatagagggcaagcacatatggccagggtcggacttctcagnccacatttgctgagggaacagatgaagcaacaaagttgggaagtaacagactgggccatt

SEQ ID NO: 211 - DNA, 51 bp, Homo sapiens
  variation (26)..(26): N = A or G
gagccccaggaactggagcgaaagtnagatttgccccatgaggaaaagctg

SEQ ID NO: 212 - DNA, 51 bp, Homo sapiens
  variation (26)..(26): N = A or G
cactattttcttgacccctacttacnatcctgggagatgtatttgggttta

SEQ ID NO: 213 - DNA, 51 bp, Homo sapiens
  variation (26)..(26): N = C or G
aactctgggagattctcctattgacncagaaagcgattccttcactgatac

SEQ ID NO: 214 - DNA, 61 bp, Homo sapiens
  variation (31)..(31): N = T or A
cccagccttgccttcatgatgcaggcccaantgcacccttgcagacaacagtctggcctga

SEQ ID NO: 215 - DNA, 61 bp, Homo sapiens
  variation (31)..(31): N = T or C
ccagaaaaaaaatgattttctttcacgaagntccaaacaggtttctctcctgttccccagc

SEQ ID NO: 216 - DNA, 61 bp, Homo sapiens
  variation (31)..(31): N = G or A
tgggacagtgtggctcgtgtccttcccaacngctccctcttccttccggctgtcgggatcc

SEQ ID NO: 217 - DNA, 121 bp, Homo sapiens
  variation (61)..(61): N = A or T
aagtagtttcagggggtctccctacatttagcaacatgagagaatttactactttttatanttttaaacatgagtgtgctatcactgttattatctagcagatagtgctagatacgtgcca

SEQ ID NO: 218 - DNA, 121 bp, Homo sapiens
  variation (61)..(61): N = T or C
cattggcatcagctaattttaaagaggtaaaacattgaattaattcatctttagtggaggnagcaattagtatatcatccatataatgaagaatgtaattatttttaaaagtctgttggat

SEQ ID NO: 219 - DNA, 121 bp, Homo sapiens
  variation (61)..(61): N = A or G
ggtcctagagctggatattgtagcaagaagagaaagctcattgagaggcgagggcggagcntgggcagaggtgagggtgtgcttggtgtatcctagcctggcaggtatatggagccactgt

SEQ ID NO: 220 - DNA, 121 bp, Homo sapiens
  variation (61)..(61): N = C or G
cagatgtcccctatgtgtaatgcagagcaattactagatattagctgtgtgctagttactntttatagcattattaaaagtgcttgtcagccatgctacaagctgattaccaaaagaaaat

SEQ ID NO: 221 - DNA, 121 bp, Homo sapiens
  variation (61)..(61): N = C or G
tatgctggcaaagctgcccctcctccaggcccccagtgatgctaagaattcacaccatctnctatccagaaccagtaactgcctgggaggttcctgatgggaatattctgcctatgcaggt

SEQ ID NO: 222 - DNA, 121 bp, Homo sapiens
  variation (61)..(61): N = C or G
gtgcagagctaaatgaatgtggaggtgaagtgcggggaagtatcgcagataaacttccaantgtctggcttagcactgggtggcttgtggggcatgtttggtggggacaagataggctgtg

SEQ ID NO: 223 - DNA, 121 bp, Homo sapiens
  variation (61)..(61): N = T or C
aaataagtgaagtagataaatgcagttaatactaactgccagaaggaaaatgaagcagggnggcatgacaatgcttagggtggtgggggtattataattcctctggccgtaacttcatttc

SEQ ID NO: 224 - DNA, 121 bp, Homo sapiens
  variation (61)..(61): N = T or C
gaccatatattagattgaacatctttccaccatacttctcttccttggtttactgtagganggttaccagagaaatgtttttctggcattagtataagtgagttgatgaaattgttctgca

SEQ ID NO: 225 - DNA, 121 bp, Homo sapiens
  variation (61)..(61): N = T or C
ggtctgacagaacctctgacagagcagaagggctgttcctgggctgtcggcctctgtctanaatcctcaggaagccttctgaataaaactaactttaattatttaaaagttttgccaggcg

SEQ ID NO: 226 - DNA, 121 bp, Homo sapiens
  variation (61)..(61): N = T or C
tccttactctctctagagctcacaactttgtgttgcttttacagataagaaaattgagttncagggcagtaagttaatttgctagcattccacagacagagttgggtttggaaccaggtct

SEQ ID NO: 227 - DNA, 121 bp, Homo sapiens
  variation (61)..(61): N = T or C
ctattcttattttaaatcagtgatcagaacataatctgtcatatcatatatttttcatacncagatatttattcaaccattttcctcaaagacccaataaaatagagagccacagcctatc

SEQ ID NO: 228 - DNA, 121 bp, Homo sapiens
  variation (61)..(61): N = C or G
atgctgtgggccctgattgtaaataaaaatgcttttgcggaaacagcaaaatttggggaancctgtcttccacctccttgactgaaaagtctggtcggaggtgccacctggctacttcctc

SEQ ID NO: 229 - DNA, 121 bp, Homo sapiens
  variation (61)..(61): N = T or C
cggcatttcctgctccagccaggtgtggagtgggggattctcctgccagggcttactgtgntcctgagagggcctgtggttgtcatggtgaccaggagtggcctcagcagggctgaccatc

SEQ ID NO: 230 - DNA, 121 bp, Homo sapiens
  variation (61)..(61): N = T or C
tagcataagaatgaggtactggtggactggtttgaggtgtggtgtgaggccatttgacagngtccacaaaaaaagctaggttggtcctgctctgtgagcagcatgctgtacattcaggggc

SEQ ID NO: 231 - DNA, 121 bp, Homo sapiens
  variation (61)..(61): N = A or G
tgaaacccacatacaaagctttaccatttacacaggcatagctttagtgtgtacatcagcntctcctggggttggagttgttggcctgcgatggagcctgggaacctgcattttataaaaa

SEQ ID NO: 232 - DNA, 121 bp, Homo sapiens
  variation (61)..(61): N = A or G
acagaaacaccatcatttctttgtaatcagctgaaatcaccactcattagtgggcctctcntgggctataactgattttacagccacctgtcatttcttttttctttacaactttgtgcgt

SEQ ID NO: 233 - DNA, 121 bp, Homo sapiens
  variation (61)..(61): N = T or G
attgcatcactcagggaccctgctggagggagagtctgttatcttttgagcatacagtagntattcaaaaagtacaacacttggtgaatgagaagattggcattagtgatggaactgctca

SEQ ID NO: 234 - DNA, 121 bp, Homo sapiens
  variation (61)..(61): N = T or C
tagtaatctatgaccagatgtactcttacactgaagccttgatttgattctgctataatanaacttctgagccagtttgatgtgattttgcatgtactgaacctccaccacctgtatctaa

SEQ ID NO: 235 - DNA, 121 bp, Homo sapiens
  variation (61)..(61): N = A or G
aatggtttgctcacttttgtgattttgtgatgtgtcagtgctgggactgaatccaagttgngtgacagctggggcgatgcagcagaaggcaggtcttgctttttggtaacacagtgagctt

SEQ ID NO: 236 - DNA, 121 bp, Homo sapiens
  variation (61)..(61): N = A or C
gctggatcctctttctggccttgcaatgtgaggaatctaggctgggtgctgtgcttagtcncctagccacctgggagcttgcttaaaaatgcaaatcttctgggctctgacaacaaggttt

SEQ ID NO: 237 - DNA, 121 bp, Homo sapiens
  variation (61)..(61): N = A or G
ctgggagcttgcttaaaaatgcaaatcttctgggctctgacaacaaggttttgtggggcangtctaggaatctgattccaatacttgaggtccttgacctaactgttgggaatgaagatgt

SEQ ID NO: 238 - DNA, 121 bp, Homo sapiens
  variation (61)..(61): N = T or C
tatattttttggtatctggcttcttttgttcaatacaacatatttgtgattaatccatgcngttgtgtaggtcaatcatctttattgctgagtagtattccattgcaaggtcataccataa

SEQ ID NO: 239 - DNA, 121 bp, Homo sapiens
  variation (61)..(61): N = T or C
ttaatgtctccctaagggttccttaccatagcttcctgctaaggacaggggccaaggtagnccctcacacagaggtcccaggttctgccacatggccctgccctccctggacacagctgat

SEQ ID NO: 240 - DNA, 121 bp, Homo sapiens
  variation (61)..(61): N = T or G
aacaaccttgtgcttgcaattggtatctgaagttgggggccatctcgtggggctgagcccnttagtggtggggtctgtgctaactctcagaattaaattgtaagacacccagttaatgcct

SEQ ID NO: 241 - DNA, 121 bp, Homo sapiens
  variation (61)..(61): N = A or G
aggagaggagaggctggcggctcccaggagcagatcacctgcccccactgcaaggcccccntgtgctcaataaactgacccttgcgcagagactaaggctcacttattggaaactattatt

SEQ ID NO: 242 - DNA, 121 bp, Homo sapiens
  variation (61)..(61): N = A or G
agaatctggaggaaagagaactgggatgagagtcaggagacagatgtttaagacttgtctntgcctttgactgaatgatttgggctaatcacttggattttctgtccttcaggaggatcaa

SEQ ID NO: 243 - DNA, 121 bp, Homo sapiens
  misc_feature (61)..(61): n is a, c, g, or t
cattttatctataagtattaaatatcttagcatgtatctctgaaagatcaagactttttanatacataaccgccataccattggtcacacctaaaaaattaacaatacttttctttttctt

SEQ ID NO: 244 - DNA, 121 bp, Homo sapiens
  variation (61)..(61): N = A or G
tttcagccagacattttaatgtagttgtggacatctactggttctaccccattgcttcccntccttcctcagtcctctgggcgccattctctggaaatgttcctctgtgctgtgtgtgaca

SEQ ID NO: 245 - DNA, 121 bp, Homo sapiens
  variation (61)..(61): N = A or T
gcgttgggccaagctcctgtgtgtggcccattgggctgtgctgctaagcctctccacaagncttgcccagagctattgagcttccgagtggcttcatttggcctaacattggatgctttga

SEQ ID NO: 246 - DNA, 52 bp, Homo sapiens
  variation (27)..(27): N = T or G
tggggagcccactggggtcagaggctnccccatccttgacgccgaggcccct

SEQ ID NO: 247 - DNA, 121 bp, Homo sapiens
  variation (61)..(61): N = A or G
acggactgaactttggtgtaagagagaggtggcacattgcattcaggaaaggagacttcgntaattagagctagaagaattggttttctgtatgaaaaaggaaaaaacccgtatgttatgc

SEQ ID NO: 248 - DNA, 121 bp, Homo sapiens
  variation (61)..(61): N = T or C
tcaccaggttaactccagagcttgggagaaacaaagtcccctccctccacctgagttgctnggactcctagtggaaaagtgagtcacagagggaggctctctgtccctctcacataatggg

SEQ ID NO: 249 - DNA, 121 bp, Homo sapiens
  variation (61)..(61): N = A or G
tgttgtcaacagtgagatatatcctcattagtaacaaatgttgaagataaagcctttcatngatagctggccggaggattgggatacttatgagggggaaatataaggctaaatatacttt

SEQ ID NO: 250 - DNA, 121 bp, Homo sapiens
  variation (61)..(61): N = C or G
tatcagtgatactcaaaatgttaaacaaccagttcagcgcgaggggcaactgatcagaaangatatggtcacatcgattacctggaaatatttcttgtagggcagatgtaaaagatagttc

SEQ ID NO: 251 - DNA, 121 bp, Homo sapiens
  variation (61)..(61): N = A or C
ccctgggtgggtctgaggctgcgttctcttggaggtttttgagcccaggtatggctttgcncctgtgatttatgtctttaagtgtttgtgaatcacttctctacgtgcgtgtgtgtttgtt

SEQ ID NO: 252 - DNA, 121 bp, Homo sapiens
  variation (61)..(61): N = T or C
catatcgctgaagagatccgtttcttccttatctcactggtctctctgctataccaacacntttggctcagcctcagctccagccagcctcagttccggaggagccccctcaaattcatgg

SEQ ID NO: 253 - DNA, 121 bp, Homo sapiens
  variation (61)..(61): N = T or C
aatagtaggcctaagttacacgcattcacacttcctttaaacagtacacctttctgtattncagagagaggcctgggtccccaagtatctataggcgtatgttgtcttttcttgggcccag

SEQ ID NO: 254 - DNA, 121 bp, Homo sapiens
  variation (61)..(61): N = T or C
acctcttattctctacatgcaaaaaattaaatattttgtgttcaaataaaattagaaaacntgagtggcctcttgtgtcctgcagagatttaaaacatggcatctcaatatttttgagaac

SEQ ID NO: 255 - DNA, 121 bp, Homo sapiens
  variation (61)..(61): N = A or C
ttaaaattggaaacacttcttgctaccagggatggggggtggggctcagcgtttggggaancggagtgggagtcttttgctgaacagacttttgttttcttaagttttgaactacatgaat

SEQ ID NO: 256 - DNA, 121 bp, Homo sapiens
  variation (61)..(61): N = T or C
attgggcacctatgggttgcctagagacaagttaccatggtcctcttccctcgcccgtcangtgcagggtcaaaggcaggtgagctacacagctcgccctgctcttcactctgctttaatg

SEQ ID NO: 257 - DNA, 121 bp, Homo sapiens
  variation (61)..(61): N = A or G
aataattccacatggctcatctaaaattagagagaagtgaccaggatttttagttgactgnctttatagtagatgcaagatagtcaaaatctatttaaatgactttcttagggaattggag

SEQ ID NO: 258 - DNA, 121 bp, Homo sapiens
  variation (61)..(61): N = A or G
gtgggcagcaaggaaacccgaagccatcctggaagacctgtgtcttccagggaaaaagggnctcagggctcagcccaggcagggaaggctgaggggagtggggctgtcctatgcagatgac

SEQ ID NO: 259 - DNA, 121 bp, Homo sapiens
  variation (61)..(61): N = T or C
ttgagctatgtaaacttaaaattattattttttctttttcaagagatagggtcctgcttangttgcccaggccacagtacagtggctattcatagacgtgataataacacactcccgggct

SEQ ID NO: 260 - DNA, 121 bp, Homo sapiens
  variation (61)..(61): N = T or G
tattccaggttctgagacaaaccaaaagctgctataatagttgagaaagcaactgtgtccnaagatgagctaaaataaaaaatccattcattgtcataaaaagtcacatctcattaaagat

SEQ ID NO: 261 - DNA, 121 bp, Homo sapiens
  variation (61)..(61): N = A or G
gttcagttattaggtctgtaatcttttgtatgatctgtgtccacataaaaaattaccaacnttttcccaactagtttagtagtgaaagtaccatctaatttaaaaaaaaatcttcagtgtg

SEQ ID NO: 262 - DNA, 121 bp, Homo sapiens
  variation (61)..(61): N = A or G
ggatgaatggatggataagaaggggtgggctgaggtagcctggaatagcagcatcttcaantgctctaaacaggacaagtgaagagataggggatgtcaacaatcttccatcagggcaata

SEQ ID NO: 263 - DNA, 121 bp, Homo sapiens
  variation (61)..(61): N = A or G
acagggccaccagagcagggatctcaggctctttaattccttccctcctggggtctacacncattgctacggccccatcccagagccaggccgggtgcaaagccctggagacagatgccgc

SEQ ID NO: 264 - DNA, 121 bp, Homo sapiens
  variation (61)..(61): N = A or G
caagaataaatatttgtgactttaggtaatattttcttggacattactttaaaagtgcaantgacaaaagaaaaatatagataaattggatctcatcaatattaaaaactttgtgctttaa

SEQ ID NO: 265 - DNA, 121 bp, Homo sapiens
  variation (61)..(61): N = C or G
tggtgtctgcacacatcctgcttaggattttcccgcccgatacctgtaccccgggttttgngctgacacatgctccattgcttcctcgtgagagctttgcctttatctcaggcggggtgcg

SEQ ID NO: 266 - DNA, 121 bp, Homo sapiens
  variation (61)..(61): N = A or G
cacccacgtgatgatgccgggttgtaaatcactaaagaaggcccctccttcccttgaccantagctccccaaaatgtatgcgatgaggtggtaaagtgccatggttgtgccactatgccaa

SEQ ID NO: 267 - DNA, 121 bp, Homo sapiens
  variation (61)..(61): N = A or G
gctgggctacctcctttgggagataggttctgccctgtcactgtctacaaaattgttaatnttcccaaagaaactgtctgggcccccaagccctcttttaagccaggaattgtgacatttt

SEQ ID NO: 268 - DNA, 121 bp, Homo sapiens
  variation (61)..(61): N = A or G
ttgtatggttcaaggtgagttgtaataggagaattaagagagctgaaatgataggagatgntggtaagagatttttgggtatgaggttgatataaccaaggaacctgatgttttaaaggaa

SEQ ID NO: 269 - DNA, 121 bp, Homo sapiens
  variation (61)..(61): N = T or C
aacaaagctctgtggatacacacacaggcatgtacatgcacaaactctctctctctcatanagtttcagcaggtcataaatgagaacccagctacaatgaaatgatacagcttttactgat

SEQ ID NO: 270 - DNA, 121 bp, Homo sapiens
  variation (61)..(61): N = A or C
ttaaaattactttctaaagccaatcattctgcctaatacagggtcttcatttatttttagntacctgaaactgagtctaaaaccacttctctctacttcctcttgtctttttcatttaaac

SEQ ID NO: 271 - DNA, 121 bp, Homo sapiens
  variation (61)..(61): N = A or T
gacttcagttaacatttattctgcttccagacccccagagcccctggttctgctgcctctnagcagagtgcccttagttaattaacctctctgagcctcagttttctcttctgtaaaatgg

SEQ ID NO: 272 - DNA, 121 bp, Homo sapiens
  variation (61)..(61): N = A or C
ctctgaaggccctctgagctctaacagggccagcatgaacccgagaacaaggtctccttcnatctgtgccctacacacctcacttgctttaccacagtccctgagctctaaaatgctctga

SEQ ID NO: 273 - DNA, 121 bp, Homo sapiens
  variation (61)..(61): N = T or C
gcatctcacttcttactaagacaaggctattccatcctcttgacaacaagataggctttanttcgcctctagaatggcctccagccctatctctaggtcaccagctctttcgcaaatgaca

SEQ ID NO: 274 - DNA, 121 bp, Homo sapiens
  variation (61)..(61): N = T or C
gaaatacaaacaccatttccttttattaattggctgtgtggccttgagcaacttaatctcnctgtgcctcgggtttcctatgtgtaagatagccgggcctatttcatataaggttattatg

SEQ ID NO: 275 - DNA, 121 bp, Homo sapiens
  variation (61)..(61): N = T or C
aggacgtgatgctgggtgattttagaggagaaaccatgaaggattccaaggagcagcagtnggtcaggtgggaagaaaggagtgtgacatcacagaagatgaaaaataaaagtattttaag

SEQ ID NO: 276 - DNA, 121 bp, Homo sapiens
  variation (61)..(61): N = A or G
ttgcattggttgattttcagctattgaaccatcattgtatacttggaaaaaatcctacctngtcatggcgcatacttatttttacatattactaaactgtatttgctaatattttgtttaa

SEQ ID NO: 277 - DNA, 51 bp, Homo sapiens
  variation (26)..(26): N = A or G
cagtgtggctcgtgtccttcccaacngctccctcttccttccggctgtcgg

SEQ ID NO: 278 - DNA, 121 bp, Homo sapiens
  variation (61)..(61): N = A or C
aagcagggcaatcaaggcatcccgggggtggctatgaagagtgaatgagatagcagacaanccagatgcctaccgacaggtgaagggaccaacacagtgcggtataggcgtataagggaat

SEQ ID NO: 279 - DNA, 121 bp, Homo sapiens
  variation (61)..(61): N = A or G
ccatggggcgggagcagcagagtctgatgttgtgtacttcagggagctggagttctatganggaagagcgaggaggcatgtgggaggaagaacagccccactgaggcctgcagggaaggca

SEQ ID NO: 280 - DNA, 121 bp, Homo sapiens
  variation (61)..(61): N = T or C
aaacccaaccccctctgagtattaaactatagtggcattgtccctcaagctcccctctgcnttggctccagagtcttcctcctcttcttccagactgggcagggtggctgttgttattggt

SEQ ID NO: 281 - DNA, 121 bp, Homo sapiens
  variation (61)..(61): N = A or G
accttaacactgagttccctccaaaaatcagtgctcacatctctcctaacagaggactagnatgacctactcttccctgaccttttctctgtctcataaaaatgtaatagtctcgtcattt

SEQ ID NO: 282 - DNA, 121 bp, Homo sapiens
  variation (61)..(61): N = T or C
ttcagctgcaagaggcttatcctggcttttacattgcagcatggtttttaattttattttntacatttttttaaaatcaggcagcctccagaatcacagaagattcagagaaactcccaac

SEQ ID NO: 283 - DNA, 121 bp, Homo sapiens
  variation (61)..(61): N = A or G
aagtgattgctgccaagtacctctgccacacaggttataccacccttgaacaattcttccncagcaaatgcagtgagaacagcagccaatgctcagaaaccaggttatacgagtaacttgt

SEQ ID NO: 284 - DNA, 121 bp, Homo sapiens
  variation (61)..(61): N = T or C
taaagcttagagagagacagaagtctgacacgtcttgggaatctccttcataataaattanaggtgttcaagaaataccagtgatttaattaaaatgtaaatgaagttgcttcggacccat

SEQ ID NO: 285 - DNA, 51 bp, Homo sapiens
  variation (26)..(26): N = 289BP ALU or no nucleotide
acctgctgcctatacagtcacttttnttttatgtggtttcgccaattttat

SEQ ID NO: 286 - DNA, 51 bp, Homo sapiens
  variation (26)..(26): N = A or C or G or T
taaattcacagtcaaagaatcaagcncttttcgaaacattgaagttgtttt

SEQ ID NO: 287 - DNA, 61 bp, Homo sapiens
  variation (31)..(31): N = C or G
caccaccttcactgtctccaagtactggtgntaccgtctgttgtccacgctgctgggcgtc

SEQ ID NO: 288 - DNA, 121 bp, Homo sapiens
  variation (61)..(61): N = T or C
ggaaggtgagctacaccaccttcactgtctccaagtactggtgctaccgtctgttgtccangctgctgggcgtcccactggccctgctctggggcttcctgttcgcctgcatctccttctg

SEQ ID NO: 289 - DNA, 121 bp, Homo sapiens
  variation (61)..(61): N = A or G
aggagacagttcagcagggtggcaacatgtcgggcctggcccgcaggtccccccgcagcgngtgacggcaagctggaggccctctacgtcctcatggtactgggattcttcggcttcttca

SEQ ID NO: 290 - DNA, 121 bp, Homo sapiens
  variation (61)..(61): N = C or G
cctattttattatttaaattgcagcaggagggaagcatgtctactttatccaatttcacanagacgctggaagacgtcttccgaaggatttttattacttatatggacaattggcgccaga

SEQ ID NO: 291 - DNA, 61 bp, Homo sapiens
  variation (31)..(31): N = T or C
acggagttagagggtggcttcagtcggcaangcaagcgcaagttgtccttccgcaggcgca

SEQ ID NO: 292 - DNA, 61 bp, Homo sapiens
  variation (31)..(31): N = T or C
gagggcccaggccgcagctccagccccctcngcctggtgcccttctccagccccaggcccc

SEQ ID NO: 293 - DNA, 61 bp, Homo sapiens
  variation (31)..(31): N = A or G
gagcggcggctggaccacttctctgtcgacngctatgacagttctggtgagaacccctcag

SEQ ID NO: 294 - DNA, 61 bp, Homo sapiens
  variation (31)..(31): N = A or G
accaccctgtacatcggcttcctgggcctcntcttctcctcgtactttgtgtacctggctg

SEQ ID NO: 295 - DNA, 121 bp, Homo sapiens
  variation (61)..(61): N = C or G
acaatggggtgactcctggagagaagatgctcacagtcccccatatcacgtgcgacccccnagaagagcggcggctggaccacttctctgtcgacggctatgacagttctggtgagaaccc

SEQ ID NO: 296 - DNA, 121 bp, Homo sapiens
  variation (61)..(61): N = T or C
aagatgatgaatgtctcgaaccagctgtgctccacgatgtggtagcaggtcttgcgcaacngccaccagaccttccctggggcctgtgtggtgtccaccgcacagcagggacagcgccgga

SEQ ID NO: 297 - DNA, 121 bp, Homo sapiens
  variation (61)..(61): N = A or G
tggccagccaggccgaggctcacacgatggactcacggtccctgtccggagaaggggggangtcggcgagatcttcactgtggctgtagtcagacccccgcacctggaggttatcgctggt

SEQ ID NO: 298 - DNA, 121 bp, Homo sapiens
  variation (61)..(61): N = A or C
tggaggagatggaggagctggagggtgggccaaggggtcgggagaagttctcactcatcangtaggcgatgaggccctctcgctcaggggcatcctcttcggagaggccgctgcccgcctg

SEQ ID NO: 299 - DNA, 121 bp, Homo sapiens
  variation (61)..(61): N = C or G
gataacctccaggtgcgggggtctgactacagccacagtgaagatctcgccgacttccccncttctccggacagggaccgtgagtccatcgtgtgagcctcggcctggctggccaggacac

SEQ ID NO: 300 - DNA, 61 bp, Homo sapiens
  variation (31)..(31): N = GCCCTC or no nucleotide
cccagtcccggaacctcggctcctggccacncatggcaaaaagaacagcactgtggactgc

SEQ ID NO: 301 - DNA, 61 bp, Homo sapiens
  variation (31)..(31): N = A or G
tggctgtcccctcagagttagaggagtctcnccacaagtgtccaccatgctggaaccgtct

SEQ ID NO: 302 - DNA, 61 bp, Homo sapiens
  variation (31)..(31): N = T or C
ccgagatgggccccatcaagtcactgcggangctgcgtgcactccgtcctctgagagctct

SEQ ID NO: 303 - DNA, 61 bp, Homo sapiens
  variation (31)..(31): N = T or C
ttctctttgcacttagggggccaggacatcntcatgacagaggagcagaagaagtactaca

SEQ ID NO: 304 - DNA, 121 bp, Homo sapiens
  variation (61)..(61): N = A or G
tcacctgaaatgactgatatagttttcagggcccggaggactcggaaggtgcgtaaggctnagacattgcccaggtccacaaattcagttgtgtatctgtaacaagggaaattcacacacg

SEQ ID NO: 305 - DNA, 51 bp, Homo sapiens
  variation (26)..(26): N = A or G
ggagactgaggcaggagaattgcttnaaccctggaggcagaggttgcagtg

SEQ ID NO: 306 - DNA, 51 bp, Homo sapiens
  variation (26)..(26): N = C or T
gtacaagctgcaccccaggtgagccngccccgctctctccctggtaaagtg

SEQ ID NO: 307 - DNA, 51 bp, Homo sapiens
  variation (26)..(26): N = A or C or G
ggcccgagacggggaggagggggagngcccaaggggaggaggcggggtccg

SEQ ID NO: 308 - DNA, 51 bp, Homo sapiens
  variation (26)..(26): N = G or T
gcccggggcggggcacagggggcggnggagtgggcggggcccgaggccggg

SEQ ID NO: 309 - DNA, 51 bp, Homo sapiens
  variation (26)..(26): N = C or G
caagggcaggaggctgggacaggacngggatgcaaagggaggggcggggcc

SEQ ID NO: 310 - DNA, 51 bp, Homo sapiens
  variation (26)..(26): N = A or C
tccccatccgtgagtcccctcggtgntccctgcccgccgtggccatcctct

SEQ ID NO: 311 - DNA, 51 bp, Homo sapiens
  variation (26)..(26): N = A or G
cgggggtggcggggtcgcggagcctnagcaggactgcgccgtgacctccgg

SEQ ID NO: 312 - DNA, 121 bp, Homo sapiens
  variation (61)..(61): N = A or T
aactaatcctcattactcaagtgagaacagaggtagtaggcttgacattgacatgtttttnctattaataattttaattggctgaggtcaattctatcactataagcattttatatttatg

SEQ ID NO: 313 - DNA, 121 bp, Homo sapiens
  variation (61)..(61): N = A or G
attaaaattattgagttaaatattcattcaatgtgtggctatatgtgagtgtgcttgcacncgcatgtgtgtgtgaatgttgaaatcagaagtatgacggtctcttgatccactgaaatga

SEQ ID NO: 314 - DNA, 121 bp, Homo sapiens
  variation (61)..(61): N = T or C
tgcatgtttgtttactttttaattgaagtataacatacatacaagaaaatacacaaatcanaaatatataactctatgagttttcacaaagtaagcacctctgtgcattcaataccaatat

SEQ ID NO: 315 - DNA, 121 bp, Homo sapiens
  variation (61)..(61): N = A or C
catctcagtacaggacatcttcatccttccagttgttcagaccaaaaaccctggaaacatncttgacttcgtctttcccattccacatttcacccatcagcaaattctgttgcatctgtct

SEQ ID NO: 316 - DNA, 121 bp, Homo sapiens
  variation (61)..(61): N = A or G
aaaaaaaaaagaaagtcatgataaagagtgtggattttattttacgcaaagtgggagctcnttaagaaggtaaaagtttttaagcaagggaatgacatgatttgacctactttttaaagga

SEQ ID NO: 317 - DNA, 121 bp, Homo sapiens
  variation (61)..(61): N = A or G
cattgacttccttatttcttatatggccccacctttcctagtcacgtgtcttggtactatntgagatttcagatccacctgcctacctgtaactcttgattccttttcatttccattgctg

SEQ ID NO: 318 - DNA, 51 bp, Homo sapiens
  variation (26)..(26): N = C or T
tagcagttcaggtcctaaggcatganattgattaagtgtctgatgagaatt

SEQ ID NO: 319 - DNA, 121 bp, Homo sapiens
  variation (61)..(61): N = A or T
agggccatttatctatgagacactacaggcattgtgtctagccctgtgggtttacattagntagggtaggttattgctgcaacgtaccctaacttgatatgatttttgctgcaaaaatcat

SEQ ID NO: 320 - DNA, 51 bp, Homo sapiens
  variation (26)..(26): N =G or T
gtgtttcagcttggcacacagaaacngttttaatttaacagtccagctcct

SEQ ID NO: 321 - DNA, 121 bp, Homo sapiens
  variation (61)..(61): N = T or C
gaactgggtattatttgttgatggacatgtaccatagcatttttctggagcgtaatttcanaatgtgaatcagaagtcttaatagtgcatacgttttgacctagtgaatctcctgctaaga

SEQ ID NO: 322 - DNA, 121 bp, Homo sapiens
  variation (61)..(61): N = A or G
atatttagtactggcatttcttcttcactgatttttcattctacctgtctttagtatcatnggggtagttacctcagcgggggtagggagaccttgggtgtctcttacttgtacatactct

SEQ ID NO: 323 - DNA, 121 bp, Homo sapiens
  variation (61)..(61): N = A or T
cctttaatagatcaattctctattgtggtttgaatttggtgcactcccaatttactctaancttctacgggcttccttggagaaactggggcagagatgccgaagactcatttcgggtaca

SEQ ID NO: 324 - DNA, 51 bp, Homo sapiens
  variation (26)..(26): N = A or C
catgccagttgcccactgtggcaatnaatatctgagcctgtggtttttgcc

SEQ ID NO: 325 - DNA, 121 bp, Homo sapiens
  variation (61)..(61): N = A or C
cctgtgacagtgccagcttcatagcctagtctaggcatgccagttgcccactgtggcaatnaatatctgagcctgtggtttttgccttaggtaaactgtagagatggactcatggaatgct

SEQ ID NO: 326 - DNA, 121 bp, Homo sapiens
  variation (61)..(61): N = A or G
ttttaatgctgaatgatattcatcactgtctgaaatgggcttatttatgtacgaatgtttnttcctatctcttcctccattctcccaaaatgtaaaccccataagaacaggaatcttgtct

SEQ ID NO: 327 - DNA, 121 bp, Homo sapiens
  variation (61)..(61): N = T or C
tttatgaattttttttttaaacaggggaaggtacctcttttcaaggtgtcattattaagcncatgggactcttcaattttaatgttatatcaaagagagtaaatttcttgtgattattgtg

SEQ ID NO: 328 - DNA, 121 bp, Homo sapiens
  variation (61)..(61): N = T or C
ttatgttaactactgttagctgctttagcctgttgttaactgcaaactccttttacattanctagcacaggcctgggcacaagtatgatatttaagtagatgcttagtgactgattgatgt

SEQ ID NO: 329 - DNA, 121 bp, Homo sapiens
  variation (61)..(61): N = C or G
ttttcagaatattataaaccacccttccccaaagttttatttccgcaatcactccctaatntttatttcttttttgcttcgcatcatatacatttttctcttcaggacaccttcttttccc

SEQ ID NO: 330 - DNA, 121 bp, Homo sapiens
  variation (61)..(61): N = A or G
aaaatgtaaaccccataagaacaggaatcttgtctgtcttagtcacactcagtatccttanagccttgtgtgtagtaggtgtgtagtgaattttggttgaatgagtggcagttctaataat

SEQ ID NO: 331 - DNA, 51 bp, Homo sapiens; identical to SEQ ID NO: 172
  variation (26)..(26): N = A or G

SEQ ID NO: 332 - DNA, 121 bp, Homo sapiens
  variation (61)..(61): N = A or T
agttatgcatttagaatgtctgaattattattctaggttccttgcgactgctgtgaatttngtgatgcacttggatagtctctgttactctaaagttttaataggtaacagtcagaaatgg

SEQ ID NO: 333 - DNA, 121 bp, Homo sapiens
  variation (61)..(61): N = A or G
tgtaatgaagttttaggcctcagcttccctgaactggagtgtttttccttcaccttttccngtctctgggttgcatcgccagactgtctctaagcccaacaaacgcgttccttccaggcaa

SEQ ID NO: 334 - DNA, 121 bp, Homo sapiens
  variation (61)..(61): N = T or C
aagttggaaagagcagacttgctgagtcttccagtcttcaactttctcccgtgctggatgntctctgctcttgaacatcaaactccaagttcttcagcttttggactcttggacttatgct

SEQ ID NO: 335 - DNA, 51 bp, Homo sapiens
  variation (26)..(26): N = A or C
gaaatgtcaatcagagcccggagccncgggaatctccgccaatctgttcgg

SEQ ID NO: 336 - DNA, 121 bp, Homo sapiens
  variation (61)..(61): N = A or G
gatggaaaacattggtcgctaatagcagaaacttgagaataattggtaattttcaatcctntagaaaactagactaggtctgatgactttgacctttgttacgctcttcttccaaattttc

SEQ ID NO: 337 - DNA, 121 bp, Homo sapiens
  variation (61)..(61): N = A or C
ttttataagttcaaaatgatcaagttaaaagtctgaaatttgagttgcagctttaagtganctatgacccgatgtttgtgtaaaatttatccataagtctcgtctatccaatttcttctta

SEQ ID NO: 338 - DNA, 121 bp, Homo sapiens
  variation (61)..(61): N = T or C
ccacttccctcgcacggtccgctggttccatcggctgaggagcattggtaagggtgaatgnggaggtgggccccagcccacctggggtatggggtacagggcggtgggctggggtaggaca

SEQ ID NO: 339 - DNA, 121 bp, Homo sapiens
  variation (61)..(61): N = T or C
agtgaggcaggagcctccagcagagaggcctctgcaaggttttgccttcatattataaaanacagacctttaggggacataaaaagaaatcctacaccctcccacgcttaagctaatgagg

SEQ ID NO: 340 - DNA, 121 bp, Homo sapiens
  variation (61)..(61): N = A or G
gttatcctgatttggttaatataaaaagctctatagaaatcatacctcaagcctagggganaaaaggaattgcattaatgagccttgagcccaacaatggcctcaagataactcatcccac

SEQ ID NO: 341 - DNA, 121 bp, Homo sapiens
  variation (61)..(61): N = A or G
gcattttccttccaacatacttctagtttgtttttatctggagcatggaaaataagcaacngtgaaagtcaggactagaaaatgaccttttatattgccataaatgcaacccccagaacta

SEQ ID NO: 342 - DNA, 121 bp, Homo sapiens
  variation (61)..(61): N = A or G
gtcgtcagctggaagtacagtgtggggaacattggcttactttcaatctttaatttcaaantgcggtaactaggcatttaaaaacattgtacgtaagacaaagggccactgtatggcagct

SEQ ID NO: 343 - DNA, 51 bp, Homo sapiens
  variation (26)..(26): N = G or T
ccttcacctgtcggtaggcatctggnttgtctgctatctcattcactcttc

SEQ ID NO: 344 - DNA, 51 bp, Homo sapiens
  variation (26)..(26): N = C or G
tcaccatctgatgtactgttttcctnatctgtttattgtcatttttcccca

SEQ ID NO: 345 - DNA, 51 bp, Homo sapiens
  variation (26)..(26): N = C or T
actatagtacaatgtctttaccaaantggaagaccatagtgcagtcttcga

SEQ ID NO: 346 - DNA, 121 bp, Homo sapiens
  variation (61)..(61): N = C or G
cagattatttcggttctaagcaacagatactgatactgactcttaccaaacaaagcatgancaaacaaagatttatcagaagggtgcttgttagtacctgtattcaaagggagaactagtc

SEQ ID NO: 347 - DNA, 121 bp, Homo sapiens
  variation (61)..(61): N = T or G
agccctgaaagtaggccagcagactaaggcagagataatagaaggaatcatagtgtcatcngaggctgataattcatagtagagtattagtgtttgggagtgaaaagtctctatgtgaagt

SEQ ID NO: 348 - DNA, 121 bp, Homo sapiens
  variation (61)..(61): N = A or C
ggaagcggaaactttccatggttttgtggttacttagttacgaagccaataccaacctatnaattgattaaatgtaattctatagcttttgtccatcacacacaaatgatgattacaatgt

SEQ ID NO: 349 - DNA, 121 bp, Homo sapiens
  variation (61)..(61): N = T or C
ttgttatggtccttaggaaaatgttattttccttatttggaaaatggatataatcaaaatnatggaagaaagtttgtacagggtaaatctatagacgttgcctgccatcctggctacgctc

SEQ ID NO: 350 - DNA, 121 bp, Homo sapiens
  variation (61)..(61): N = T or C
tttcagtggtctagatagactctggatatattttgtgccctctactcccagaactaagagntttagaatacagtccctgaatgaaagttgtaaaagttagggcactcaagtttgtgtggac

SEQ ID NO: 351 - DNA, 121 bp, Homo sapiens
  variation (61)..(61): N = A or G
tgtaaagccagtataatattctggtcaatttgccatagttgtgctggcttctaatatgccntcaacatatataagcaaagtctcatttttcaattggaggtcccaaaactccttagcatgt

SEQ ID NO: 352 - DNA, 121 bp, Homo sapiens
  variation (61)..(61): N = A or G
ttggaaacattcactccaaaataatttcttgatttttccctgatacttactgcagggccangttccccaggaggaccaggatctccaggaaaaccaacaggaccctagaatgacgttttga

SEQ ID NO: 353 - DNA, 121 bp, Homo sapiens
  variation (61)..(61): N = T or C
ttttaagctcctcagcttattcttattctcagagagggttgaaggttgacaatcccacagngttgtctggcactaattgatttgacctactttttgagtattcagacaaagaaggaggaat

SEQ ID NO: 354 - DNA, 121 bp, Homo sapiens
  variation (61)..(61): N = T or C
aacccagcccagggaaggcggagaggaagcagtgagctgttttacaatccatcaaattaantgccccaaatgggaaatttgcaggcctcctacagcctcctgtgtattccataatctccca

SEQ ID NO: 355 - DNA, 61 bp, Homo sapiens
  variation (31)..(31): N = A or G
actttgctggcagacctgagccacttacctntaagggccgggggcattttttctttcttaa

SEQ ID NO: 356 - DNA, 61 bp, Homo sapiens
  variation (31)..(31): N = A or T
aggaattctctctcctcacctgcagcatcanctgttacctcaccttcggagacaagatcaa

SEQ ID NO: 357 - DNA, 61 bp, Homo sapiens
  variation (31)..(31): N = T or C
ccgatcattccccagattcagcagcgactgnaggaggagctagaccacgaactgggccctg

SEQ ID NO: 358 - DNA, 61 bp, Homo sapiens
  variation (31)..(31): N = A or T
actgaacactgaaggagaaatccagatcganggtgtgtcttgggattcaataactttgcaa

SEQ ID NO: 359 - DNA, 61 bp, Homo sapiens
  variation (31)..(31): N = T or G
acattggaatgcagatgagaatagctatgtntagtttgatttataagaaggtaatacttcc

SEQ ID NO: 360 - DNA, 61 bp, Homo sapiens
  variation (31)..(31): N = CTT or no nucleotide
tatgcctggcaccattaaagaaaatatcatntggtgtttcctatgatgaatatagatacag

SEQ ID NO: 361 - DNA, 61 bp, Homo sapiens
  variation (31)..(31): N = C or G
taactgcaggttgggctcagatctgtgatanaacagtttcctgggaagcttgactttgtcc

SEQ ID NO: 362 - DNA, 61 bp, Homo sapiens
  variation (31)..(31): N = A or G
ttggagaaggtggaatcacactgagtggagntcaacgagcaagaatttctttagcaaggtg

SEQ ID NO: 363 - DNA, 61 bp, Homo sapiens
  variation (31)..(31): N = A or C
tctctaattttctatttttggtaataggacntctccaagtttgcagagaaagacaatatag

SEQ ID NO: 364 - DNA, 61 bp, Homo sapiens
  variation (31)..(31): N = A or T
tggcactcctcatggggctaatctgggagtngttacaggcgtctgccttctgtggacttgg

SEQ ID NO: 365 - DNA, 61 bp, Homo sapiens
  variation (31)..(31): N = A or T
gagagctggcttcaaagaaaaatcctaaacncattaatgcccttcggcgatgttttttctg

SEQ ID NO: 366 - DNA, 61 bp, Homo sapiens
  variation (31)..(31): N = C or G
atttattttttctggaacatttagaaaaaanttggatccctatgaacagtggagtgatcaa

SEQ ID NO: 367 - DNA, 61 bp, Homo sapiens
  variation (31)..(31): N = A or T
agcacagtggaagaatttcattctgttctcngttttcctggattatgcctggcaccattaa

SEQ ID NO: 368 - DNA, 61 bp, Homo sapiens
  variation (31)..(31): N = A or G
cttcctatgacccggataacaaggaggaacnctctatcgcgatttatctaggcataggctt

SEQ ID NO: 369 - DNA, 61 bp, Homo sapiens
  variation (31)..(31): N = T or C
gaaggtggaatcacactgagtggaggtcaangagcaagaatttctttagcaaggtgaataa

SEQ ID NO: 370 - DNA, 61 bp, Homo sapiens
  variation (31)..(31): N = A or G
ttggaatgcagatgagaatagctatgtttantttgatttataagaaggtaatacttccttg

SEQ ID NO: 371 - DNA, 61 bp, Homo sapiens
  variation (31)..(31): N = A or G
tagttcttggagaaggtggaatcacactgantggaggtcaacgagcaagaatttctttagc

SEQ ID NO: 372 - DNA, 61 bp, Homo sapiens
  variation (31)..(31): N = T or G
gtttcctatgatgaatatagatacagaagcntcatcaaagcatgccaactagaagaggtaa

SEQ ID NO: 373 - DNA, 61 bp, Homo sapiens
  variation (31)..(31): N = T or C
tgtgtggtattttctttcttttctagaacanaccaaataattagaagaactctaaaacaag

SEQ ID NO: 374 - DNA, 61 bp, Homo sapiens
  variation (31)..(31): N = A or G
tctttggtgtttcctatgatgaatatagatncagaagcgtcatcaaagcatgccaactaga

SEQ ID NO: 375 - DNA, 61 bp, Homo sapiens
  variation (31)..(31): N = AGTGGT or no nucleotide
ggaagccttgcggcctgctggagtcggggtntgaagcaacgtaagtctgcatctgccttta

SEQ ID NO: 376 - DNA, 61 bp, Homo sapiens
  variation (31)..(31): N = T or G
actcctttaaacaacctgtgttgccttattntgtagacacatgtgtatggtaatgcgaggt

SEQ ID NO: 377 - DNA, 52 bp, Homo sapiens
  variation (27)..(27): N = A or G
gttccagaaaaggtaaatgctttaatngtcactgtagtacatcaatattatg

SEQ ID NO: 378 - DNA, 52 bp, Homo sapiens
  variation (27)..(27): N = C or T
gctgagtgccaattacaattgcacaantttccaatggatgaacactcctgcc

SEQ ID NO: 379 - DNA, 52 bp, Homo sapiens
  variation (27)..(27): N = A or G
aagacagttgtatgtccaatcatacancagaaattgggaaagatcttgacta

SEQ ID NO: 380 - DNA, 52 bp, Homo sapiens
  variation (27)..(27): N = A or G
acctgacagcttccgcttctttaccanggaatcccttgctgctattgaacaa

SEQ ID NO: 381 - DNA, 52 bp, Homo sapiens
  variation (27)..(27): N = A or G
ctgatgactttgggttgtgtttctcantatctttactatgactgctgttggt

SEQ ID NO: 382 - DNA, 52 bp, Homo sapiens
  variation (27)..(27): N = T or C
cctgaagccgcgccctctgcagatcantggggtggatcccgaagggtccatc

SEQ ID NO: 383 - DNA, 61 bp, Homo sapiens
  variation (31)..(31): N = C or no nucleotide
actcttctcagacgtgtatcaggagatgccngcccagctccgcaagcagcaggagtctctg

SEQ ID NO: 384 - DNA, 61 bp, Homo sapiens
  variation (31)..(31): N = T or C
gcccgccacctgcagacctacggggagcacnacccactggatcacttcgataagtgagacc

SEQ ID NO: 385 - DNA, 52 bp, Homo sapiens
  variation (27)..(27): N = T or C
gcaccagtgacgacagttcagcgtacngctcggtggatgaggtcaattactg

SEQ ID NO: 386 - DNA, 61 bp, Homo sapiens
  variation (31)..(31): N = T or C
acgaggctcccttgacaggcggctttgcatnggaaatcagctctacagttcaggtagagta

SEQ ID NO: 387 - DNA, 61 bp, Homo sapiens
  variation (31)..(31): N = T or no nucleotide
gcaggaggaatgtttcttgaacctagaggcncctatatcaagagtatgtggttatgacaca

SEQ ID NO: 388 - DNA, 61 bp, Homo sapiens
  variation (31)..(31): N = A or T
tgtgtgaacaaggaattgttggatttggaancggaattgcggtcactggagctactgccat

SEQ ID NO: 389 - DNA, 61 bp, Homo sapiens
  variation (31)..(31): N = A or G
atctttttaactgtggaagcctcactatccngtccccttggggctgtgttggtcatggggc

SEQ ID NO: 390 - DNA, 61 bp, Homo sapiens
  variation (31)..(31): N = T or C
tttttctctttatttcaggttcatgtgatcngagaggtagcttccatggcaaaagaaaagc

SEQ ID NO: 391 - DNA, 61 bp, Homo sapiens
  variation (31)..(31): N = A or G
ttttcttttctctttcagtctgtgatcaaancagggcgactgctaatcagtcacgaggctc

SEQ ID NO: 392 - DNA, 61 bp, Homo sapiens
  variation (31)..(31): N = T or C
tttctctttcagtctgtgatcaaaacagggngactgctaatcagtcacgaggctcccttga

SEQ ID NO: 393 - DNA, 61 bp, Homo sapiens
  variation (31)..(31): N = T or G
ttctgcattccccacggaggaggtggtcctngcatggggcccatcggagtgtaagttctgg

SEQ ID NO: 394 - DNA, 61 bp, Homo sapiens
  variation (31)..(31): N = T or no nucleotide
agcccctctattacgtggcagagagttttanatgatgccaaggagaaagtaaggtgaggtg

SEQ ID NO: 395 - DNA, 61 bp, Homo sapiens
  variation (31)..(31): N = A or G
tgtctcctcaccctccccattctctcttctnggagaatgatgtaaacctgacccacattga

SEQ ID NO: 396 - DNA, 61 bp, Homo sapiens
  variation (31)..(31): N = A or G
gcaggaaactctctgactttggacaggtganccacggcagcctgagctgctcagttagggg

SEQ ID NO: 397 - DNA, 61 bp, Homo sapiens
  variation (31)..(31): N = T or no nucleotide
tctcagattgactttccattccagatttacnggtttactgtggagtttgggctctgcaaac

SEQ ID NO: 398 - DNA, 52 bp, Homo sapiens
  variation (27)..(27): N = T or C
tgtattctagtgggcagcccatccctngagtggaatacatggaggaagaaaa

SEQ ID NO: 399 - DNA, 121 bp, Homo sapiens
  variation (61)..(61): N = A or G
agtcttccactgcacacagtacatcagacatggatccaagcccatgtataccccccaaccntgagtactgtcctccagctaccagttgccaggcacaatgagcgccatcttttcctgctgc

SEQ ID NO: 400 - DNA, 52 bp, Homo sapiens
  variation (27)..(27): N = T or C
agacaccatggtgcatctgactcctgnggagaagtctgccgttactgccctg

SEQ ID NO: 401 - DNA, 52 bp, Homo sapiens
  variation (27)..(27): N = C or G
ctattggtctattttcccacccttagnctgctggtggtctacccttggaccc

SEQ ID NO: 402 - DNA, 52 bp, Homo sapiens
  variation (27)..(27): N = C or G
ttggtggtgaggccctgggcaggttgntatcaaggttacaagacaggtttaa

SEQ ID NO: 403 - DNA, 52 bp, Homo sapiens
  variation (27)..(27): N = C or G
cttagacctcaccctgtggagccacancctagggttggccaatctactccca

SEQ ID NO: 404 - DNA, 52 bp, Homo sapiens
  variation (27)..(27): N = C or G
gaagttggtggtgaggccctgggcagnttggtatcaaggttacaagacaggt

SEQ ID NO: 405 - DNA, 121 bp, Homo sapiens
  variation (61)..(61): N = A or C
agcagggagggcaggagccagggctgggcataaaagtcagggcagagccatctattgcttncatttgcttctgacacaactgtgttcactagcaacctcaaacagacaccatggtgcatct

SEQ ID NO: 406 - DNA, 121 bp, Homo sapiens
  variation (61)..(61): N = CT or no nucleotide
gcttctgacacaactgtgttcactagcaacctcaaacagacaccatggtgcatctgactcngaggagaagtctgccgttactgccctgtggggcaaggtgaacgtggatgaagttggtggt

SEQ ID NO: 407 - DNA, 121 bp, Homo sapiens
  variation (61)..(61): N = ATCT or no nucleotide
ccttaggctgctggtggtctacccttggacccagaggttctttgagtcctttggggatctngtccactcctgatgctgttatgggcaaccctaaggtgaaggctcatggcaagaaagtgct

SEQ ID NO: 408 - DNA, 121 bp, Homo sapiens
  variation (61)..(61): N = C or no nucleot
aacagacaccatggtgcatctgactcctgaggagaagtctgccgttactgccctgtggggnaaggtgaacgtggatgaagttggtggtgaggccctgggcaggttggtatcaaggttacaa

SEQ ID NO: 409 - DNA, 121 bp, Homo sapiens
  variation (61)..(61): N = G or no nucleotide
aactgtgttcactagcaacctcaaacagacaccatggtgcatctgactcctgaggagaagntctgccgttactgccctgtggggcaaggtgaacgtggatgaagttggtggtgaggccctg

SEQ ID NO: 410 - DNA, 121 bp, Homo sapiens
  variation (61)..(61): N = T or G
aaggctggattattctgagtccaagctaggcccttttgctaatcatgttcatacctcttancttcctcccacagctcctgggcaacgtgctggtctgtgtgctggcccatcactttggcaa

SEQ ID NO: 411 - DNA, 121 bp, Homo sapiens
  variation (61)..(61): N = A or G
ctcaaacagacaccatggtgcatctgactcctgaggagaagtctgccgttactgccctgtngggcaaggtgaacgtggatgaagttggtggtgaggccctgggcaggttggtatcaaggtt

SEQ ID NO: 412 - DNA, 61 bp, Homo sapiens
  variation (31)..(31): N = A or G
aatggtcagaccaggaagtttctgctggacngggatgaagtcatcataacaggtgagggct

SEQ ID NO: 413 - DNA, 61 bp, Homo sapiens
  variation (31)..(31): N = A or G
cgcacaaggtggccaaggtccaggagctccngaataaagggaagaaagtcgccatggtggg

SEQ ID NO: 414 - DNA, 61 bp, Homo sapiens
  variation (31)..(31): N = T or C
cccatgctctttgtgttcattgccctgggcnggtggctggaacacttggcaaaggtaacag

SEQ ID NO: 415 - DNA, 61 bp, Homo sapiens
  variation (31)..(31): N = A or G
ccatgctctttgtgttcattgccctgggccngtggctggaacacttggcaaaggtaacagc

SEQ ID NO: 416 - DNA, 52 bp, Homo sapiens
  variation (27)..(27): N = C or G
tgctctttgtgttcattgccctgggcnggtggctggaacacttggcaaaggt

SEQ ID NO: 417 - DNA, 52 bp, Homo sapiens
  variation (27)..(27): N = T or C
tcccttgatgaggatgccgttctgcgnggccaccccggtgcccaccatgaca

SEQ ID NO: 418 - DNA, 52 bp, Homo sapiens
  variation (27)..(27): N = T or G
gctctttgtgttcattgccctgggccngtggctggaacacttggcaaaggta

SEQ ID NO: 419 - DNA, 61 bp, Homo sapiens
  variation (31)..(31): N = T or C
cggacttccaggcagtgccaggctgtggaantgggtgcaaagtcagcaacgtggaaggcat

SEQ ID NO: 420 - DNA, 121 bp, Homo sapiens
  variation (61)..(61): N = T or G
agggccacgcccaagtccacgtacctctttacagtatttggtgactgccacgcccaagggntgttcactgctggcctccgcagtccccaccacagccagaaccttcctgaggggcagtgtg

SEQ ID NO: 421 - DNA, 61 bp, Homo sapiens
  variation (31)..(31): N = C or G
cgtcaccaagaacattcacgagtcctgcatnagccagatgtaaggcttgccgttgccctcc

SEQ ID NO: 422 - DNA, 52 bp, Homo sapiens
  variation (27)..(27): N = T or C
ccctgttccgtgaggaccagatctacngcatcgaccactacctgggcaagga

SEQ ID NO: 423 - DNA, 52 bp, Homo sapiens
  variation (27)..(27): N = A or G
acgtggggaaccccgatggagagggcnaggccaccaaagggtacctggacga

SEQ ID NO: 424 - DNA, 52 bp, Homo sapiens
  variation (27)..(27): N = T or C
tgcccgaaaacaccttcatcgtgggcnatgcccgttcccgcctcacagtggc

SEQ ID NO: 425 - DNA, 121 bp, Homo sapiens
  variation (61)..(61): N = A or G
accatctccttgcccaggtagtggtcgatgcggtagatctggtcctcacggaacagggagnagatgtggttggacagccggtcagagctctgcaggtccctcccgaagggcttctccacga

SEQ ID NO: 426 - DNA, 51 bp, Homo sapiens
  variation (26)..(26): N = A or G
cacggtgccccgcgggtccaccaccnccacttttgcagccgtcgtcctcta

SEQ ID NO: 427 - DNA, 121 bp, Homo sapiens
  variation (61)..(61): N = C or G
gaggtcacaggggcagtggtgggacacacttaccagatggtggggtagatcttcttcttgnccaggtcaccctgtggcagagggaacaggtgtgtggttagaagtggctggggacacgacc

SEQ ID NO: 428 - DNA, 51 bp, Homo sapiens
  variation (26)..(26): N = A or C
agcaaccaagcttatcttacccaaantaaggtagtatatttctgttagagt

SEQ ID NO: 429 - DNA, 51 bp, Homo sapiens
  variation (26)..(26): N = C or G
aatgggcaacgggtctgaacagacanctcaccaagaagacatacagatacc

SEQ ID NO: 430 - DNA, 51 bp, Homo sapiens
  variation (26)..(26): N = T or no nucleotide
agagagactcctgtcatttatagtgntgctaacacaacttcgtagccactg

SEQ ID NO: 431 - DNA, 51 bp, Homo sapiens
  variation (26)..(26): N = A or G
cctgccccatgcagtgacctgtgacnttaaattcagaaactacctcattcc

SEQ ID NO: 432 - DNA, 51 bp, Homo sapiens
  variation (26)..(26): N = A or G
attgaacgtgtcattggcagaaaccngagcccctgcatgcaggacaggggc

SEQ ID NO: 433 - DNA, 51 bp, Homo sapiens
  variation (26)..(26): N = C or G
tggcccgcatggagctgtttttattnctgaccttcattttacagaacttta

SEQ ID NO: 434 - DNA, 51 bp, Homo sapiens
  variation (26)..(26): N = A or C or T
aaatttgtgtcttctgttctcaaagnatctctgatgtaagagataatgcgc

SEQ ID NO: 435 - DNA, 51 bp, Homo sapiens
  variation (26)..(26): N = A or C or G or T
atctgctccattattttccagaaacntttcgattataaagatcagcaattt

SEQ ID NO: 436 - DNA, 51 bp, Homo sapiens
  variation (26)..(26): N = C or T
aataagagaactggaaataacctcantaggaaatttagaacaaatacgatg

SEQ ID NO: 437 - DNA, 51 bp, Homo sapiens
  variation (26)..(26): N = C or T
tcccaacccagagatgtttgaccctngtcactttctggatgaaggtggmaa

SEQ ID NO: 438 - DNA, 51 bp, Homo sapiens
  variation (26)..(26): N = A or C
tctctcatgtttgcttctccyttcantctggagacagagctctgggagagg

SEQ ID NO: 439 - DNA, 51 bp, Homo sapiens
  variation (26)..(26): N = A or C or G
atggggaagaggagcattgaggacyntgttcaagaggaagcccrctgcctt

SEQ ID NO: 440 - DNA, 51 bp, Homo sapiens
  variation (26)..(26): N = C or G or T
gatttgtgtgggagagggcctggccngcatggagctgtttttattcctgac

SEQ ID NO: 441 - DNA, 51 bp, Homo sapiens
  variation (26)..(26): N = A or G or T
gtcttaacaagaggagaaggcttcantggatccttttgtggtccttgtgct

SEQ ID NO: 442 - DNA, 51 bp, Homo sapiens
  variation (26)..(26): N = A or G
tcacttctgtgctacatgacaacaangaatttcccaacccagagatgtttg

SEQ ID NO: 443 - DNA, 51 bp, Homo sapiens
  variation (26)..(26): N = C or T
aatcgttttcagcaatggaaagaganggaaggagatccggcgtttctccct

SEQ ID NO: 444 - DNA, 51 bp, Homo sapiens
  variation (26)..(26): N = A or C or G
ttcccactatcattgattatttcccnggaacccataacaaattacttaaaa

SEQ ID NO: 445 - DNA, 51 bp, Homo sapiens
  variation (26)..(26): N = C or T
atggtactgctcttctttggaatggngtttcatcatctgtacatcaaaaga

SEQ ID NO: 446 - DNA, 51 bp, Homo sapiens
  variation (26)..(26): N = A or G
acatcaggattgtaagcaccccctgnatccaggtaaggccaagttttttgc

SEQ ID NO: 447 - DNA, 51 bp, Homo sapiens
  variation (26)..(26): N = C or T
cctatgtttgttattttcaggaaaanggatttgtgtgggagagggcctggc

SEQ ID NO: 448 - DNA, 51 bp, Homo sapiens
  variation (26)..(26): N = A or G
gaggaccgtgttcaagaggaagcccnctgccttgtggaggagttgagaaaa

SEQ ID NO: 449 - DNA, 51 bp, Homo sapiens
  variation (26)..(26): N = C or T
tttcccactatcattgattatttccngggaacccataacaaattacttaaa

SEQ ID NO: 450 - DNA, 51 bp, Homo sapiens
  variation (26)..(26): N = A or G
cggcgtttctccctcatgackctgcngaattttgggatggggaagaggagc

SEQ ID NO: 451 - DNA, 51 bp, Homo sapiens
  variation (26)..(26): N = A or C or T
tgcttcctgatcaaaatggagaaggnaaaatgttaacaaaagcttagttat

SEQ ID NO: 452 - DNA, 51 bp, Homo sapiens
  variation (26)..(26): N = A or C
tgtggtgcacgaggtccagagatacnttgaccttctccccaccagcctgcc

SEQ ID NO: 453 - DNA, 51 bp, Homo sapiens
  variation (26)..(26): N = A or T
ttgacaccactccagttgtcaatggntttgcctctgtgccgcccttctacc

SEQ ID NO: 454 - DNA, 51 bp, Homo sapiens
  variation (26)..(26): N = A or G
gtcttaacaagaagagaaggcttcantggattctcttgtggtccttgtgct

SEQ ID NO: 455 - DNA, 51 bp, Homo sapiens
  variation (26)..(26): N = A or G or T
aatggaaagaaatggaaggagatccngcgtttctccctcatgacgctgcgg

SEQ ID NO: 456 - DNA, 51 bp, Homo sapiens
  variation (26)..(26): N = C or T
cagagctctgggagaggaaaactccntcctggccccactcctctcccagtg

SEQ ID NO: 457 - DNA, 51 bp, Homo sapiens
  variation (26)..(26): N = C or T
gatggggaagaggagcattgaggacngtgttcaagaggaagcccgctgcct

SEQ ID NO: 458 - DNA, 51 bp, Homo sapiens
  variation (26)..(26): N = A or C
tcattcctaggaaaagcacaaccaancatctgaatttactattgaaagctt

SEQ ID NO: 459 - DNA, 51 bp, Homo sapiens
  variation (26)..(26): N = C or T
ccggcgtttctccctcatgacgctgnggaattttgggatggggaagaggag

SEQ ID NO: 460 - DNA, 51 bp, Homo sapiens
  variation (26)..(26): N = A or C or T
gagatccggcgtttctccctcatgangctgcggaattttgggatggggaag

SEQ ID NO: 461 - DNA, 51 bp, Homo sapiens
  variation (26)..(26): N = C or T
gttgttcatgtccattgattcttggngttcttttactttttccaaaatata

SEQ ID NO: 462 - DNA, 51 bp, Homo sapiens
  variation (26)..(26): N = C or T
gattgaacgtgtgattggcagaaacnggagcccctgcatgcaagacaggag

SEQ ID NO: 463 - DNA, 51 bp, Homo sapiens
  variation (26)..(26): N = A or C or G
tgcacgaggtccagagatacattgancttctccccaccagcctgccccatg

SEQ ID NO: 464 - DNA, 51 bp, Homo sapiens
  variation (26)..(26): N = C or G
tggccctgtgttcactctgtattttngcctgaaacccatagtggtgctgca

SEQ ID NO: 465 - DNA, 51 bp, Homo sapiens
  variation (26)..(26): N = A or C
agcatcatgtttttctgggtcattantacctttttgtatagctcttctgtg

SEQ ID NO: 466 - DNA, 51 bp, Homo sapiens
  variation (26)..(26): N = A or C or T
gtggtgcacgaggtccagagatacantgaccttctccccaccagcctgccc

SEQ ID NO: 467 - DNA, 51 bp, Homo sapiens
  variation (26)..(26): N = A or G or T
aatatcctacagataggtattaaggncatcagcaaatccttaaccaatgta

SEQ ID NO: 468 - DNA, 51 bp, Homo sapiens
  variation (26)..(26): N = C or T
ttatcagctaaagtccaggaagagantgaacgtgtgattggcagaaaccgg

SEQ ID NO: 469 - DNA, 51 bp, Homo sapiens
  variation (26)..(26): N = A or C
tctctcatgtttgcttctcctttcantctggagacagagctctgggagagg

SEQ ID NO: 470 - DNA, 51 bp, Homo sapiens
  variation (26)..(26): N = A or G
cggcgtttctccctcatgacgctgcngaattttgggatggggaagaggagc

SEQ ID NO: 471 - DNA, 51 bp, Homo sapiens
  variation (26)..(26): N = C or T
gcagtgaaggaagccctgattgatcntggagaggagttttctggaagaggc

SEQ ID NO: 472 - DNA, 51 bp, Homo sapiens
  variation (26)..(26): N = A or C or G
ggaaagaaatggaaggagatccggcntttctccctcatgacgctgcggaat

SEQ ID NO: 473 - DNA, 51 bp, Homo sapiens
  variation (26)..(26): N = A or C or T
taaattgtttctaattatttagcctnaccctgtgatcccactttcatcctg

SEQ ID NO: 474 - DNA, 51 bp, Homo sapiens
  variation (26)..(26): N = A or G
tgcagttgacttgtttggagctgggncagagacgacaagcacaaccctgag

SEQ ID NO: 475 - DNA, 51 bp, Homo sapiens
  variation (26)..(26): N = A or C
atttccctgacttctgtgctacatgncaacaaagaatttcccaacccagag

SEQ ID NO: 476 - DNA, 51 bp, Homo sapiens
  variation (26)..(26): N = A or G
gccctacacagatgctgtggtgcacnaggtccagagatacattgaccttct

SEQ ID NO: 477 - DNA, 51 bp, Homo sapiens
  variation (26)..(26): N = A or C
ggagcccctgcatgcaagacaggagncacatgccctacacagatgctgtgg

SEQ ID NO: 478 - DNA, 51 bp, Homo sapiens
  variation (26)..(26): N = G or T
tgtcaccgaggctagacactacctgntgagattatttgagattcctgttct

SEQ ID NO: 479 - DNA, 51 bp, Homo sapiens
  variation (26)..(26): N = A or G
ttaatgggactttgaatagcatttantagcatctcattctgccacttcttt

SEQ ID NO: 480 - DNA, 51 bp, Homo sapiens
  variation (26)..(26): N = A or G
tgtctcttgtttctagggcacaaccntattaatttccctgacttctgtgct

SEQ ID NO: 481 - DNA, 51 bp, Homo sapiens
  variation (26)..(26): N = C or T
aaatctctggttgacccaaagaaccntgacaccactccagttgtcaatgga

SEQ ID NO: 482 - DNA, 51 bp, Homo sapiens
  variation (26)..(26): N = C or G
ttttattcctgacctccattttacanaactttaacctgaaatctctggttg

SEQ ID NO: 483 - DNA, 51 bp, Homo sapiens
  variation (26)..(26): N = C or T
attgtttctaattatttagcctcacnctgtgatcccactttcatcctgggc

SEQ ID NO: 484 - DNA, 51 bp, Homo sapiens
  variation (26)..(26): N = A or G
caccactccagttgtcaatggatttncctctgtgccgcccttctaccagct

SEQ ID NO: 485 - DNA, 51 bp, Homo sapiens; identical to SEQ ID NO: 448
  variation (26)..(26): N = A or G or T

SEQ ID NO: 486 - DNA, 51 bp, Homo sapiens
  variation (26)..(26): N = A or G
attgattgcttcctgatgaaaatggngaaggtaaaatgtaaacaaaagctt

SEQ ID NO: 487 - DNA, 51 bp, Homo sapiens
  variation (26)..(26): N = A or no nucleotide
tgattgcttcctgatgaaaatggagnaggtaaaatgtaaacaaaagcttag

SEQ ID NO: 488 - DNA, 51 bp, Homo sapiens
  variation (26)..(26): N = C or T
gcccttctaccagctgtgcttcattnctgtctgaagaagagcagatggcct

SEQ ID NO: 489 - DNA, 121 bp, Homo sapiens
  variation (61)..(61): N = A or C
tcttgaggctcctttccagctctcagattctgtgatgctcaaagggtgagctctgtgggcncaggacgcatggtagatggagcttagtctttctggtatccagctgggagccaagcacaga

SEQ ID NO: 490 - DNA, 52 bp, Homo sapiens
  variation (27)..(27): N = A or G
tttcccactatcattgattatttcccnggaacccataacaaattacttaaaa

SEQ ID NO: 491 - DNA, 121 bp, Homo sapiens
  variation (61)..(61): N = C or G
acgcatggtggtgctgcatggatatgaagtggtgaaggaagccctgattgatcttggagangagttttctggaagaggccatttcccactggctgaaagagctaacagaggatttggtagg

SEQ ID NO: 492 - DNA, 121 bp, Homo sapiens
  variation (61)..(61): N = A or G
gtgttataaaaagcttggagtgcaagctcacggttgtcttaacaagaggagaaggcttcantggatccttttgtggtccttgtgctctgtctctcatgtttgcttctccyttcamtctgga

SEQ ID NO: 493 - DNA, 121 bp, Homo sapiens
  variation (61)..(61): N = A or G
gcaatttcttaacttgatggaaaaattgaatgaaaacatcaggattgtaagcaccccctgnatccaggtaaggccaagttttttgcttcctgagaaaccacttacagtctttttttctggg

SEQ ID NO: 494 - DNA, 121 bp, Homo sapiens
  variation (61)..(61): N = T or C
cggcgtttctccctcatgacgctgcggaattttgggatggggaagaggagcattgaggacngtgttcaagaggaagcccgctgccttgtggaggagttgagaaaaaccaagggtgggtgac

SEQ ID NO: 495 - DNA, 121 bp, Homo sapiens
  variation (61)..(61): N = C or G
ggctggggactaggtaccccattctagcggggcacagcacaaagctcatagggggatgggntcaccaggaaagcaaagacaccatggtggctgggccggggctgtccagtgggcaccgaga

SEQ ID NO: 496 - DNA, 52 bp, Homo sapiens
  variation (27)..(27): N = T or C
gccccgcacctcagggtccggccacanagctgggttgtgatgggttccgaaa

SEQ ID NO: 497 - DNA, 52 bp, Homo sapiens
  variation (27)..(27): N = A or T
tgaaagataagaaagaactagaaggtnctgggaaggtgagtcaaactaaata

SEQ ID NO: 498 - DNA, 121 bp, Homo sapiens
  variation (61)..(61): N = A or G
ttgatgcttttgaagaacgacakaaaagttggggaattgactgtctttttgaaaagttatntctacttacagaaaagtaaatgagacatagataaaataaaatcacactgacatgtttttg

SEQ ID NO: 499 - DNA, 121 bp, Homo sapiens
  variation (61)..(61): N = T or C
taaacaaaaaaagaaaaattacttaccatttgcgatcacctggattgatggcaactaatgntcctctatcccaaatcatgtcaaatttgccaatatttgtcctaccagaaagagaaaaaac

SEQ ID NO: 500 - DNA, 52 bp, Homo sapiens
  variation (27)..(27): N = C or G
gtaaatgtatgattttatgcaggtttncagaccggggacacagtgtagttgg

SEQ ID NO: 501 - DNA, 121 bp, Homo sapiens
  variation (61)..(61): N = A or G
gtagagacggggtttcaccatattggccaggctggtctcaaactcctgacctcatgatccnccctccttggcttcctaaagtgctgggattacaggcgtgagccactgtgcctggcctttt

SEQ ID NO: 502 - DNA, 52 bp, Homo sapiens
  variation (27)..(27): N = A or G
ggattataggcgtgagccaccgcaccnggccaatggttgtttttcaggtctt

SEQ ID NO: 503 - DNA, 121 bp, Homo sapiens
  variation (61)..(61): N = T or C
tctgtactgggctctgaccacaatcggttttcagaccacaatgttaggagggtatttttanatccctccagttaacaaatacagcactggcatggttcaccttctcctgcaggtgaccatt

SEQ ID NO: 504 - DNA, 121 bp, Homo sapiens
  variation (61)..(61): N = A or G
acaatacagatctggtcgagtttaaaactctcactgaggaagaggttgaagaagtgctganaaatatatttaagatttccttggggagaaatctcgtgcccaaacctggtgatggatccct

SEQ ID NO: 505 - DNA, 121 bp, Homo sapiens
  variation (61)..(61): N = A or G
attctcatctcctgccaaagaagaaacaccaaaaaatatacttatttacgcttgaacctcnaacaattgaagattttgagtctatgaatacatacctgcagacgtctccaacatcttcatt

SEQ ID NO: 506 - DNA, 121 bp, Homo sapiens
  variation (61)..(61): N = A or G
tgctgagaaatatatttaagatttccttggggagaaatctcgtgcccaaacctggtgatgnatcccttactatttagaataaggaacaaaataaacccttgtgtatgtatcacccaactca

SEQ ID NO: 507 - DNA, 121 bp, Homo sapiens
  variation (61)..(61): N = A or G
gtgggcaagccatggagttgggcttagaggctatttttgatcacattgtaagaagaaacyngggtgggtggtgtctccaggtcaatcaacttctgtactgggctctgaccacaatcggttt

SEQ ID NO: 508 - DNA, 121 bp, Homo sapiens
  variation (61)..(61): N = T or C
acatccctccagttaacaaatacagcactggcatggttcaccttctcctgcaggtgaccantgacggcaggaattacattgtcgatgctgggtctggaagctcctcccagatgtggcagcc

SEQ ID NO: 509 - DNA, 121 bp, Homo sapiens
  variation (61)..(61): N = A or G
ggaactctaacacctccggtgagttctcaccttaccttttgttagagaggagttgggaccnttcatgctgggagtcagatgtctggagaaatggcttcgtgtgatcgagtgaattcactgc

SEQ ID NO: 510 - DNA, 121 bp, Homo sapiens
  variation (61)..(61): N = A or G
gcaaaatgtttaattcagtgatgttcttacagttacaggtattctaaagaaactaatatcnattcatcagaaaattcaacatcgaccttatccacttgtttaattaatcaaattttatcac

SEQ ID NO: 511 - DNA, 121 bp, Homo sapiens
  variation (61)..(61): N = T or C
aacactctcttatctacataggttgtttaaaggaatctgggtcatacatgtggatatatgngttcatgggtaatatgcttcgtggaataggggagactcccatagtaccaytggggctttc

(19) United States
(12) Patent Application Publication
(10) Pub. No.: US 2017/0137968 A1
(43) Pub. Date: May 18, 2017

(54) Method and System for Diagnosing Disease and Generating Treatment Recommendations

(71) Applicant: Global Gene Corporation Pte. Ltd., Singapore (SG)

(72) Inventors: Saumya Jamuar, Singapore (SG); Jonathan Picker, Boston, MA (US); Shalendra Porwal, Columbus, OH (US); Kushagra Sharma, Mumbai (IN); Sumit Jamuar, London (GB)

(73) Assignee: Global Gene Corporation Pte. Ltd., Singapore (SG)

(21) Appl. No.: 15/258,355

(22) Filed: Sep. 7, 2016

Related U.S. Application Data
(60) Provisional application No. 62/215,046, filed on Sep. 7, 2015.
(60) Provisional application No. 62/215,047, filed on Sep. 7, 2015.
(60) Provisional application No. 62/215,048, filed on Sep. 7, 2015.
(60) Provisional application No. 62/215,049, filed on Sep. 7, 2015.
(60) Provisional application No. 62/243,150, filed on Oct. 19, 2015.
(60) Provisional application No. 62/363,776, filed on Jul. 18, 2016.

(30) Foreign Application Priority Data
Sep. 11, 2015 (IN) 3484/MUM/2015

(51) Int. Cl. C40B 30/02; C12Q 1/68

(52) U.S. Cl. CPC C40B 30/02; C12Q 1/6886; C12Q 1/6883; C12Q 1/6837; C12Q 2600/106; C12Q 2600/118; C12Q 2600/158; C12Q 2600/156; C12Q 2600/16

(57) ABSTRACT

The present invention relates generally to methods, algorithms, kits and systems for assessing health, diagnosing disease and generating recommendations using SNV markers specific to a cohort. A genetic sample of an individual is assayed using a genotyping assay to identify at least one SNV. The genotyping assay may be a computer analysis using a database, a nucleic acid microarray assay or a PCR assay. The identified SNV can be compared with a database of SNV markers to identify a plurality of risk SNVs, which are associated with a disease state or pathological condition, including pharmacological sensitivity or resistance. A genetic risk factor (GRF) may be calculated using a weighted score. The GRF is used to determine the risk level associated with the disease. A matrix may be generated using the genetic profile and recommendations specific to cohort and physiologic data. The user is allowed to input physiologic and genomic data, which is compared to the matrix to generate recommendations. In another aspect, the present invention relates to an analytical tool to analyze and relate genomic data with an individual's phenotype across multiple dimensions such as his or her health, age, family, ethnicity, environment and current scientific understanding. The analytical tool enables the individual to specify the genomic sequence as well as to feed in his or her phenotype data along with his or her family's phenotype data. The genomic sequence entered is then compared with a population database to generate a list of associated genetic disorders. This list is then overlaid against the individual's phenotype and his or her family phenotype data to confirm the genetic disorders identified. A real time report is generated and data is updated in real time on the population database to provide relevant and updated genetic information to users.

### PRIORITY CLAIM

The present application claims priority to the following applications, each hereby incorporated by reference in their entirety: (1) Cardiochip, U.S. Ser. No. 62/215,046, filed Sep. 7, 2015; (2) IndiaDIABETESchip, U.S. Ser. No. 62/215,047, filed Sep. 7, 2015; (3) IndiaGENETICchip, U.S. Ser. No. 62/215,048, filed Sep. 7, 2015; (4) Pharmachip, U.S. Ser. No. 62/215,049, filed Sep. 7, 2015; (5) PharmaDB, Indian Patent Application No. 3484/MUM/2015, filed Sep. 11, 2015; (6) IP Genomic Analyzer, U.S. Ser. No. 62/243,150, filed Oct. 19, 2015; and (7) SNP Markers, U.S. Ser. No. 62/363,776, filed Jul. 18, 2016.

### BACKGROUND OF THE INVENTION

Field of the Invention

The present invention relates to methods, algorithms, kits and a system for generating health assessments, diagnoses, disease treatment and/or disease prevention recommendations using single nucleotide variants (SNV) markers specific to a cohort. More particularly, the present invention relates to methods, algorithms and a system for analyzing disease risk genes associated with a selected cohort, demographic population, ethnic group, and/or national origin, and providing an assessment or diagnoses and recommendations based on individuals' genetic, genomic, lifestyle, and/or physiologic data.

Description of the Related Art

The genetic variations in a DNA sequence due to a point mutation, deletion, insertion or small polymorphism in a genome are called single nucleotide variants, or SNVs. SNVs occur normally throughout an individual's DNA and account for the differences in a genetic makeup of two individuals of a particular biological species. The genetic variations represented by the SNVs result in differences in the characteristics and traits of individuals. The genetic variations may correlate to development of a disease or a response of the individual towards various external agents, such as drugs, pathogens, chemicals, and environmental conditions. Thus, the SNVs act as biological markers for locating sequences in genes that are associated with a particular pathological condition or drug response. In addition to that, the SNVs also provide an assessment of a risk associated with the genes for developing the pathological condition.

When a pathological condition is specific to a particular cohort, the SNVs that correlate to the pathological condition are also specific to that cohort, since the SNVs have also evolved under similar environmental conditions. Hence, analysis of the SNVs that are more prominently found in a particular cohort provides a comprehensive approach for assessment and/or diagnosis of the particular pathological condition.

Accurate determination of an individual's risk of developing a pathological condition is a challenging task. Determining the risk of developing a pathological condition involves the calculation of genetic risk factor based on the patient's genomic information, among other factors. Various algorithm and methods are developed for the calculation of genetic risk factor associated to a particular pathological condition.

In one example, U.S. Patent Publication No. 2011/0202486 discloses a method and system for predicting development of a cardiovascular condition of interest in a patient. The patient's genetic data and non-genetic data are used to calculate a risk score. The calculated risk score is used to determine the risk level. Further, a preventive strategy is suggested, based on the risk level. However, the disclosed method fails to consider the selected cohort of SNVs while calculating the risk score. Moreover, the system generates a treatment strategy but not preventive recommendations, based on daily developments in an individual's lifestyle.

With advancement in genetic technologies, thousands of SNVs are being identified more readily. However, their relevance to the health of an individual remains to be defined. Thus, the development of a consolidated approach for calculating the genetic risk factor associated with various pathological conditions across one or more cohorts and generating corresponding health care options is needed, often in combination with physiologic data. A need persists to develop such a customized method that utilizes a cohort-associated SNV disease related database to assess risk. Also needed is an algorithm which most accurately estimates the risk, provides diagnosis, and generates corresponding healthcare options for the individual.

A genome is the entire set of hereditary instructions for building, running and maintaining an organism, and passing life on to the next generation. A genotype is a unique genome that is revealed by genome sequencing. However, the word genotype can also refer to an individual's particular gene or set of genes. Genome sequencing is an important step in understanding the genome. A genome sequence helps to determine a gene and gene variants easily and quickly.

In many cases, gene variants are associated with observable physical characteristics or traits such as morphology, development, biochemical or physiological properties, and behaviour. Such observable physical characteristics or traits are referred to as phenotypes. These include straightforward visible characteristics such as an individual's height and eye color as well as behavior and general disposition. Studies have revealed that a specific genetic disorder is identified through an associated specific phenotype. However, not all phenotypes are the direct result of a genotype. Phenotypes are influenced both by a genotype and the unique circumstances in which an individual lives his or her life

Sequencing a person's genome has found clinical applications, particularly in diagnosis of rare childhood conditions and cancer therapeutics. Moreover, application of genotype data along with phenotype data enhances the ability to make informed and appropriate decisions relating to health care, including, for example, treatment of specific diseases, and choice of drugs and drug dosage. Over the years, several analytical tools have been developed to determine genome sequences and compare these with existing medical databases to identify genetic disorders. Several other existing tools help to diagnose genetic disorders by searching for keywords related to phenotype data in database. However, consolidated analytical tools integrating genotype and phenotype data are not widely known because of the complexity in associating genotype and phenotype data. This complexity arises due to the varying associations of genotype and phenotype data in individuals, since this depends on their genomes, ages, families, lifestyles, habits, personal health, and environmental and demographic factors.

In some cases, the presence of a particular genome in an individual may or may not lead to a predicted phenotype and genetic disorder. Thus, direct mapping of the individual's genetic profile to predict the onset of corresponding genetic disorders is not completely accurate. Such genetic disorders mainly depend on data on the individual's ancestry. Therefore, accurate genetic disorders may be predicted by analyzing genetic disorders in the individual's family in the past. However, the enormous size and complexity of the database leads to several theoretical and statistical challenges, including real-time updates with data management and profiling.

To overcome the drawbacks mentioned above, US patent application 2002/0052761 discloses a system for generating individual-specific personal health reports by analyzing a set of genomes and phenotype data. However, the system diagnoses a particular genetic disorder, based on the demographic population, but does not consider the individual's family history. Moreover, the system fails to update database in real time.

Therefore, given the facts given above, there is a need to develop a robust analytical tool to predict accurate genetic disorders, based on an individual and his or her family's genetic and phenotype data, and also updates the database in real time.

### SUMMARY OF THE INVENTION

An object of the present invention is to provide methods, algorithms, kits and a system for assessing or diagnosing a disease in a person specific to a particular cohort using single nucleotide variant (SNV) markers. Also included are SNV microarrays, PCR primers for amplification of SNV containing nucleic acids, and kits for diagnostic methods.

Another object of the present invention is to provide a method of diagnosing a person for a disease by calculating a genetic risk factor (GRF).

Embodiments of the present invention provide a method for identification of treatable genetic profiles of a person using single nucleotide variant (SNV) markers. The method involves creating a database of single nucleotide variant (SNV) markers which are specific to one or more cohorts and have a high association with the pathological condition. In the next step, a genetic sample of the person is assayed using genotyping assay to identify a plurality of SNVs. The plurality of SNVs is then compared with the database of SNV markers to identify a plurality of risk SNVs which are associated with the pathological condition. Then, a weighted score is provided to the plurality of risk SNVs based on odds ratio corresponding to each risk SNV. A genetic risk factor (GRF) is calculated using the weighted score. The GRF is then compared with a plurality of set of ranges to provide a preventive healthcare recommendation.

The method further involves creation of a genetic profile for an individual based on the identified disorders and several influencing physiological factors. A matrix is generated by retrieving data from the database of healthcare-related recommendations for the identified condition and physiological factors. The recommendations are then generated by mapping one or more physiologic conditions in the generated matrix. Further, the recommendations are generated based on the identified cohort specific disorder and the mapped physiological conditions.

Another object of the present invention is to provide a genomic analyzer to analyze and relate genomic data with phenotype data across multiple dimensions such as health, age, family, ethnicity, and environment.

Another object of the present invention is to provide a genomic analyzer that will predict the most relevant and accurate genetic disorders as well as associated phenotype data, based on individual and his or her family's genotype and phenotype data.

Yet another object of the present invention is to provide a genomic analyzer that will update genotype and phenotype data in real time to population data.

Embodiments of the present invention provide a method to relate the genomic data of an individual with that of his or her family and as well as to the population to which he or she belongs, and analyze this data in relation to his or her clinical presentation. The method involves the creation of population data specific to a demographic region. The method also involves receiving an individual's genomic and phenotype data as well as his or her family's genomic and phenotype data. A genome sequence entered by the individual is compared with the population data to generate a list of gene variants and associated phenotype data. Further, the list of gene variants and the associated phenotype data are compared with the individual's phenotype data to confirm the associated phenotype. The unconfirmed phenotype data is then compared with the phenotype data of the individual's family to accurately generate updated individual-specific genomic-phenotype association data. This updated genomic-phenotype association data is fed in the population data.

Related to another aspect of the invention, the genome is the entire set of hereditary instructions for building, running and maintaining an organism, and passing life on to the next generation. A genotype is a unique genome that is revealed by genome sequencing. However, the word genotype can also refer to an individual's particular gene or set of genes. Genome sequencing is an important step in understanding the genome. A genome sequence helps to determine a gene and gene variants easily and quickly.

In many cases, gene variants are associated with observable physical characteristics or traits such as morphology, development, biochemical or physiological properties, and behaviour. Such observable physical characteristics or traits are referred to as phenotypes. These include straightforward visible characteristics such as an individual's height and eye color as well as behavior and general disposition. Studies have revealed that a specific genetic disorder is identified through an associated specific phenotype. However, not all phenotypes are the direct result of a genotype. Phenotypes are influenced both by a genotype and the unique circumstances in which an individual lives his or her life

Sequencing a person's genome has found clinical applications, particularly in diagnosis of rare childhood conditions and cancer therapeutics. Moreover, application of genotype data along with phenotype data enhances the ability to make informed and appropriate decisions relating to health care, including, for example, treatment of specific diseases, and choice of drugs and drug dosage. Over the years, several analytical tools have been developed to determine genome sequences and compare these with existing medical databases to identify genetic disorders. Several other existing tools help to diagnose genetic disorders by searching for keywords related to phenotype data in database. However, consolidated analytical tools integrating genotype and phenotype data are not widely known because of the complexity in associating genotype and phenotype data. This complexity arises due to the varying associations of genotype and phenotype data in individuals, since this depends on their genomes, ages, families, lifestyles, habits, personal health, and environmental and demographic factors.

In some cases, the presence of a particular genome in an individual may or may not lead to a predicted phenotype and genetic disorder. Thus, direct mapping of the individual's genetic profile to predict the onset of corresponding genetic disorders is not completely accurate. Such genetic disorders mainly depend on data on the individual's ancestry. Therefore, accurate genetic disorders may be predicted by analyzing genetic disorders in the individual's family in the past. However, the enormous size and complexity of the database leads to several theoretical and statistical challenges, including real-time updates with data management and profiling.

To overcome the drawbacks mentioned above, US patent application 2002/0052761 discloses a system for generating individual-specific personal health reports by analyzing a set of genomes and phenotype data. However, the system diagnoses a particular genetic disorder, based on the demographic population, but does not consider the individual's family history. Moreover, the system fails to update database in real time.

Therefore, given the facts given above, there is a need to develop a robust analytical tool to predict accurate genetic disorders, based on an individual and his or her family's genetic and phenotype data, and also updates the database in real time.

### BRIEF DESCRIPTION OF DRAWINGS

FIG. 1 shows a block diagram of a system for health assessment, diagnosing disease and/or generating recommendations using SNV markers specific to a particular cohort, in accordance with an embodiment of the present invention; and

FIGS. 2A and 2B are a flowchart showing the steps involved in a method for diagnosing disease and/or generating the recommendations using SNV markers specific to a particular cohort, in accordance with an embodiment of the present invention.

FIGS. 3A and 3B list SNV-containing nucleic acids, which SNVs are specific to a disease state, the SNVs forming the database or array of SNV markers specific for a demographic segment. The numbered gene names and loci in 3A correspond to the numbered nucleic acid sequences in 3B.

FIG. 4 illustrates a block diagram of a computer system to implement a genomic analyzer, according to an embodiment of the invention.

FIGS. 5A and 5B are a flowchart illustrating the steps involved in a method that analyzes and relates genomic data with phenotype data of an individual, according to an embodiment of the invention.

### DEFINITIONS

The present invention provides single nucleotide variants which are associated with a risk for developing a pathological condition, wherein the SNVs are specific for a particular cohort. These SNVs can be used in health assessments, diagnostic methods and systems, either in a computer database, as a nucleic acid microarray on a chip, or as a template for PCR amplification.

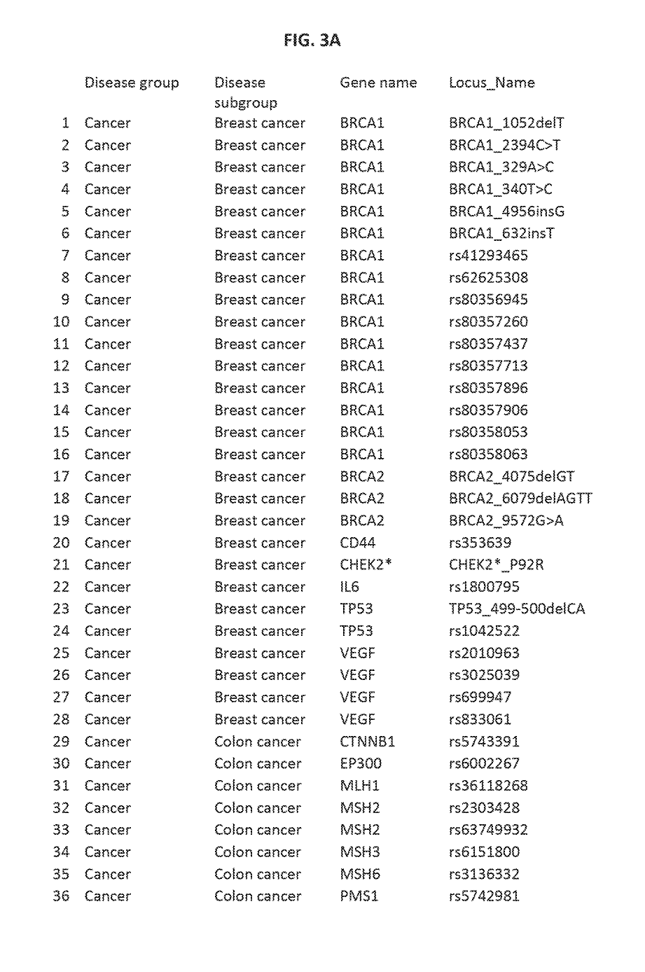

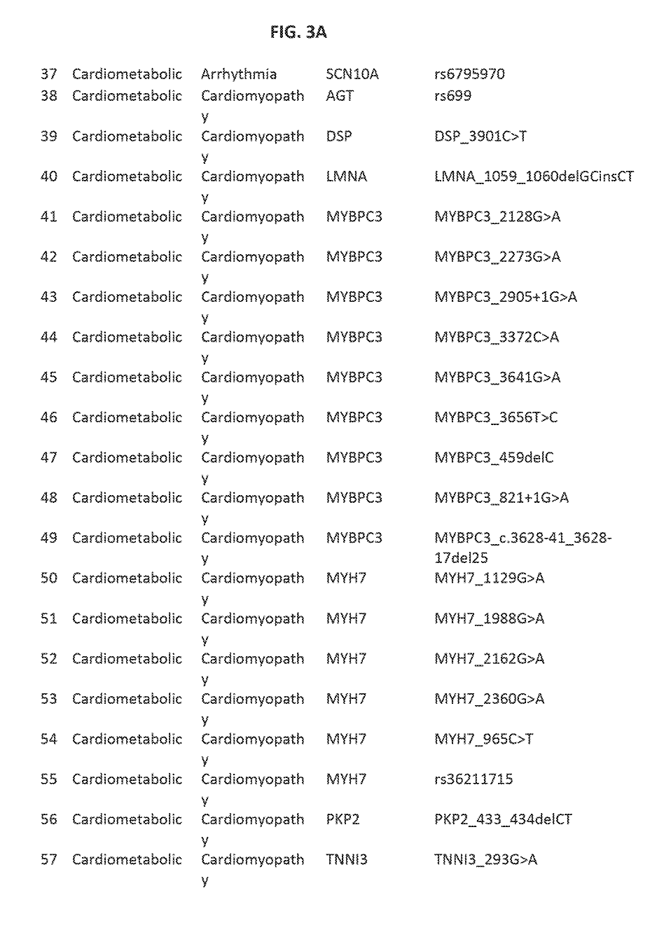

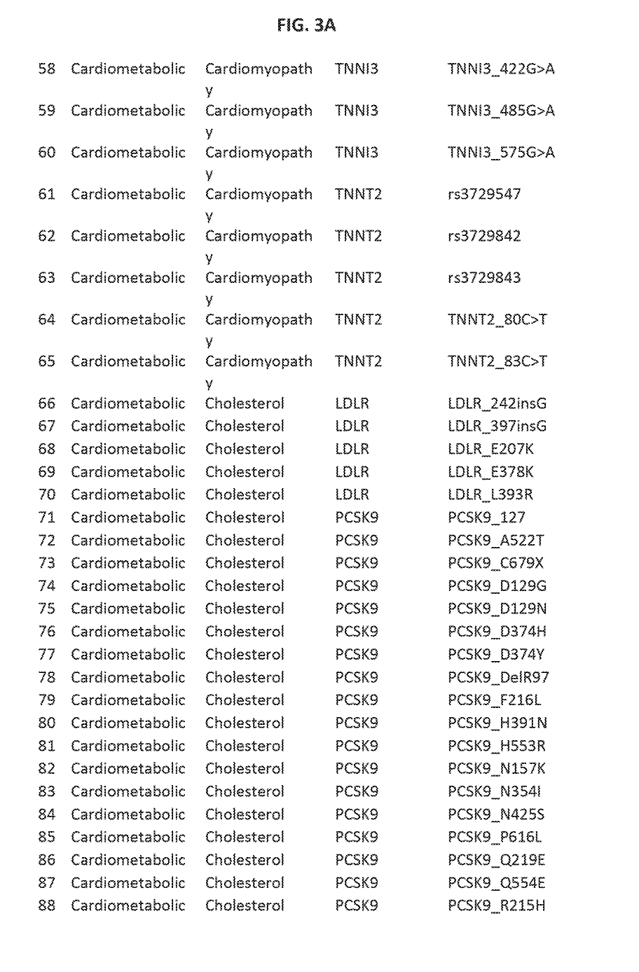

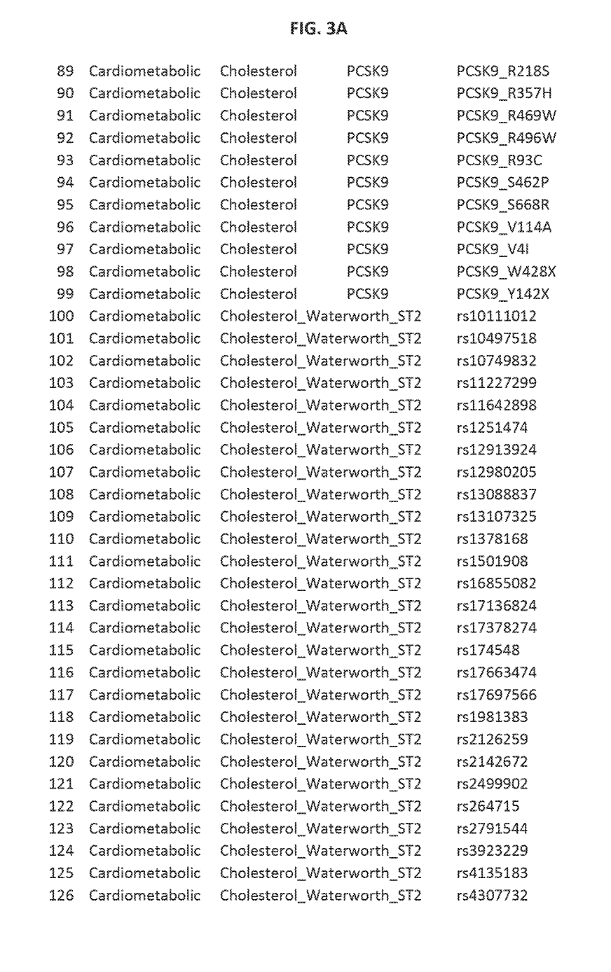

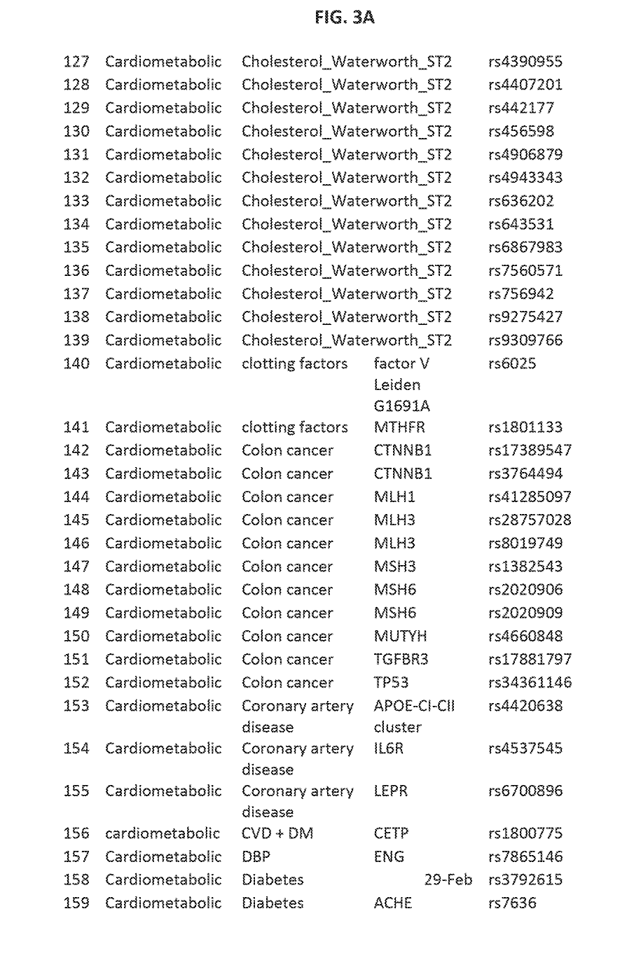

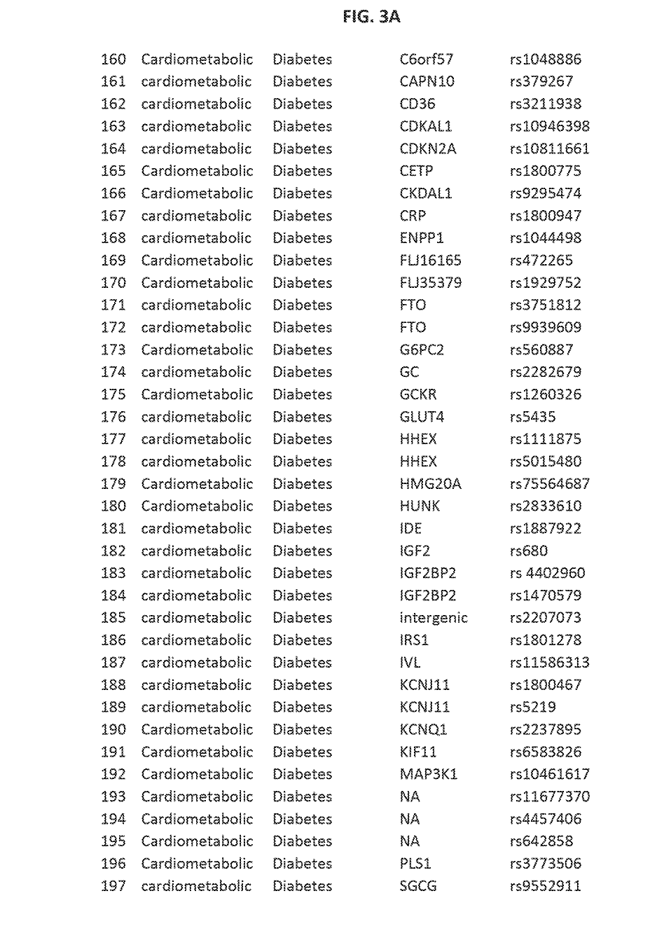

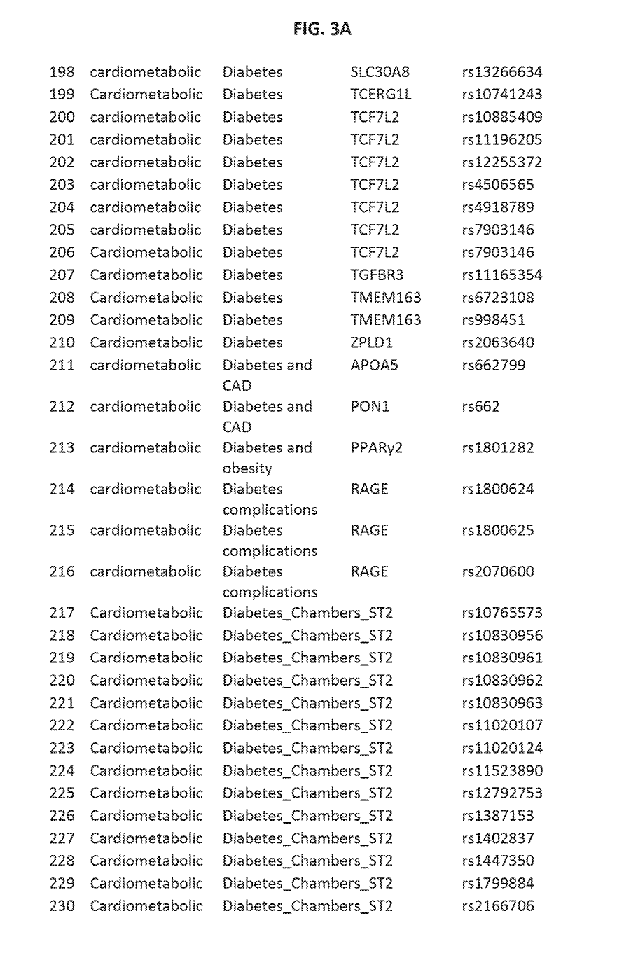

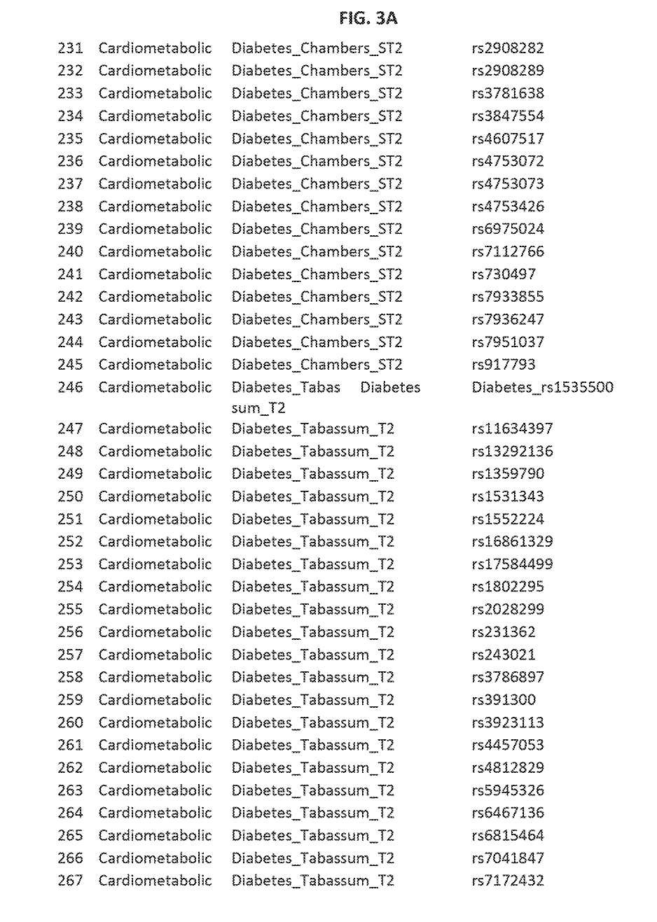

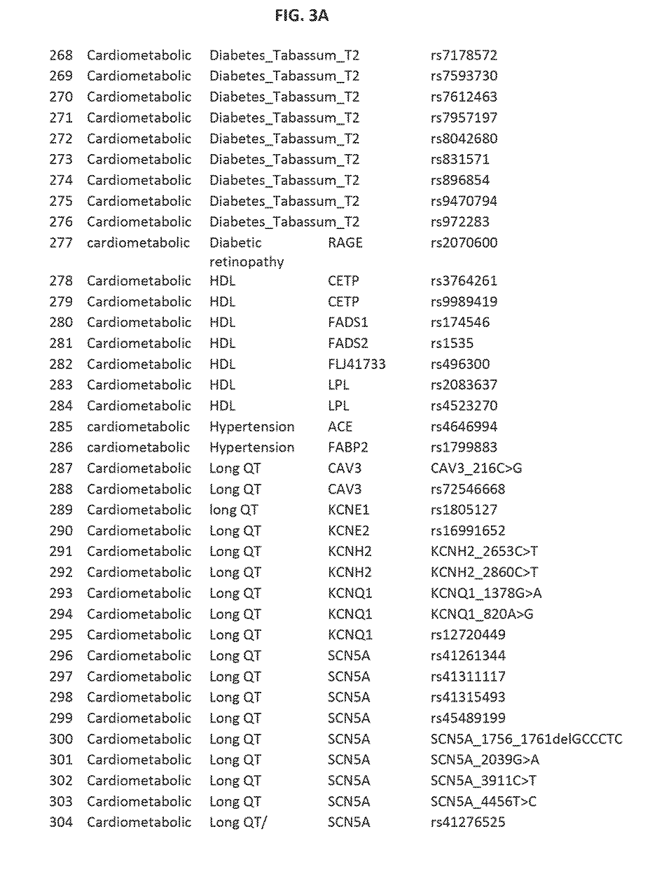

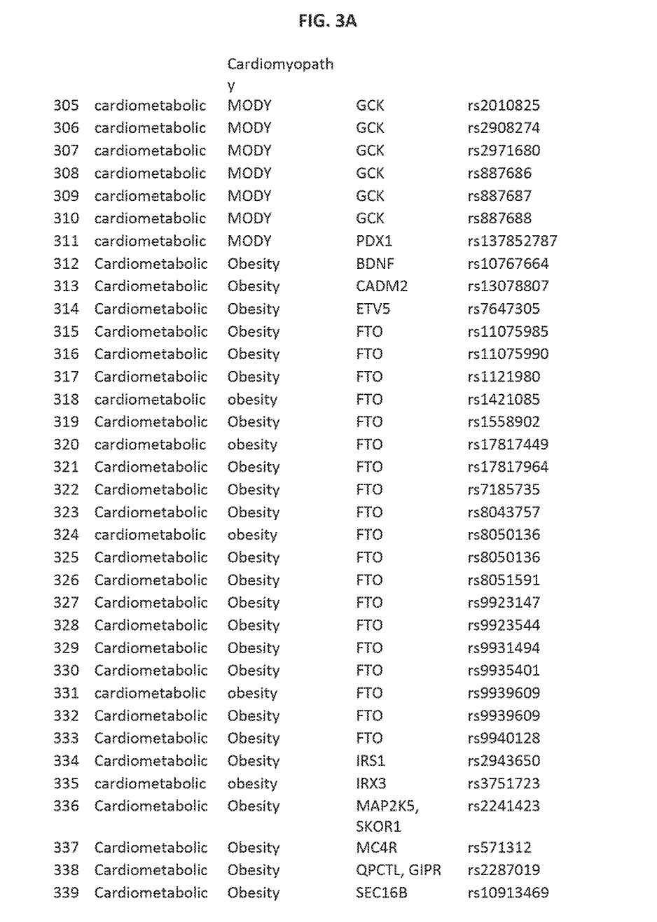

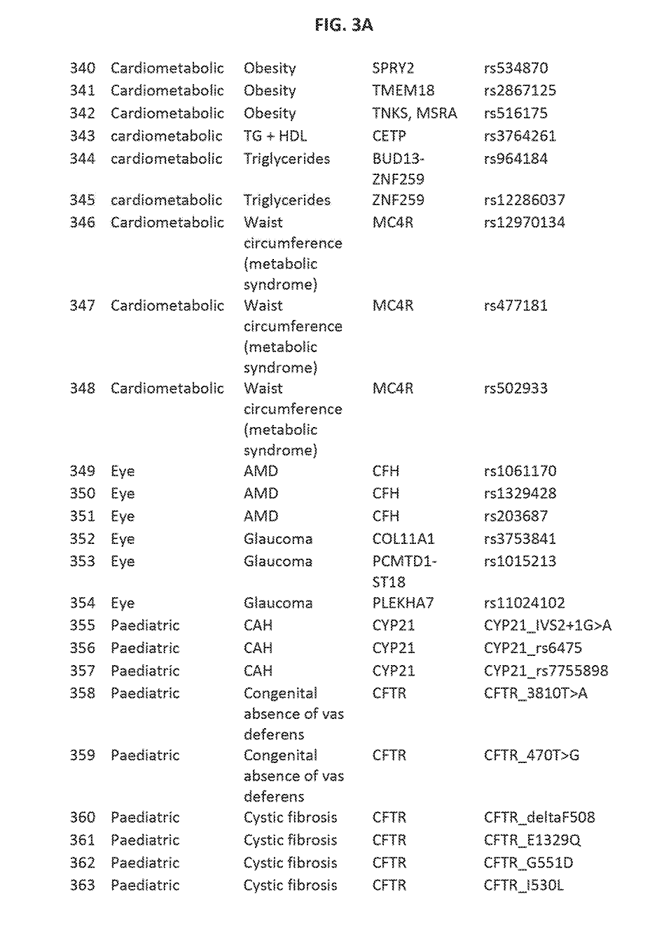

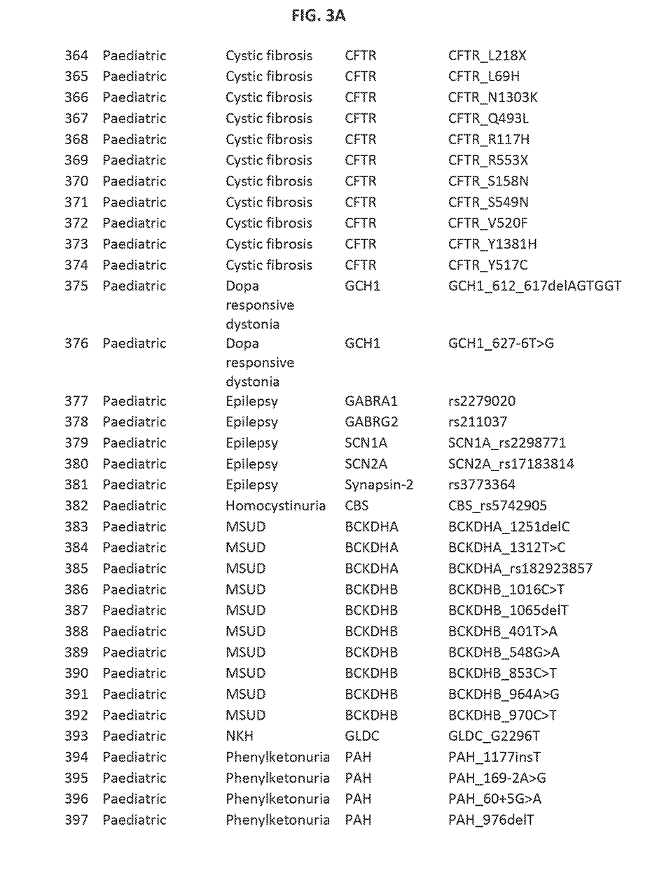

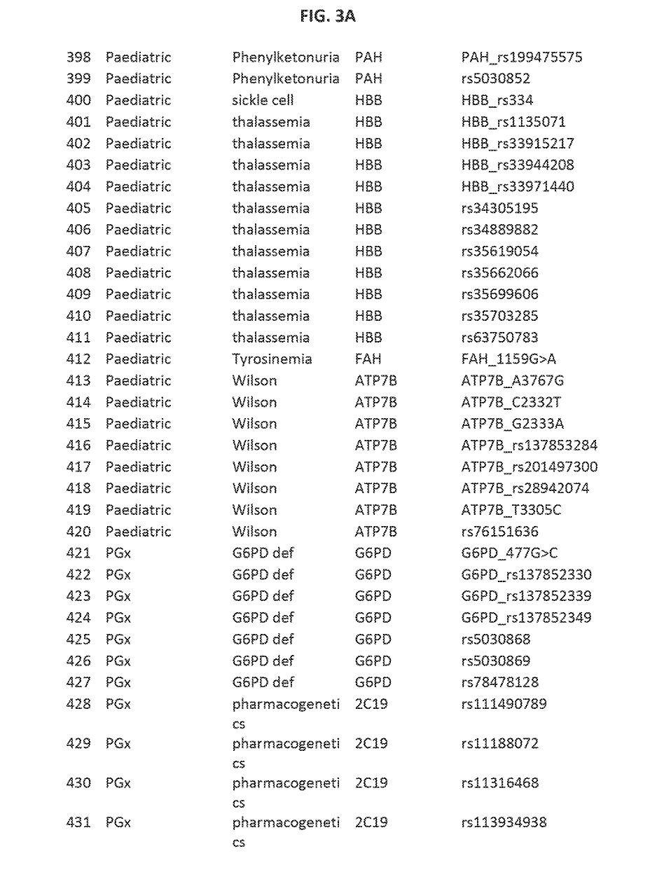

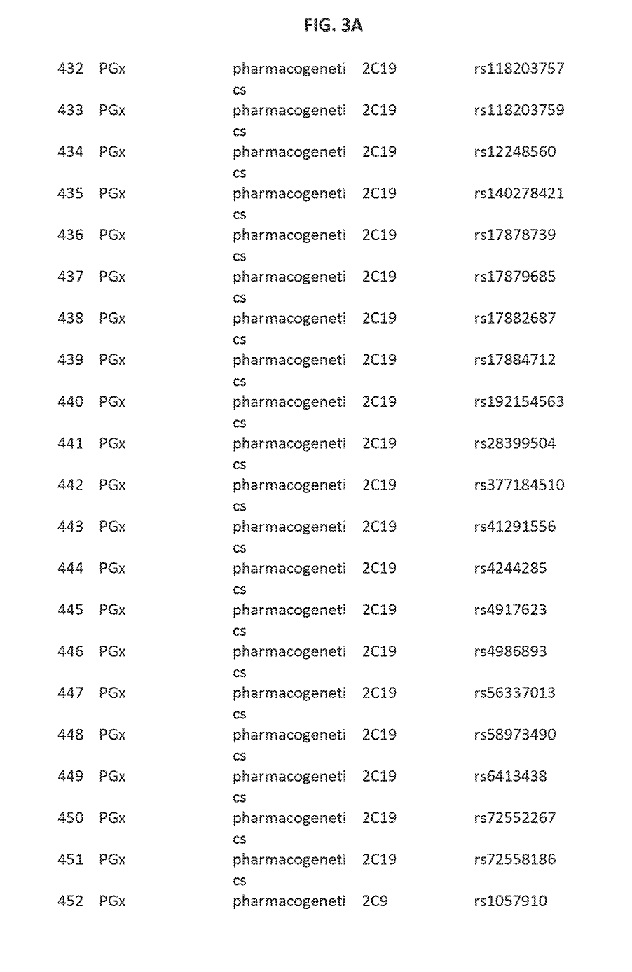

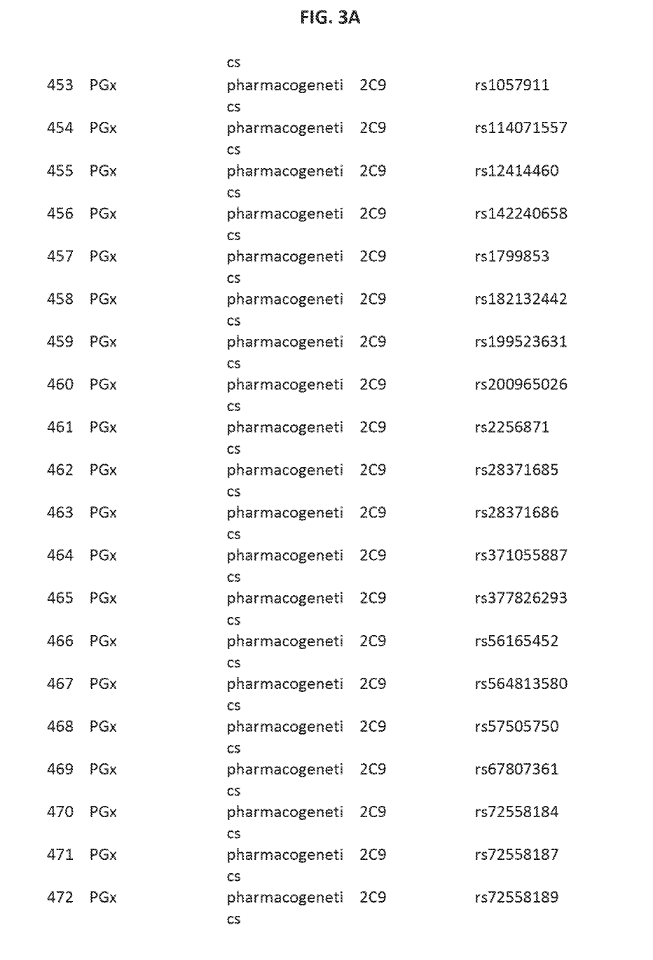

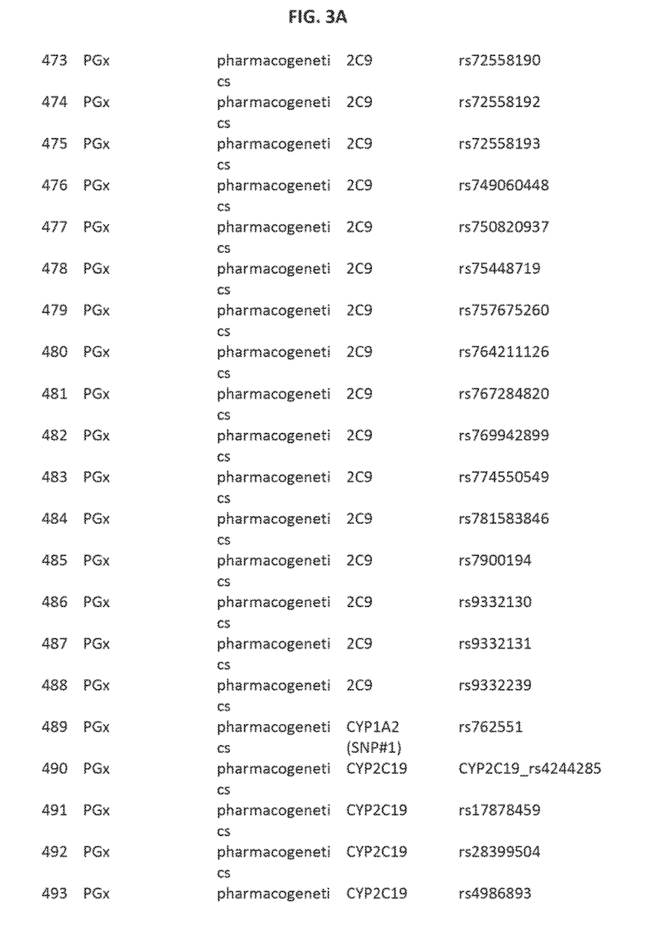

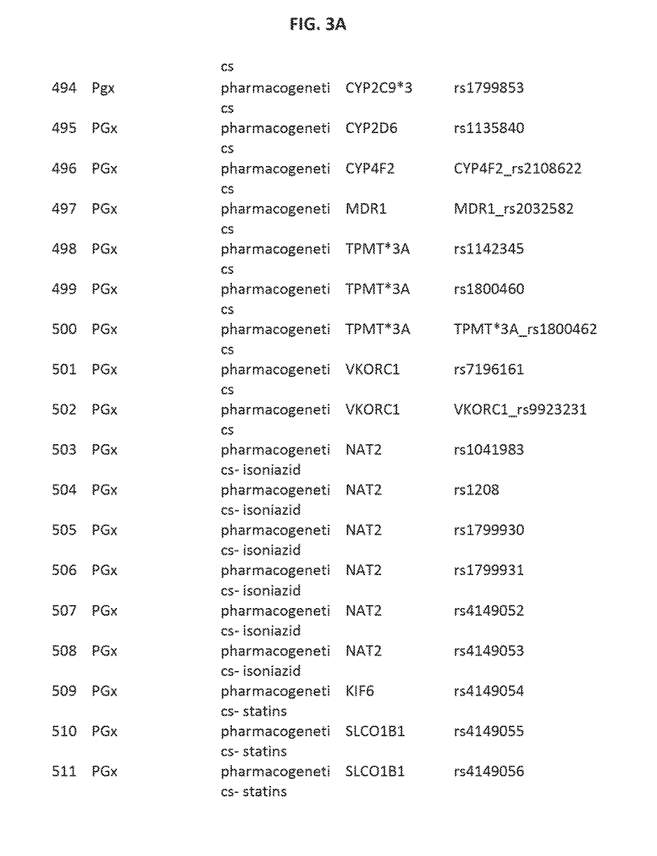

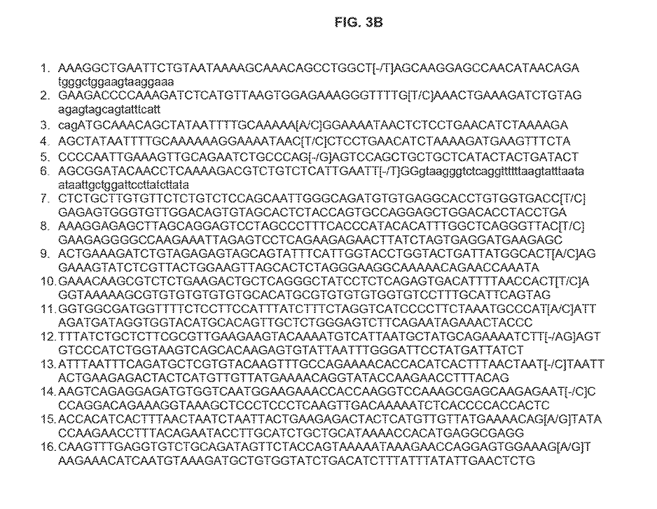

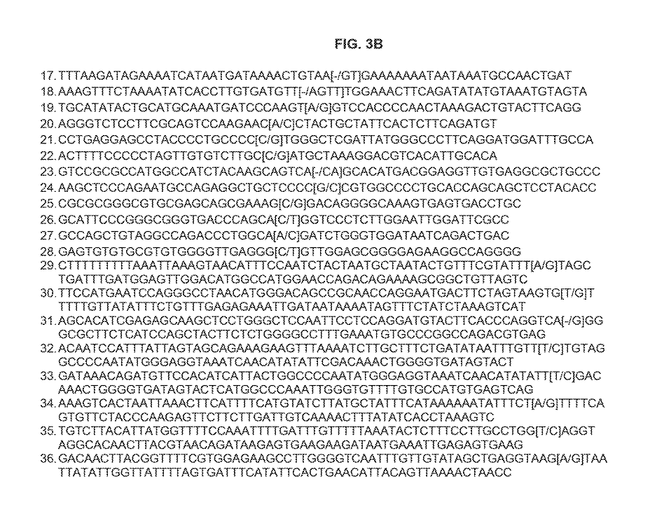

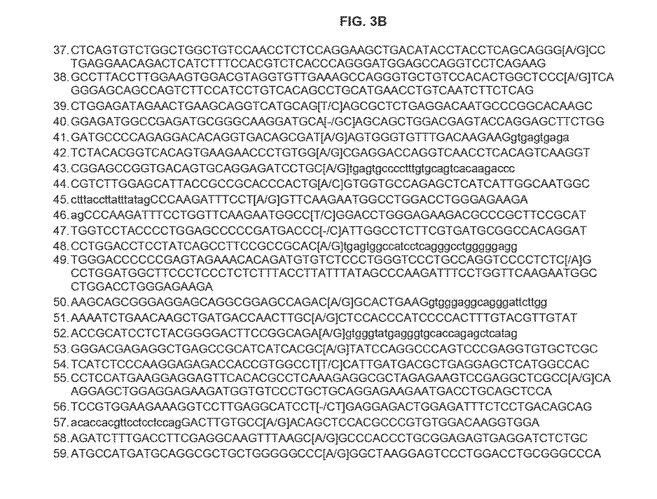

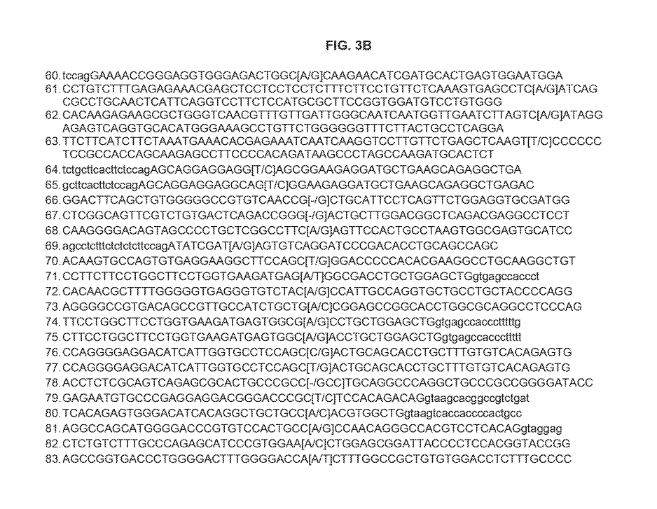

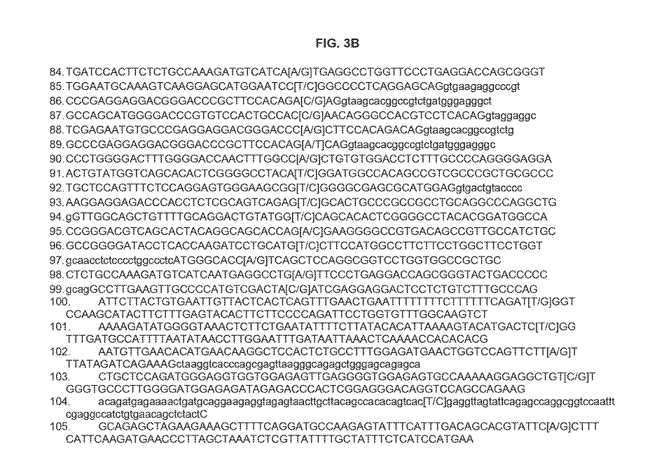

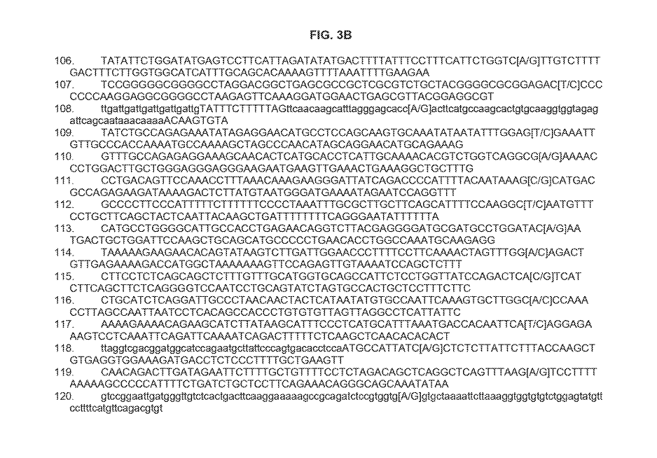

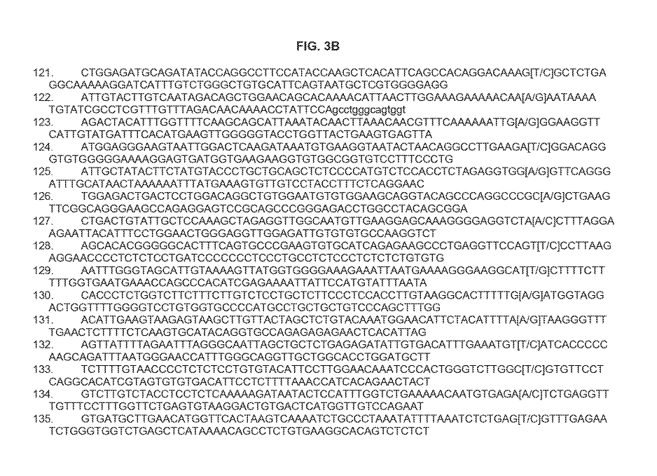

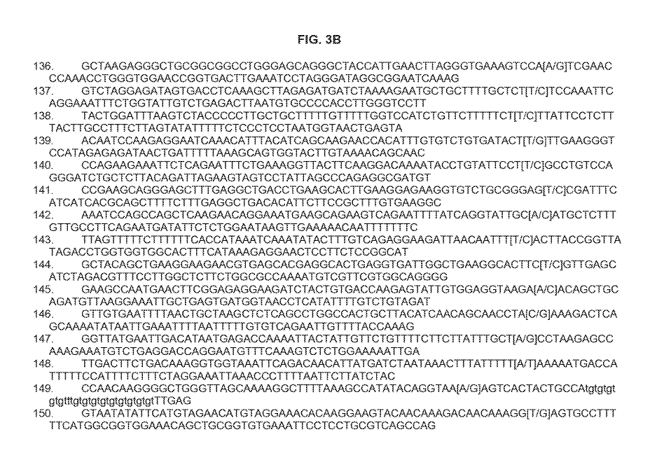

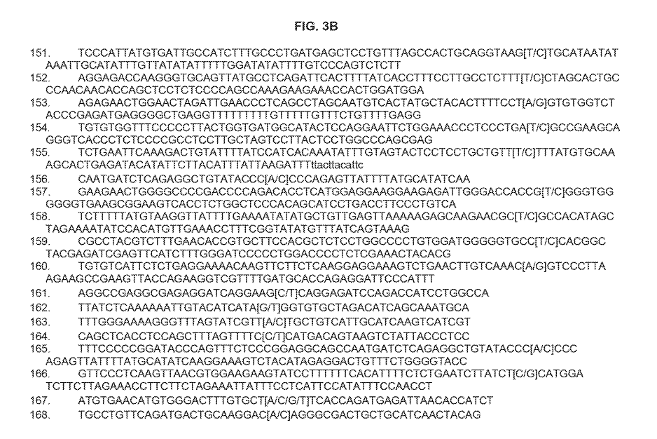

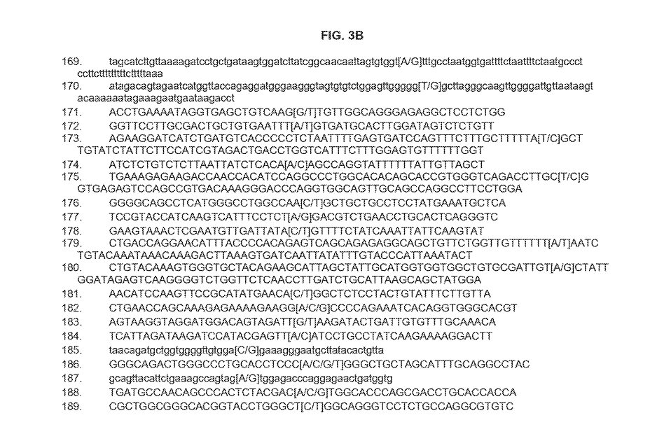

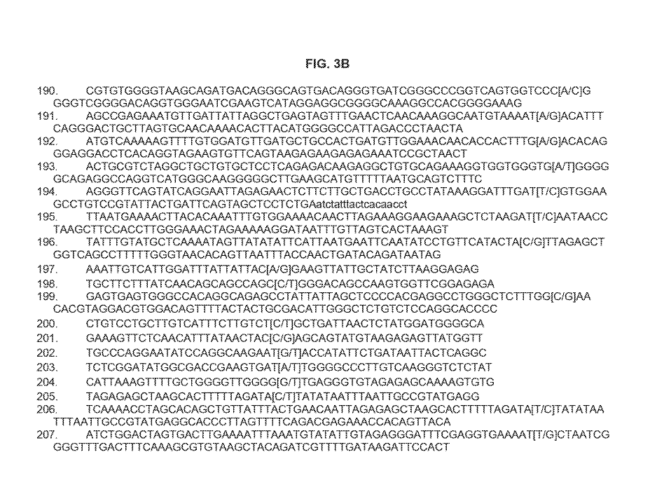

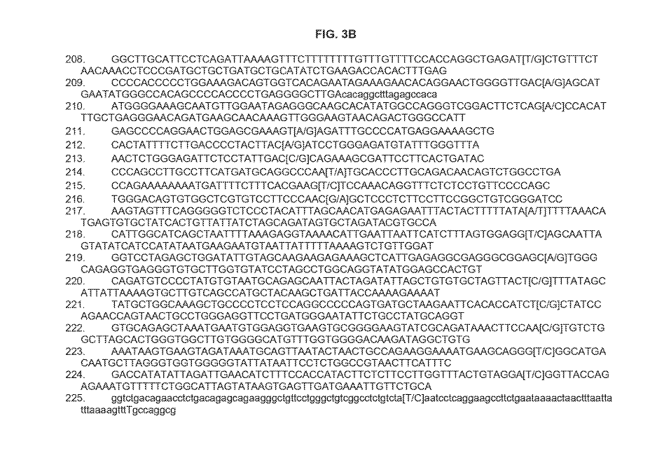

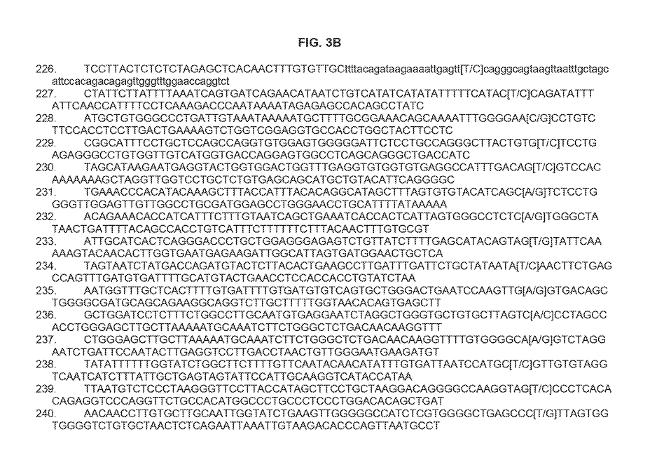

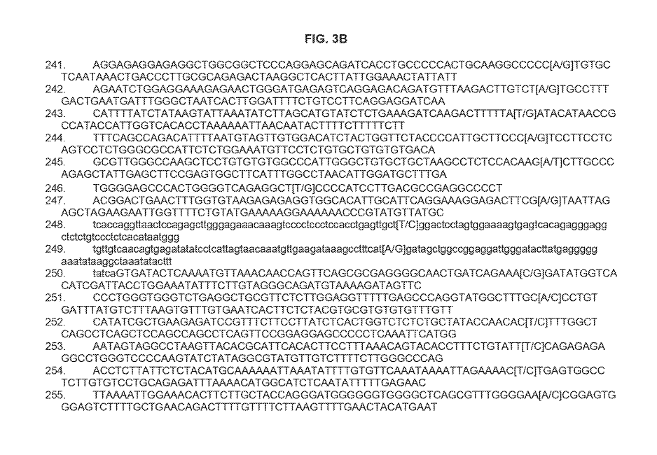

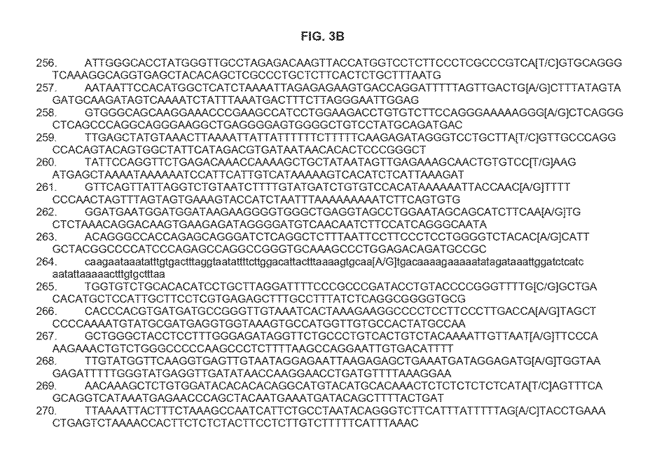

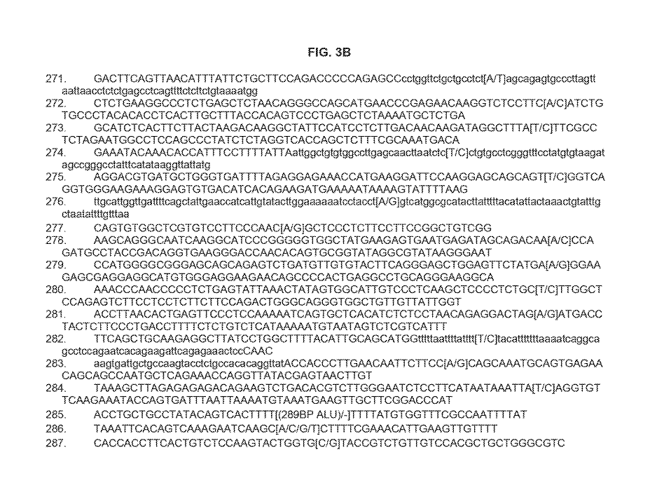

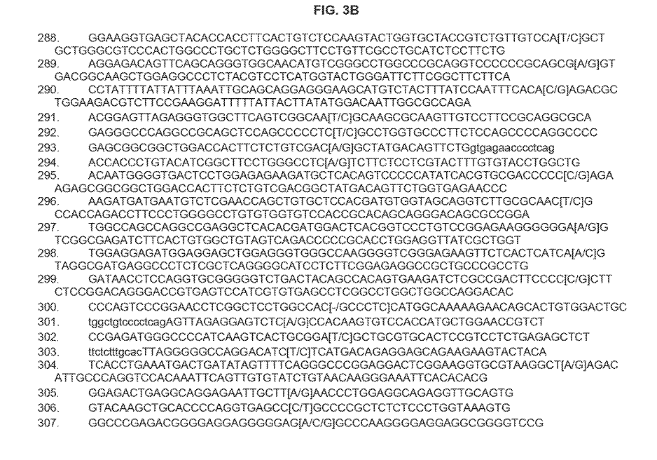

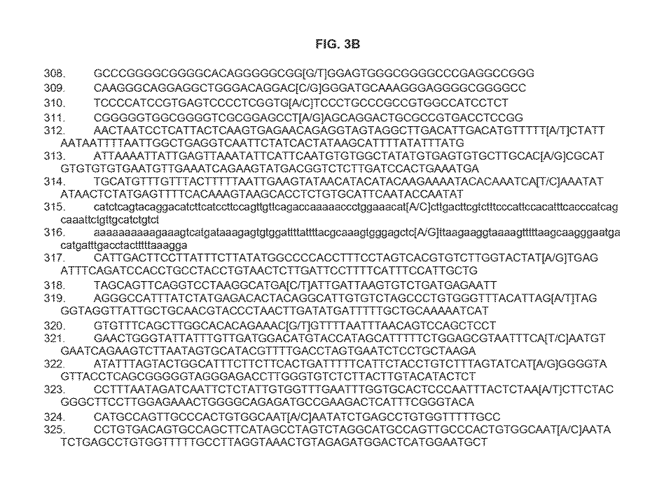

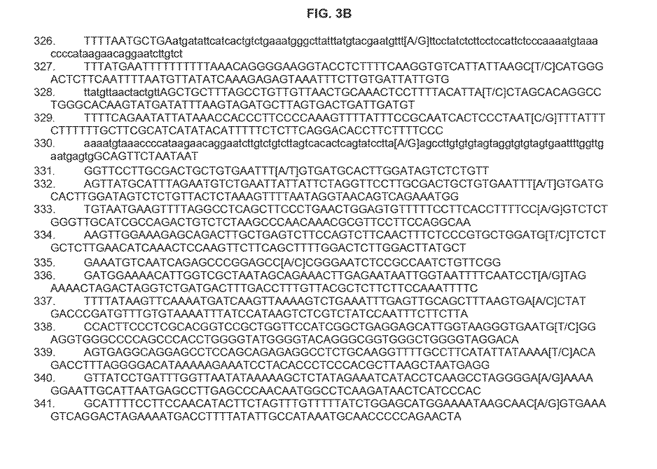

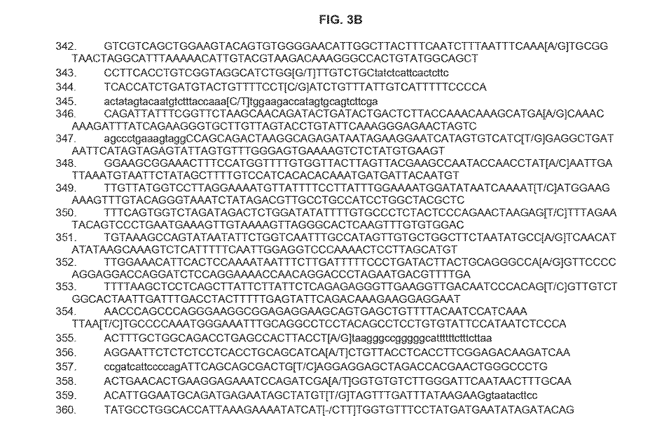

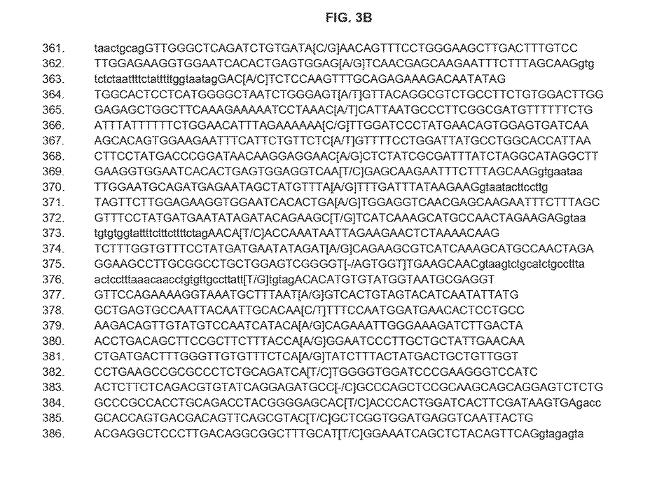

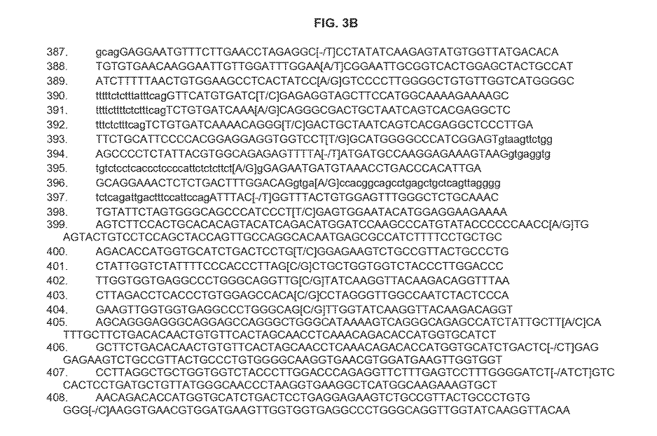

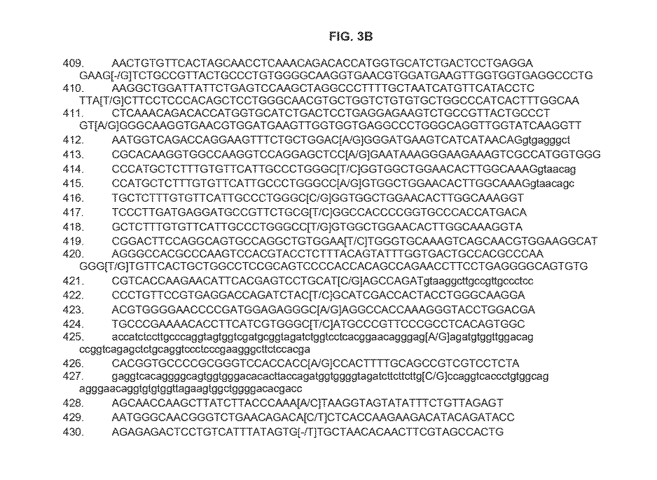

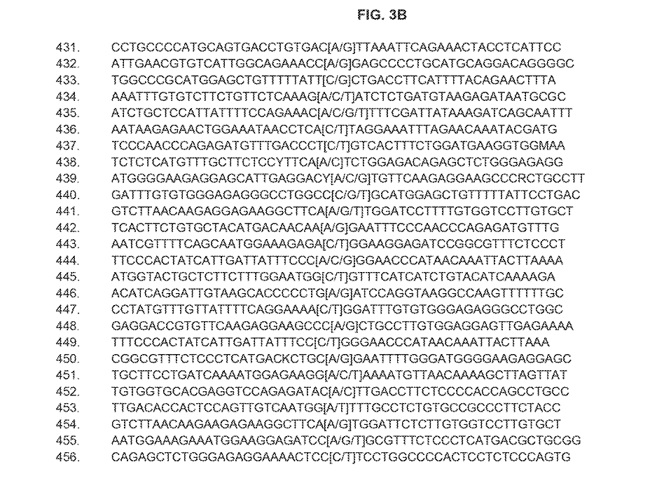

The SNVs of the invention are listed as part of SEQ ID NOS:1-511 (see FIGS. 3A and B) and are indicated in the larger nucleic acid sequence by a bracket containing a polymorphism, a mutation, a variant, an insertion of one or more nucleotides or deletion of one or more nucleotides. The bracket can, for example indicate that the variant is a toggle choice of nucleotides, for example “[A/T],” a deletion “[_]” and insertion “[X]” or [X . . . X].”

The SNVs of the invention are contained or embedded in larger nucleic acid sequences, referred to as “SNV-containing nucleic acid sequences.” These sequences are derived from the genes that comprise the SNV sequences. The SNV-containing nucleic acid sequences are at least 10 nucleotides in length and have over their length at least 70%, at least 75%, at least 80%, at least 85%, at least 90%, at least 95%, at least 96%, at least 97%, at least 98%, at least 99% or 100% contiguous sequence identity to a sequence selected from the group consisting of SEQ ID NO:1-511.

The SNV-containing nucleic acids are at least 10 nucleotides in length, 15, 20, 25, 30, 35, 40, 45, 50, 55, 60, 65, 70, 75, 80, 85, 90, 95, 100 or greater in length. As part of a microarray, the SNV-containing nucleic acids may have a uniform length but do not necessarily have a uniform length.

“Disease state,” “disease,” and “pathological condition” are used interchangeably herein, and refer to acquired or genetic diseases or a combination of both in a person, and also refer to a drug response to a disease. The terms also refer to a risk of a person acquiring or being diagnosed with such a disease.

“Cohort,” “ethnic group,” “national origin,” “demographic region” or “demographic segment” refer to a sub-population of persons or individuals. In one embodiment, these terms refer to a group of individual categorized by gender, age, weight, etc. In one embodiment, these terms refer to persons who are residents of or living in or born in or having relatives or ancestors in a continent, sub-continent, country, state, or region. In another embodiment, these terms refer to an ethnic group or individuals having common national origin. The sub-population of persons or individual described herein share certain common genetic backgrounds (including gender) and/or certain common environmental influences such as socio-economic status, diet, exposure to weather/elements and toxins, sedentary or active lifestyles, urban, suburban or rural lifestyles, leisure activities, family size, etc.

Genetic variant refers to the co-existence of two or more discontinuous forms of a genetic sequence. A “single nucleotide variant” or SNV, one of the most common genetic variants, is a small variation occurring within a single nucleotide in a deoxyribonucleic acid (DNA) sequence or other shared sequence. SNVs often occur at or near a gene found to be associated with a certain disease. Therefore, they are often good genetic markers indicative of how humans develop the disease and respond to drugs, chemicals and other agents, and how susceptible or resistant humans are to the disease. In the context of the present application SNV also refers to insertions, deletions, and polymorphims of one of more nucleotides, usually from about 1-10 nucleotides in length. An SNV can also include a rare point mutation in an individual, or a more common choice between two nucleotides at a specific position.

Physiologic, genomic, cellular, cohort, demographic data and the like are genetic, non-genetic, epigenetic, biochemical, micro or macrobiological, physiological, lifestyle etc., data that can be assessed along with the status of an SNV and may include, for instance, information about the DNA methylation, genetic copy number, micro RNAs, transcriptome, microbiome, proteome, epigenome, pathology data, histological data, biochemical data, personal data, clinical data or any combination thereof. Examples of such data include patient medical history (e.g., prior history of disease or symptoms), patient habits (e.g., smoking status, exercise habits, etc.), family history data, drug therapy data, radiological images (e.g., computed tomography (CT) images, X-ray images, etc.), radiological reports, doctor progress notes, details about medical procedures and/or examinations (e.g., time between first examination and follow-up), demographic information (e.g., age, race, gender, location, etc.), clinic measurement data (e.g., heart-rate, systolic and diastolic blood pressures, mean arterial blood pressure, etc.), laboratory test results, and so forth. Laboratory test results may include measurements of at least one bio-marker found in a biological sample (e.g., urine, blood, hair, etc.) taken from the patient including, for example, glucose, serum insulin, statin, albumin protein, high-density lipoprotein (HDL) cholesterol, low-density lipoprotein (LDL) cholesterol, brain natriuretic peptide (BNP), N-terminal pro b-type natriuretic peptide (NT-proBNP), glycosylated hemoglobin, testosterone, or any other quantifiable characteristic.

In addition, the non-genetic data may further include analytical data derived from the clinical data. For instance, analysis may be performed on the clinical data to generate parameters of clinical significance, such as body mass index (BMI), mean arterial pressure, pulse pressure (PP), patient lifestyle data (e.g., stress level), or other biochemical parameters.

“Biological sample” refers to a sample from a patient or individual containing nucleic acid to be analyzed by the methods of the invention. Samples include blood, saliva, skin cells, hair, urine, stool, tissue biopsies, and the like.

“Assess or assessment or health assessment” and “diagnosis” refer to a process of identifying a disease state in a person, which includes the use of tools such as symptoms, family history, test results and genetic markers to identify the presence of a disease state, or a predisposition to or possibility of acquiring the disease state.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to the full length of the reference sequence, usually about 25 to 100, or 50 to about 150, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher et al., J. Biol. Chem. 273(52):35095-35101 (1998).

“Cancer” refers to human cancers and carcinomas, sarcomas, adenocarcinomas, etc., including solid tumors, kidney, breast, lung, kidney, bladder, urinary tract, urethra, penis, vulva, vagina, cervical, colon, ovarian, prostate, pancreas, stomach, brain, head and neck, skin, uterine, testicular, esophagus, and liver cancer.

### DETAILED DESCRIPTION OF THE INVENTION

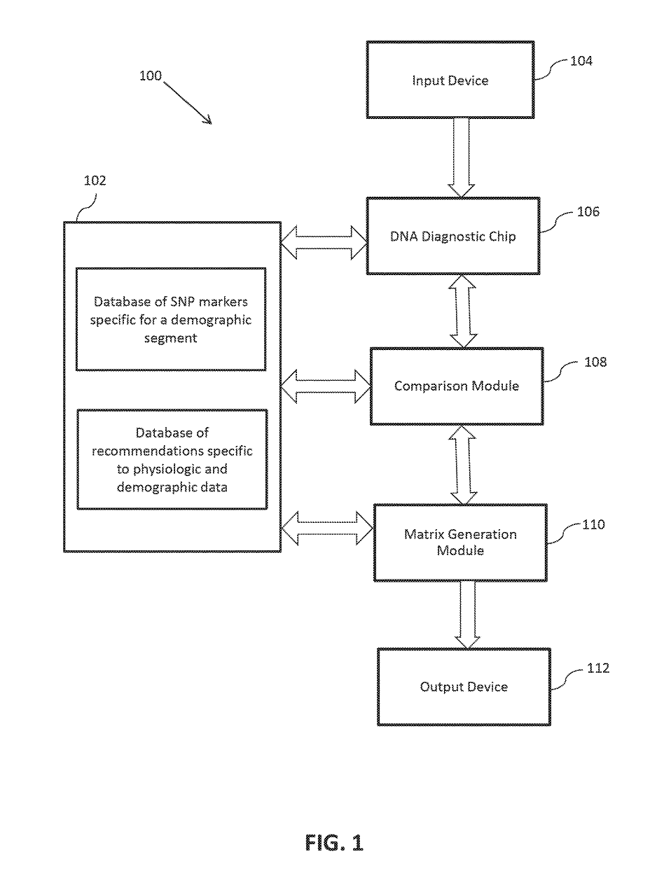

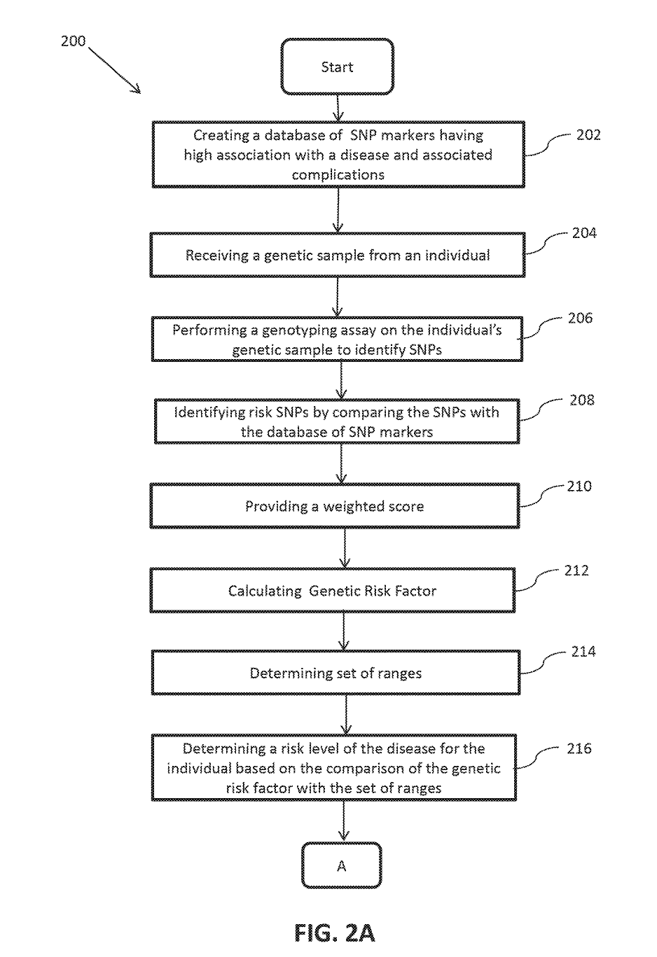

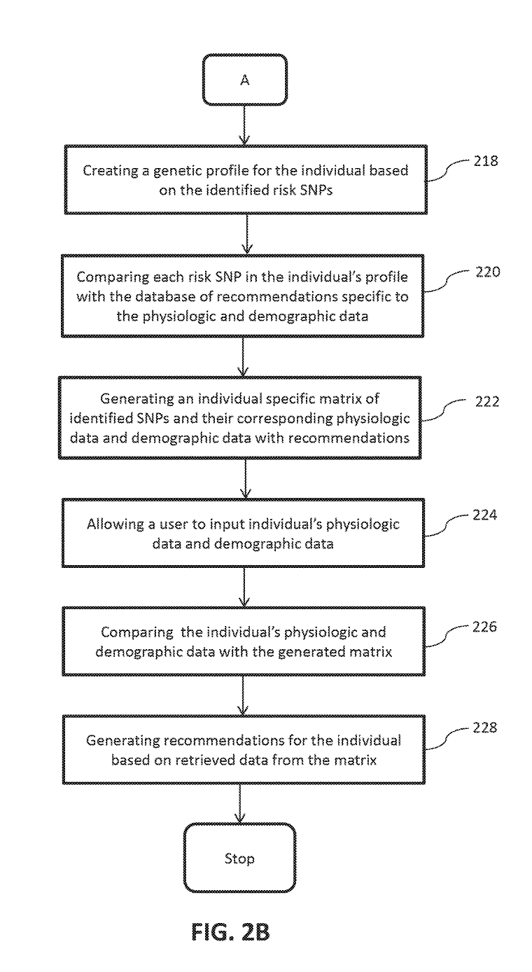

The present invention includes methods of disease diagnosis using SNVs specific to a demographic segment. The system 100 described in FIG. 1 and the method 200 described in the flow chart of FIG. 2 can be used for diagnosis and treatment of multiple diseases using SNV markers specific to a particular demographic segment.

The present invention also includes methods of disease diagnosis using SNVs specific to a demographic segment, which SNVs are immobilized as an array on a solid support (a “chip”), and kits for performing the diagnostic assays of the invention that comprise the arrays of the invention. The invention also includes primer sets for isolating and amplifying nucleic acid (DNA or RNA) including the SNVs from the subjects, and kits comprising the same. The appropriate primer set may be easily designed by those skilled in the art with reference to the SNV sequences according to an embodiment of the present invention.

The SNVs of the invention can, in one embodiment, be analyzed using genomic sequencing and computer databases comprising SNV sequences, or by hybridizing genomic samples to nucleic acid arrays immobilized on a solid support, or by amplification using PCR primers.

The system and method described herein can be used, for example, in the diagnosis and treatment of cancer, diseases of the eye, cardiometabolic diseases, inherited diseases, including pediatric diseases, and for pharmacogenetics.

In one embodiment the cancer is is breast cancer, ovarian cancer, and colon cancer. In another embodiment, the disease of the eye is glaucoma or age-related macular degeneration (AMID). In another embodiment, the method is used in pharmacogenetic methods to determine the best drug for disease treatment based on genetic profile, such as response to statins and warfarin.

In another embodiment, the cardiometabolic disease is an arrhythmia, e.g., long QT syndrome; clotting factor disorders, including drug response to warfarin; cardiomyopathy; coronary artery disease; cardiovascular disease, optionally associated with diabetes types I or II; hypertension; obesity; lipid disorders, such as high cholesterol, LDL, or triglycerides, or low levels of HDL, including drug response to statins; diabetes types I and II; maturity onset diabetes of the young (MODY); diabetes-associated retinopathy, obesity, enhanced waist circumference, and other complications such as neuropathy, nephropathy, foot damage, cardiovascular disease and stroke.

In another embodiment, the inherited disease (including pediatric diseases) is cystic fibrosis, congenital obstruction of the vas deferens, phenylketonuria, dopa response dystonia, epilepsy, homocystinuria, tyrosinemia, sickle cell anemia, thalassemia, Wilson's disease, non-ketotic hyperglycinemia (NKHG), glucose 6-phosphate dehydrogenase (G6PD) deficiency; maple syrup urine disease (MSUD); congenital adrenal hyperplasia.

The SNV-containing nucleic acids of the invention, or sequences thereof in a database, may be combined in any desired group. For example, SNV-containing nucleic acids may be grouped or combined in a disease specific database, microarray or PCR assay. For example, a database or chip may contain only those SNVs that are related to a particular disease state or pharmacogenetic application, such as cancer, diseases of the eye, cardiometabolic diseases, or inherited diseases, including pediatric diseases, as described herein. The database, microarray or PCR kit database can comprise at least 10, 20, 30, 40, 50, 60, 70, 80 or 90 SNV-containing nucleic acid sequences or at least 100, 200, 300, 400 or 500 SNV-containing nucleic acid sequences. Any combination and number of SNVs may be selected.

The nucleic acid microarrays and PCR primer kits and assays of the invention are used to identify sample nucleic acids containing an SNV of the invention, for diagnosis of or determination of a risk of a pathological condition. Microarray assays and PCR assays are conducted using hybridization and/or polymerization reactions with sample genetic material from the subject to be diagnosed. Hybridization assays are well known to those of skill in the art. For PCR assays, primers are designed to amplify regions of SNV-containing nucleic acids from the genetic sample. The resulting amplified nucleic acids can be identified with a microarray, or the microarray can be used to directly identify the SNV-containing nucleic acids in the genetic material. Suitable labels or sequencing techniques are used in the assays of the invention to identify SNVs of the invention and therefore providing a diagnosis or risk or developing a pathological condition.

The term “nucleic acid array” or sometimes “microarray” as used herein, refers to an intentionally created collection of nucleic acids which can be prepared either synthetically or biosynthetically and screened for hybridization to a sample sequence in a variety of different formats (for example, libraries of soluble molecules; and libraries of oligos tethered to resin beads, silica chips, or other solid supports). Additionally, the term “array” is meant to include those libraries of nucleic acids which can be prepared by spotting nucleic acids of essentially any length (for example, from 1 to about 1000 nucleotide monomers in length) onto a substrate.

The term “primer” as used herein, refers to a single-stranded oligonucleotide capable of acting as a point of initiation for template-directed DNA synthesis (PCR) under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 10-15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The phrase “stringent hybridization conditions” refers to conditions under which a probe or primer will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., John Wiley & Sons.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

The present invention includes kits comprising components useful to practice a method or use disclosed herein. In one embodiment, a kit comprises a microarray of SNVs of the invention or primers for PCR amplification of SNVs of the invention, optionally a means for obtaining a nucleic acid sample from a subject, and optionally one or more carriers, one or more adjuvants and one or more pharmaceutical components. Such kits can be used to practice diagnostic methods described herein. Kits can be portable, for example, and used in the home, or able to be transported to the field. Other kits may be of use in a health facility to analyze a subject suspected of or being testing for a risk of having a pathological condition.

Kits can also include a suitable container, for example, a vessel, vials, tubes, mini- or microfuge tubes, test tube, flask, bottle, syringe or other container. Where an additional component or agent is provided, the kit can contain one or more additional containers into which this agent or component may be placed. Kits herein will also typically include a means for containing the agent (e.g. a vessel), composition and any other reagent containers in close confinement for commercial sale. Such containers may include injection or blow-molded plastic containers into which the desired vials are retained.

A kit disclosed herein may include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk (e.g., hard disk, flash memory), optical disk such as CD- or DVD-ROM/RAM, DVD, MP3, magnetic tape, or an electrical storage media such as RAM and ROM or hybrids of these such as magnetic/optical storage media, FLASH media or memory type cards. Labels or inserts may include identifying information of one or more components therein, and assay conditions. Labels or inserts can include information identifying manufacturer information, lot numbers, manufacturer location and date.

Labels or inserts can include information on a condition, disorder or disease diagnosis for which a kit component may be used. Labels or inserts can include instructions for the clinician or subject for using one or more of the kit components in a method of diagnosis.

Those with ordinary skill in the art will appreciate that the elements in the figures are illustrated for simplicity and clarity and are not necessarily drawn to scale. For example, the dimensions of some of the elements in the figures may be exaggerated, relative to other elements, in order to improve the understanding of the present invention.

There may be additional components described in the foregoing application that are not depicted on one of the described drawings. In the event such a component is described, but not depicted in a drawing, the absence of such a drawing should not be considered as an omission of such design from the specification.

The present invention can utilize a combination of system components, which constitute a system and method for diagnosing disease and generating treatment and/or recommendations using single nucleotide variant (SNV) markers, in accordance with an embodiment of the present invention. Accordingly, the components and method steps have been represented, showing only specific details that are pertinent for an understanding of the present invention so as not to obscure the disclosure with details that will be readily apparent to those with ordinary skill in the art, having the benefit of the description herein.

As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, and as a representative basis for teaching one skilled in the art to variously employ the present invention in virtually any appropriately detailed structure. Further, the terms and phrases used herein are not intended to be limiting, but rather to provide an understandable description of the invention.

FIGS. 1 and 2

The invention provides a system 100 and a method 200 for the analysis of each single nucleotide variant (SNV) genotyping assay in a person for diagnosis of a pathological condition in a person and, in one embodiment, for providing a preventive health care recommendation to the person based on the diagnosis, and/or the calculation of a genetic risk factor for the pathological condition of the person. The preventive or wellness recommendations are based on an individual's genetic, demographic and physiologic data. The wellness recommendation can be, but is not limited to, exercise, diet, lifestyle modifications, drugs, and the like. The pathological condition may involve a disease, susceptibility for a disease; a reaction to drugs, congenital disorders etc.

The block diagram of system 100 for diagnosing disease and generating recommendations using SNV markers, in accordance with an embodiment of the present invention, is shown in FIG. 1. The system 100 includes a database 102, an input device 104, a DNA diagnostic chip 106, a comparison module 108, a matrix generation module 110, and an output device 112.

The database 102 includes a database of SNV markers (FIG. 3) specific to a demographic segment and database of recommendations specific to physiologic and demographic data. The SNV markers are related to pathological disorders and recommendations are collated from various international guidelines. The international guidelines may be according to, but are not limited to, the Indian Medical Association or the American Cancer Society. The input device 104 is used to provide a genetic sample of an individual who needs to be tested for the disease.

The DNA diagnostic chip 106 receives the genetic sample and performs a genotyping assay on the genetic sample to identify a plurality of SNVs. The DNA diagnostic chip 106 further compares the plurality of SNVs with the database of SNV markers 102 to identify a plurality of risk SNVs, which are associated with the pathologic conditions. The DNA diagnostic chip 106 generates a computer-readable format corresponding to the plurality of risk SNVs. In an embodiment, the computer readable format is the genetic risk factor (GRF) calculated by the DNA diagnostic chip 106.

The GRF calculated is then compared with a set of ranges by the comparison module 108. The set of ranges is decided based on the number of SNV markers. Depending on the comparison of the GRF coming under one of the set of ranges, the comparison module 108 identifies the pathological condition and stores it in the database 102. Based on the identified pathological condition, demographic and physiologic conditions, and corresponding recommendations, a matrix is generated using the matrix generation module 110. The input device 104 allows a user to enter individual specific physiologic data and demographic data. Further, the physiologic data is compared with the matrix generated, to generate healthcare-related recommendations to treat and prevent the listed physiologic problems and pathological disorder. The healthcare-related recommendations are displayed using the output device 112, which generates a computer readable format corresponding to the recommendations generated.

In an embodiment, the comparison module 108 may be a computer, microcontroller, processor, and the like. In another embodiment, the matrix generating module 110 may be a processor, microcontroller, computer, analyzer, and the like. In yet another embodiment, the database 102 may be any storage device, RAM, memory card, hard drive, external hard disk, and the like. In yet another embodiment, the output device may be any display device, computer, tablet, monitor, printer, and the like.

The method 200 for diagnosing disease and generating recommendations using SNV markers, according to an embodiment of the disclosure, is shown in the flowchart of FIG. 2. The method involves at first step 202 creation of a panel of single nucleotide variant (SNV) markers that have a high association with the disease and associated complications, and are specific to a demographic segment to which the person belongs. The panel of SNV markers is a form of database of SNV markers. In an embodiment of the present invention, the SNV markers are specific to the Indian population (FIG. 3). The panels of SNV markers of FIG. 3 are representative of diseases that are common in India. The database is prepared through an extensive review of public databases and medical literature to identify demography-specific genetic variants in genes associated with the disease.

Further, step 202 also involves creation of a database of healthcare-related recommendations specific to physiological and demographic data. The physiologic data includes, but is not limited to, blood pressure, weight, height, obesity risk, and the like. In an embodiment of the present invention, the database for healthcare-related recommendation is created based on clinical criteria and guidelines as per the Indian medical association. Some of the other public databases and medical literature include, but are not limited to, guidelines by the American Cancer Society, the Drug Bank, the Therapeutic Target DB, STITCH, Supertarget, PubMed, AMEDEO, and the like.

The next step 204 includes receiving a genetic sample of an individual. The genetic sample can be retrieved from the individual using any available methods such as, but not limited to, extraction from any tissue, commonly blood, saliva or buccal swab, and the like. Further at step 206, a genetic sample of the individual is assayed using genotyping assay. This results in identification of a plurality of SNVs in the genetic sample of the individual. The genotyping assay is performed individually for each genetic sample using available methods such as, but not limited to, Microarray Genotyping, Molecular Beacon Genotyping, 50 Nuclease Assay, Invader Assay, and the like.

At next step 208, the pluralities of SNVs are compared with the database of SNV markers. These results in identification of a plurality of risk SNVs, which are associated with diseases and their associated complications. Normally, the disease is associated with a plurality of risk genes or risk SNVs. The presence of these risk genes or risk SNVs determines the possibility of the individual having the disease.

In an embodiment, comparison of each SNV is binary, i.e., either yes or no. If yes, the SNV is a carrier for the pathological condition. If no, the SNV is not a carrier for the pathological condition. The results may be yes for some SNVs and no for other SNVs. Normally, each of the plurality of risk genes has at least two copies in a genome of the individual. If both the copies of the risk gene are present in the genome, the risk gene is referred to by the symbol +/+, which indicates the highest risk of having the disease in relation to that particular SNV. If one copy of the risk gene is present in the genome, the risk gene is referred to by the symbol +/−, which indicates a medium possibility of having the disease. And, if any of the copies of the risk gene is not present in the genome, the risk gene is referred to by the symbol −/−, which indicates the least possibility of having the disease. The terms disease and disease are used interchangeably henceforth.

At the next step 210, each of the plurality of risk SNVs is given a weighted score for the three possibilities discussed above, i.e., for +/+, +/− and −/− based on an odds ratio. For calculating the odds ratio, two population sets are observed. The first population set is exposed to conditions that favor development of a disease and the second population set is used as a control. The odds ratio is the odds of a person having the disease when exposed (in numerator) to the odds of the person having the disease when not exposed (in denominator). In the numerator, the odds of a person having the disease when exposed is calculated using the number of individuals having the disease who were exposed (A1) divided by the number of individuals not having the disease who were exposed (A2). In the denominator, the odds of the person having the disease when not exposed is calculated using the number of individuals having the disease who were not exposed (A3) divided by the number of individuals not having the disease who were not exposed (A4). This can be shown in the equation [1] below:

Odds ratio=(A1/A2)/(A3/A4)

The weighted score is given, based on the odds ratio. If the odds ratio is high, a high weighted score is given to the particular SNV. This method provides a weighted addition to the risk of having the disease. Since the method is using additive method, the method down regulates the overall effect. This method also allows determination of which SNV out of a plurality of risk SNVs has the maximum effect in causing the disease.

At step 212, a genetic risk factor (GRF) is calculated. The GRF is the addition of the weighted scores of all the SNVs divided by twice the number of SNVs, since there are two copies of each of the plurality of risk genes in the genome of the person, as shown in the formula of equation [2]:

GRF=(addition of weighted score of all the SNVs)/(No. of SNVs*2)

However, it should be appreciated that calculation of GRF is not limited to the formula mentioned in equation [2] and many other variations can be used to calculate the GRF.

In the next step at 214, a plurality of set of ranges are determined based on the database of SNV markers and the set of ranges representing a plurality of risk levels of the disease for the individual. In an embodiment of the present invention, the set of ranges are distinctly separated and defined into three parts, based on the total number of SNVs associated with the disease. If the GRF is less than one-third of the total number of SNVs, which constitute the genome, the person is at a low risk of getting the disease. If the GRF is between one-third and two-third of the total number of SNVs, the person is at a medium risk of getting the disease. If the GRF is greater than two-third of the total number of SNVs, the person is at a high risk of getting the disease. And at step 216, the GRF value calculated at the previous step 212 is compared with the set of ranges to determine a risk level of the set of risk levels. Based on the GRF value, the risk level of the disease identified is determined.

Further, next step 218, involves creation of a genetic profile of the individual in the database, based on the risk level of the identified disease calculated at previous step 216. Next step 220 involves comparison of each risk SNV in the individual's genetic profile with the database of recommendations specific to physiologic data and demographic data. In an embodiment, the physiologic conditions may be, but are not limited to, high/low blood pressure, weight, height, obesity risk, and the like.

At step 222, a matrix is generated with complete coverage of risk SNVs the disease, physiologic and demographic data, and their corresponding recommendations. The matrix generated is individual-specific and provides information on healthcare-related recommendations to prevent the disease and physiologic disorders. In an embodiment, the recommendations depend on the risk level calculated for the disease. In another embodiment, the recommendations can be pharmaceutical and non-pharmaceutical, where non-pharmaceutical recommendations can be exercise, diet, lifestyle modifications, and the like. Further, in step 224, a user enters personalized physiologic data into the database. At step 226, the entered physiologic data from step 222 is compared with the matrix generated at step 222. Based on the compared data, recommendations are generated in step 228. In an embodiment, the recommendations can also include other healthcare-related information, such as drug dosage, the efficiency of a specified drug, necessary healthcare precautions, and the like. In another embodiment, the user is allowed to select any set of physiologic factors from all available physiologic factors. If the available physiological factors include heart health, obesity risk, or predisposition to metabolic syndrome, the user can select one or more physiological factors to input the data. In an example, the individual can have heart assessment on a particular day, his or her metabolic profile the next day and drug profile on yet another day. The individual may input the desired physiologic factors and the corresponding recommendations are generated accordingly. Thus, the user is provided the option of controllability.

Preventive healthcare-related recommendations vary from disease to disease. In an embodiment, these recommendations may include a detailed treatment plan, a diet plan for the individual, recommended medicines, therapies or tests (including screening and diagnostic tests) required, and the like.

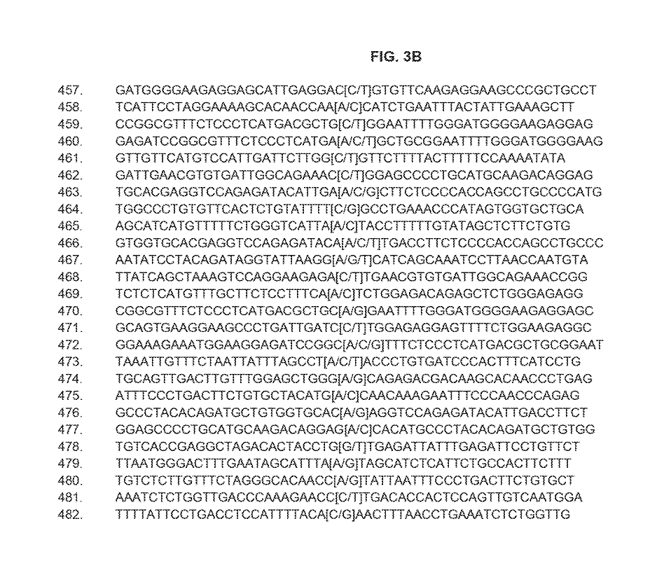

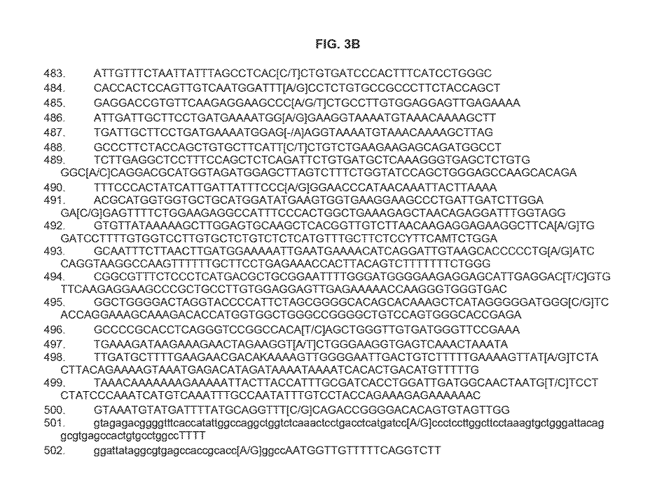

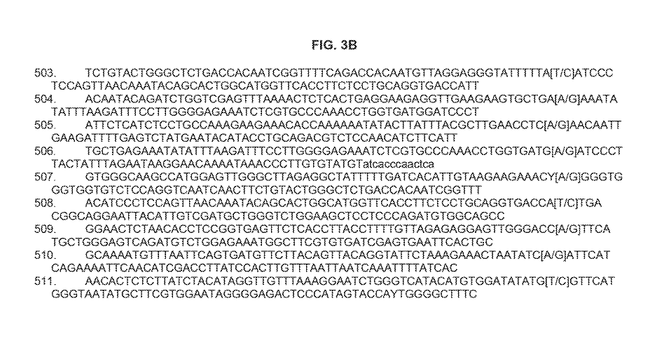

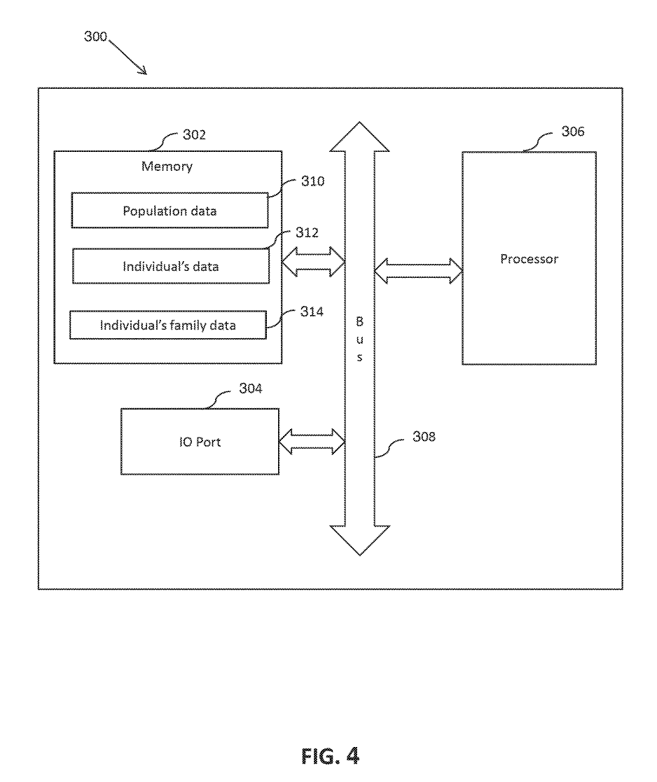

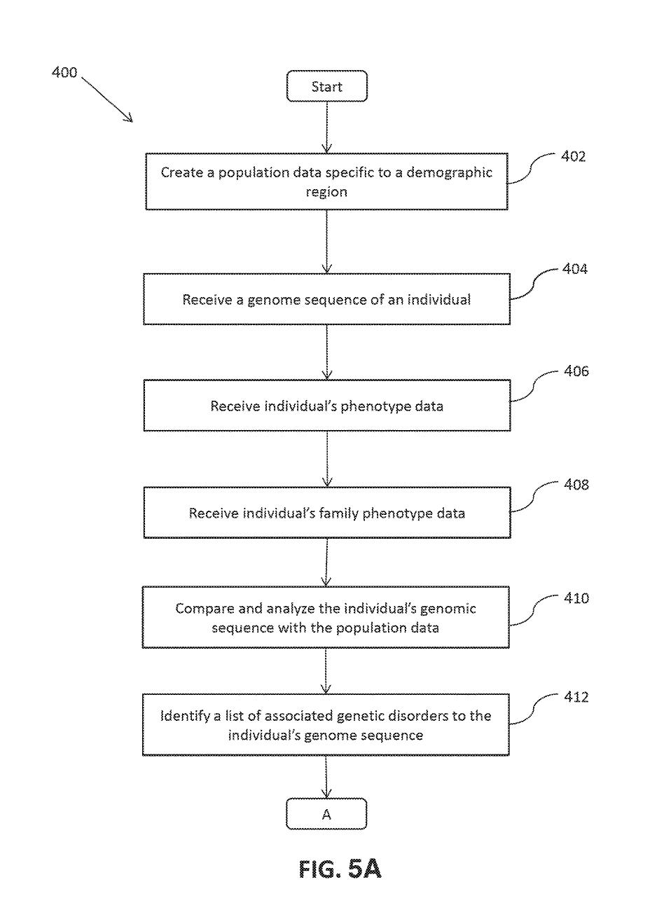

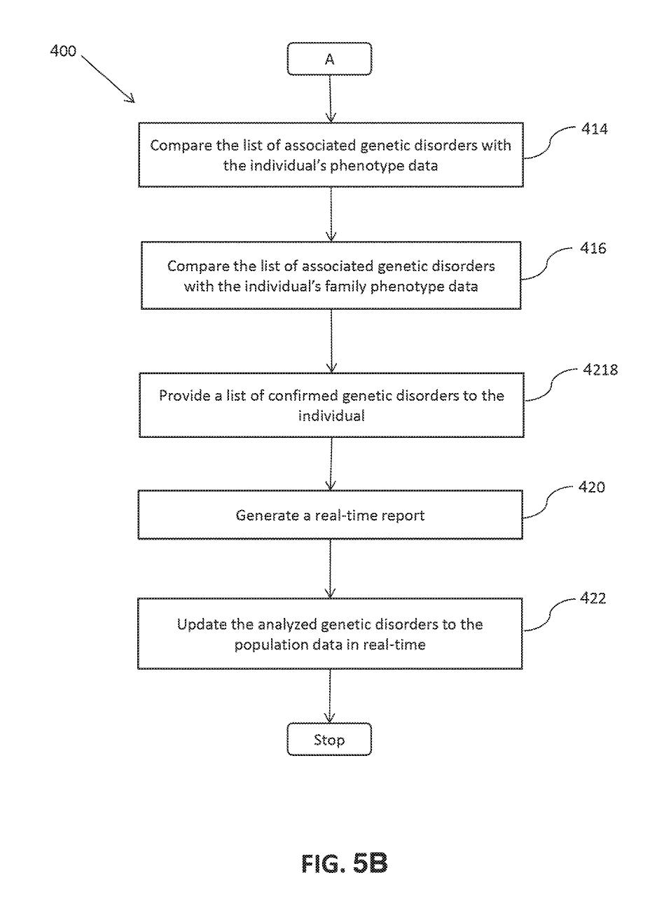

FIGS. 4 and 5

FIG. 4 shows the computer system 300 which includes instructions that are required to perform the methodologies described herein. The computer system 300 may be implemented as a server machine or a client machine in a client-server computer network or as a peer machine in a peer-to peer or distributed network. The computer system 300 may be a personal computer, a laptop, a server, a set-top box (STB), a tablet, a PDA, a cellular telephone, a web appliance, a server, a network router, a network switch, a network bridge, or any machine capable of executing a set of computer instructions (sequential or otherwise). In an embodiment, at least one of the components of the computer system 300 may be incorporated into other systems such as a genome machine, Laboratory Information Management Systems and Electronic Medical Record Systems.

Further, while only a single computer system 300 is illustrated in FIG. 4, the term ‘computer system’ should also be taken to include any collection of systems that individually or jointly execute a set (or multiple sets) of instructions to perform any one or more of the methodologies discussed herein.

The computer system 300 of FIG. 4 includes a memory 302, an input/output (TO) port 304, a processor 106, and a system bus 308. The memory 302 stores sets of instructions to perform various functions described herein. The IO port 304 is an interface between the computer system 300 and an external network such as the Internet. The IO port 304 may be connected to input devices such as keyboards, touch-sensitive input devices, microphones, and so on, to accept input from the user. Further, the IO port 304 may be connected to output devices such as desktops, printers, laptops, cellular telephones, and tablets. The memory 302 and the IO port 304 communicate by way of the system bus 308. The processor 306 fetches and executes the sets of instructions from the memory 302. The computer network may include wired and wireless networks such as the Internet, Local Area Networks (LANs), Metropolitan Area Networks (MANs), mobile networks, and the like. In an embodiment of this specification, the computer network is the Internet.

The memory 302 stores population data 310, which is fed to the memory 302 from external databases such as public databases, medical and clinical reports, scientific and medical journals, and online databases. The population data 310 is specific to a demographic region, which may be but is not limited to countries such as India, the USA, UK and Singapore, and states such as Tamil Nadu, Maharashtra and Kerala. The population data 310 includes genome sequences with their corresponding gene variants. These gene variants are associated with phenotype data. For an example, the population data 310 has a genome code for extra finger gene mutation and its corresponding phenotype data includes extra finger. In an embodiment, information relating to the population data 310 may be specific, but not limited to race, gender, and age.

An input device connected to the IO device 304 enables a user to enter individual's data 312, which includes genomic and phenotype data relating to the individual. The genomic data constitutes the genome sequence of the individual and the phenotype data is clinical data of the individual. Further, the input device allows the user to enter the individual's family data 314, which includes the phenotype data of the individual's family. The phenotype data constitutes clinical data relating to the individual's family. In an embodiment, the individual's data 312 and the individual's family data 314 are entered manually by, but not limited to, the individual, the individual's family, a physician or a chemist. In another embodiment, the user may upload the individual's data 312 and the individual's family data 314 as one of the medical, clinical and genetic reports. Further, at least one of the entered, uploaded, and received genotype and phenotype data is transferred to the memory 302 through the IO port 304.

The processor 306 fetches the individual's genome sequence from the memory 302. The fetched genome sequence is analyzed and conceptualized as a cartridge against the population data 310. This results in identification of a plurality of gene variants in the individual's genome sequence. Each of the plurality of gene variants results in a genetic disorder with the corresponding phenotypic data. The identified plurality of gene variants is overlaid on the individual's phenotype data. This helps to check the extent of relevancy between the determined gene variants and the individual's phenotype data. For an example, the analyzed genomic sequence of the individual has five gene variants that result in disorders. When the five gene variants are overlaid on the individual's phenotype data, it is identified that only two of the five gene variants have observable traits, as given in the individual's phenotype data.

Further, the processor 306 fetches the individual's family data 314 from the memory 302 and aligns with the individual's phenotype data. The identified plurality of gene variants is overlaid on the individual's fetched phenotype data and the individual's family data 314. This analysis enhances the extent of relevancy between the determined gene variants and the individual's phenotype data and the individual's family data 314. For example, the analyzed genomic sequence of the individual has five gene variants, which results in disorders. When the five gene variants are overlaid on the individual's phenotype data and the individual's family data 314, it is identified that four of the five gene variants have observable traits, as given in the individual's phenotype data and the individual's family data 314. The resulting overlay between the individual's phenotype data and family data and the determined gene variants will enable a “current understanding” output for the individual and his or her physician.

In each case, the analysis will be repeated, and if there is a change in output, a “revised real time” report will be generated, unless the individual specifies that there are to be no updates or limits their number/frequency. The individual's phenotype data and individual's family data 314 are meshed with previous analysis to match them. The modified analysis is updated to the population data 310. The update of the population data 310 happens in real time. In an embodiment, the genomic analyzer provides the user with options “yes” or “no” to determine whether the individual's family members are genotyped or not. If the answer is “yes,” the user enters the genotype of his or her family members. This is then meshed with the genomic sequence of the individual. If the answer is “no,” the genomic analyser continues with the next step.

FIGS. 5A and B illustrate a flowchart with the steps involved in a method 400 for analyzing and relating genomic data with phenotype data, in accordance with an embodiment of the present invention.

At step 402, the database of the population data 310 is stored in the memory 302. Further, at steps 404 and 406, the individual's data 312 is received from the input devices connected through IO port 304 and is stored in the memory 302. At step 408, the individual's family phenotype data is received from the input devices connected through the IO device 404 and stored in the memory 402.

At step 410, the processor 306 compares and analyzes the genome sequence of the individual's data 312 with the population data 310 to identify a list of associated genetic disorders at step 412. Further, at step 414, the processor 306 compares the list of associated genetic disorders with the individual's phenotype data to determine genetic disorders with observable traits. If at least one of the individual's specific genetic disorders remains unmapped, at step 416, the processor 306 compares and analyzes his or her genetic disorders without observable traits with his or her family phenotype data 314, and correspondingly updates the list of individual-specific genetic disorders to generate a validated individual-specific genotype-phenotype data at step 418.

In an embodiment, if at least one of the individual's family members is genotyped, the processor 306 meshes the genotype with the individual's genomic sequence. At step 420, the validated individual-specific genotype-phenotype data is transferred to the output devices connected through IO device 304 in order to generate an updated personalized report. Further, at step 422, the processor 306 updates the population data 310 stored in the memory 302 with the validated individual-specific genotype-phenotype data. A new population data 310 is then generated such that the updated associated genotype-phenotype data replaces the unmapped genotype-phenotype data in real time. The real time update enables a new user to extract the most relevant and accurate genotype-phenotype data. The method 400 uses data from different domains and meshes them across different dimensions to improve an interpretation of the individual's genomic data.

### ASPECTS OF THE INVENTION

In a first aspect, the invention provides a method for assessing or diagnosing a pathological condition in a person, the method comprising:
receiving a genetic sample of the person;
accessing a nucleic acid sequence database, the database comprising one or more single nucleotide variant (SNV)-containing nucleic acid sequences, wherein each SNV has an association with the pathological condition in a demographic segment to which the person belongs; wherein the SNV is selected from the group consisting of the SNV of SEQ ID NOS:1-511; and wherein the SNV-containing nucleic acid sequence is at least 10 nucleotides in length and has over its length at least 70%, at least 75%, at least 80%, at least 85%, at least 90%, at least 95%, at least 96%, at least 97%, at least 98%, at least 99% or 100% contiguous sequence identity to a sequence selected from the group consisting of SEQ ID NO:1-511;
performing a genotyping assay on the genetic sample to identify an SNV marker in the genetic sample of the person;
identifying a risk SNV in the genetic sample of the person by comparing the SNVs in the genetic sample of the person with the database of SNV markers; and
providing a diagnosis of a pathological condition in the person based on the identification of risk SNVs in the genetic sample of the person.

In another aspect, the invention provides a microarray of nucleic acids, the microarray comprising one or more single nucleotide variant (SNV)-containing nucleic acid sequences; wherein the SNV is selected from the group consisting of the SNV of SEQ ID NOS:1-511; and wherein the SNV-containing nucleic acid sequences are at least 10 nucleotides in length and have over their length at least 70%, at least 75%, at least 80%, at least 85%, at least 90%, at least 95%, at least 96%, at least 97%, at least 98%, at least 99% or 100% contiguous sequence identity to a sequence selected from the group consisting of SEQ ID NO:1-511.

In one embodiment, the database or microarray comprises at least 10, 20, 30, 40, 50, 60, 70, 80 or 90 SNV-containing nucleic acid sequences. In another embodiment, the database or microarray comprises at least 100, 200, 300, 400 or 500 SNV-containing nucleic acid sequences. In another embodiment, the database comprises 511 SNV-containing nucleic acid sequences.

In one embodiment, the SNV-containing nucleic acid sequences are 15, 20, 25, 30, 35, 40, 45, 50, 55, 60, 65, 70, 75, 80, 85, 90, 95, or 100 nucleotides or greater in length.

In one embodiment, the data base or microarray consists of SNV-containing nucleic acids associated with a pathological condition is selected from the group consisting of cancer, diseases of the eye, cardiometabolic diseases, inherited diseases, pediatric diseases, and pharmacogenetic responses to pathological conditions. In another embodiment, the pathological condition is selected from the group consisting of cancer, diseases of the eye, cardiometabolic diseases, inherited diseases, pediatric diseases, and pharmacogenetic responses to pathological conditions.

In one embodiment, the cancer is selected from the group consisting of breast cancer, ovarian cancer, and colon cancer, in another embodiment,

the disease of the eye is glaucoma or age-related macular degeneration (AMD). In another embodiment, the cardiometabolic disease is selected from the group consisting of arrhythmia, e.g., long QT syndrome; clotting factor disorders, including drug response to warfarin; cardiomyopathy; coronary artery disease; cardiovascular disease, optionally associated with diabetes types I or II; hypertension; obesity; lipid disorders, such as high cholesterol, LDL, or triglycerides, or low levels of HDL, including drug response to statins; diabetes types I and II; maturity onset diabetes of the young (MODY); diabetes-associated retinopathy, obesity, enhanced waist circumference, and other complications such as neuropathy, nephropathy, foot damage, cardiovascular disease and stroke. In another embodiment, the inherited disease or pediatric disease is selected from the group consisting of cystic fibrosis, congenital obstruction of the vas deferens, phenylketonuria, dopa response dystonia, epilepsy, homocystinuria, tyrosinemia, sickle cell anemia, thalassemia, Wilson's disease, non-ketotic hyperglycinemia (NKHG), glucose 6-phosphate dehydrogenase (G6PD) deficiency; maple syrup urine disease (MSUD); and congenital adrenal hyperplasia.

In one embodiment, the method comprises providing a preventive healthcare recommendation to the person.

In another embodiment, the demographic segment is residents of India.

In another aspect, the invention provides a microarray or a kit comprising PCR primers, the primers or microarray hybridizing to one or more single nucleotide variant (SNV)-containing nucleic acid; wherein the SNV is selected from the group consisting of the SNV of SEQ ID NOS:1-511; and wherein the SNV-containing nucleic acid sequences are at least 10 nucleotides in length and have over their length at least 70%, at least 75%, at least 80%, at least 85%, at least 90%, at least 95%, at least 96%, at least 97%, at least 98%, at least 99% or 100% contiguous sequence identity to a sequence selected from the group consisting of SEQ ID NO:1-511.

In another aspect, the invention provides a system for diagnosing a pathological condition in a person, the pathological condition having a risk SNV associated therewith, the risk SNV provided with a weighted score based on an odds ratio corresponding to each risk SNV, the system comprising:
an input device for receiving a genetic sample of the person;
a database comprising one or more single nucleotide variant (SNV)-containing nucleic acid sequences, wherein each SNV has an association with the pathological condition in a demographic segment to which the person belongs; wherein the SNV is selected from the group consisting of the SNV of SEQ ID NOS:1-511; and wherein the SNV-containing nucleic acid sequence is at least 10 nucleotides in length and has over its length at least 70%, at least 75%, at least 80%, at least 85%, at least 90%, at least 95%, at least 96%, at least 97%, at least 98%, at least 99% or 100% contiguous sequence identity to a sequence selected from the group consisting of SEQ ID NO:1-511
a DNA diagnostic chip in communication with the database of SNV markers, the DNA diagnostic chip configured to perform a genotyping assay on the genetic sample for identifying a plurality of SNVs, compare the plurality of SNVs with the database of SNVs for identifying a plurality of risk SNVs, and calculating a genetic risk factor for each risk SNV of the plurality of risk SNVs using the corresponding weighted score;
a comparison module for comparing the genetic risk factor and a plurality of set of ranges, the set of ranges representing the risk level of a disease on the person; and
an output device configured to provide a risk level of the set of risk levels for the person based on the comparison of the genetic risk factor with the set of ranges.

### EXAMPLES

### Example 1

The steps involved in the flowchart of FIG. 2 can also be explained using an example as follows. A certain disease “Type 2 diabetes mellitus” (DM) has three risk genes associated with it i.e. TCERG1L as SNV#101, TMEM163 as SNV#102 and TGFBR3 as SNV#103. Based on literature, each SNV is given a weighted score that is based on the corresponding odds ratio as follows:

| SNVs | Weighted Score |
|---|---|
| SNV #101 TCERG1L | +/+ = 4+ |
| (Odds ratio 1.8) | +/− = 2+ |
| | −/− = 0 |
| SNV#102 TMEM163 | +/+ = 3+ |
| (Odds ratio 1.6) | +/− = 1+ |
| | −/− = 0 |
| SNV#103 TGFBR3 | +/+ = 2+ |
| (Odds ratio 1.2) | +/− = 1+ |
| | −/− = 0 |

Assuming an individual (A) has following SNVs in his genomic profile 101+/+; 102+/+; 103+/− then the GRF value will be equal to (4+3+1)/(3*2)=1.3

And

Another individual (B) has following SNVs in his genome profile 101−/−; 102−/+; 103−/− then the GRF value will be equal to (0+1+0)/(3*2)=0.2

Now based on the determined set of ranges

If total is less than 0.4, the individual is at low risk

If total is between 0.4 and 0.7, the individual is at medium risk

If total is more than 0.7, the individual is at high risk

Therefore, the individual (A) is at high risk for developing type 2 diabetes mellitus and the individual (B) is at low risk for developing type 2 diabetes mellitus when compared to the general population.

### Example 2

The steps involved in the flowchart of FIG. 2 can also be explained using an example as follows.

A person is advised to undergo a genetic test, which involves extracting his or her DNA sample from saliva, blood, and the like, and analyzing the sample. Following the analysis, risk genes are identified which correspond to a genetic disorder “hypertension,” after comparing this with database guidelines. Hypertension has three risk genes associated with it, i.e., rs4149601 as SNV#101, rs2288774as SNV#102, and rs3865418as SNV#103. Based on literature, each SNV is given a weighted score that is based on the corresponding odds ratio as follows:

| SNVs | Weighted Score |
|---|---|
| SNV #101 rs4149601 | +/+ = 4+ |
| (Odds ratio 1.8) | +/− = 2+ |
| | −/− = 0 |
| SNV#102 rs2288774 | +/+ = 3+ |
| (Odds ratio 1.6) | +/− = 1+ |
| | −/− = 0 |
| SNV#103 rs3865418 | +/+ = 2+ |
| (Odds ratio 1.2) | +/− = 1+ |
| | −/− = 0 |

Assuming the person has the following SNVs in his or her genomic profile 101+/+; 102+/+; 103+/−, the GRF value will be equal to (4+3+1)/(3*2)=1.3

Now based on the determined set of ranges:

If the total is less than 0.4, the individual is at low risk.

If the total is between 0.4 and 0.7, the individual is at medium risk.

If the total is more than 0.7, the individual is at high risk.

Therefore, it is concluded that the person is at high risk of developing hypertension. Based on the aforementioned data, a genetic profile of the individual is created, specifying the 101+/+; 102+/+; 103+/−genes, GRF value of 1.3 and indicating high risk. Thereafter, based on the genetic profile, a matrix is generated, which includes physiologic and demographic data with corresponding recommendations pertaining to the individual's genetic profile. Let us assume that the individual, aged<30 years, with weight of 60 kgs and iron deficiency (physiologic data) is from Maharashtra (demographic data). The aforementioned data is entered in the system 100 by the user and compared with the matrix data to give corresponding recommendations, i.e., a low-sodium diet and a minimum 30-minute walk to prevent risk of hypertension.

In an embodiment, the matrix is updated regularly, based on a variation in healthcare-related recommendations and physiological conditions. The recommendations are categorized and generated, based on the individual's genetic profile. It should also be appreciated that the method 100 is not specific to demography-specific disorders and but can be used to generate recommendations, based on one or more of an individual's physiological conditions. The method 100 also provides flexibility to select specific physiological conditions desired by an individual, and therefore, provides an option of controllability. The method 100 can also be used for diagnosis and treatment of various other diseases, which are clinically actionable by a person skilled in the art.

### Example 3

The steps involved in the flowchart of FIG. 2 can also be explained by using the following example:

A certain disease “Thalassemia” has a risk gene associated with it, i.e., HBB as SNV#101.

Case I: Let us assume that an individual (A) has SNV#102, SNV#101 and SNV#104 in his or her genomic profile in the respective order and A's partner's data is not available in the database. At step 208, SNV#102 is compared with the database. At step 210, a risk SNV is not detected, and hence, the counter increments by one to compare the next SNV with the database. Since SNV#101 is a risk SNV, the process moves to the next step, i.e., step 212, and determines whether A's partner's data is present. Since A's partner's data is not available in the database, a first healthcare-related recommendation is generated, which includes advice to repeat method 200 for the individual's partner and/or perform sequencing of the entire gene of the individual's partner to detect rare mutations not picked up by method 200.

Case II: Let us assume that an individual (B) has SNV#101, SNV#102 and SNV#103 in his or her genomic profile in the respective order and B's partner has SNV#104, SNV#105 and SNV#106 in his or her genomic profile, and the data is available in the database. In such a scenario, a risk SNV is detected at step 210, since SNV#101 is a risk SNV. The process moves to the next step 212 and determines whether B's partner's data is present. Since B's partner's data is available in the database, the process moves to the next step of detecting the presence of SNV#101 in B's partner's data. Since SNV#101 is not present in B's partner's data, a second healthcare-related recommendation is generated, which includes advice on performing sequencing of the entire gene of the individual's partner to detect rare mutations not picked up by method 200.

Case III: Let us assume that an individual (C) has SNV#101, SNV#102 and SNV#103 in his or her genomic profile in the respective order and C's partner has SNV#101, SNV#105 and SNV#106 in his or her genomic profile, and the data is available in the database. In such a scenario, a risk SNV is detected at step 210, since SNV#101 is a risk SNV The process moves to the next step 212 and determines whether C's partner's data is present. Since C's partner's data is available in the database, the process moves to the next step of detecting the presence of SNV#101 in C's partner's data. Since SNV#101 is present in C's partner data, a third healthcare-related recommendation is generated, which provides information pertaining to the individual's estimated susceptibility to have offspring with the inherited genetic disorder as 25%. The individual is advised to seek genetic counseling.

The method 200 described in the flowchart of FIG. 2 can be used to determine the susceptibility of multiple inherited genetic disorders that can develop in offspring. The method 200 allows for screening of both individuals at a time, and provides specific recommendations, based on the binary output. The present invention provides recommendations for all possible scenarios. Hence, the present invention algorithm allows an individual to accurately check whether his or her offspring is at risk of developing any inherited genetic disorders. It should be appreciated that the method 200 can also be used for diagnosis and treatment of various diseases, which are clinically actionable by a person skilled in the art.

### Example 4

The steps depicted in the flowchart of FIGS. 2A and 2B can also be explained by using the following example.

An individual suffering from a genetic disorder is advised to undergo a genetic test. The test is done by collecting genetic samples from the individual. The genetic samples are collected by extracting a DNA sample from saliva, blood, and the like. A plurality of SNVs is identified by performing a genotyping assay on the genetic sample. The plurality of SNVs is compared against the database of SNV markers associated with a specific demographic segment. Following the analysis, risk SNVs or genes are identified, which correspond to genetic disorder Type I diabetes mellitus. The identified risk genes include TCERG1L as SNV#101, TMEM163 as SNV#102 and TGFBR3 as SNV#103. A profile of the individual with such risk genes is created in the database. Each SNV in the profile is analyzed against the database of drug responses to retrieve each drug response and its corresponding recommendations. An individual-specific matrix is generated by using all SNVs with complete coverage of data.

The individual is advised by a doctor or physician to use a drug called Symlin. However, the individual wants to cross-check the recommended drug response over the risk genes, in order to avoid adverse consequences. Therefore, the individual enters the name “Symlin” through an input device, which is then stored in the database. The individual also enters demographic data such as his or her family name, Jamuar, from Patna. The demographic data is stored in the profile of the individual. The system checks the drug response of “Symlin” on the risk genes by comparing these with the generated matrix. Based on the comparison, the individual receives a recommendation that “Symlin” has an adverse reaction on an individual's risk genes. The recommendation is stored in the profile of the individual. Another matrix is generated by using demographic data, drug responses and corresponding recommendations for the individual.

If there are 1000 individuals with the family name of Jamuar from Patna, showing an adverse reaction to “Symlin” in the matrix, the system analyzes and predicts a pattern to anticipate that any individual with the family name Jamuar from Patna has the potential for an adverse reaction to “Symlin”. If an individual without profile or genetic data keys in the family name Jamuar from Patna and the drug name Symlin, the system automatically warns the individual about the drug Symlin's potential adverse reaction. Here, the possible potential adverse reaction of the drug on the individual was already anticipated by the system.

In an embodiment, the matrices are updated regularly, based on variations in the database of drug responses. The system 100 helps the physician or doctor not to rely on a prior analysis or knowledge of genes. The recommendations are categorized and generated, based on the individual's genetic profile. In an embodiment, the recommendations can be, but are not limited to, the specified drug information, drug dosage, efficiency of the drug, adverse effects, healthcare precautions, and the like. The system 100 generates recommendations for an individual who does not have genetic data, but has similar demographic data. It should also be appreciated that the method 200 is not limited to any specific disease and can be used for diagnosis and treatment of various diseases that are clinically actionable by a person skilled in the art.

### Example 5

The steps involved in the flowchart of FIGS. 5A and B can also be explained by using the following example:

The physician of a 30-year-old Malayali in Kerala wants to be well-informed about appropriate decisions to be taken on the individual's health care. The physician is advised to use a genomic analyzer for his or her patient. The genomic analyzer enhances interpretation of the individual's genomic data. The physician enters the genome sequence as well as the phenotype data of the person in the computer system 300. The phenotype data of the individual is listed in Table A:

TABLE A

| | |
|---|---|
| Age 0 | Normal birth preferences |
| Age 0 | No extra finger |
| Age 1 | Lactose intolerance |
| Age 5-10 | Asthma as child |
| Age 15 | Diabetes |

The genomic analyzer compares the entered genomic sequence with population-related data specific to Kerala. Following the analysis, a list of associated gene variants is generated. Table B depicts the list of associated gene variants.

TABLE B

Extra Finger Mutation
Lactose Intolerance
Risk of Colon Cancer
Anaesthesia Risk

The genomic analyzer then compares the list of associated gene variants with the person's phenotype. The comparison reveals the analysis detailed in Table C.

TABLE C

| | |
|---|---|
| Extra Finger Mutation | Unconfirmed |
| Lactose Intolerance | Confirmed |
| Risk of Colon Cancer | Unconfirmed |
| Anaesthesia Risk | Unconfirmed |

Following the analysis, the list of unconfirmed phenotypes is compared again with the person's family phenotype data. The individual's family phenotype data is then entered in the genomic analyzer, as shown in Table D:

TABLE D

| Mother | Father | Sister |
|---|---|---|
| Extra Finger Mutation | Lactose Intolerance | Asthma Risk |
| at Age 0 | at Age 1 | |
| Anaesthesia Risk at | Diabetes at Age 35 | Anaesthesia Risk at |
| Age 10 | | Age 45 |

The genomic analyzer compares the person's family phenotype data with the previous analysis. The comparison confirms the anaesthesia risk, which was earlier unconfirmed. Further, the genomic analyzer compares the person's family genotype data, which reveals that he or she is at risk of developing diabetes in the future. Following the comparison and analysis, the result is displayed. Table E illustrates the updated genotype and phenotype analysis of the person.

TABLE E

| | |
|---|---|
| Extra Finger Mutation | Unconfirmed |
| Lactose Intolerance | Confirmed |
| Risk of Colon Cancer | Unconfirmed |
| Anaesthesia Risk | Anaesthesia Risk at Age 45 |
| Diabetes | Diabetes at age 35 |

The analysis is repeated, and if there is a change in output, a “revised real time” report is generated unless the individual specifies that there should be no updates or limits their number or frequency. The genomic and phenotype data is then updated to the population data specific to Malayalis in Kerala.

In this invention, the genomic analyzer relates the genomic data of an individual with that of his or her family and the population to which the individual belongs, and thereafter, analyzes this data in relation to his or her clinical presentation (ranging from asymptomatic to his or her being affected with a certain disorder). In an embodiment, the individual's data 312 is validated with the population data 310 and also with the individual's family data 314. Therefore, the predicted genotype-phenotype association data is as accurate and relevant as possible. Since the population data 310 is demography-specific, it enables a clear and relevant understanding of the genotype-phenotype association data. In another embodiment, the population data 310 is updated regularly in real time and therefore enables the individual to diagnose the disorder well in advance.

As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

What is claimed is:

1. A method for providing a preventative health recommendation, a disease diagnosis, a disease risk assessment, or a pharmacogenetic recommendation for a person, the method comprising:
receiving a genetic sample of the person;
accessing a nucleic acid sequence database, the database comprising one or more single nucleotide variant (SNV)-containing nucleic acid sequences, wherein each SNV has an association with the pathological condition in a demographic segment to which the person belongs; wherein the SNV is selected from the group consisting of the SNV of SEQ ID NOS:1-511; and wherein the SNV-containing nucleic acid sequence is at least 10 nucleotides in length and has over its length at least 70%, at least 75%, at least 80%, at least 85%, at least 90%, at least 95%, at least 96%, at least 97%, at least 98%, at least 99% or 100% contiguous sequence identity to a sequence selected from the group consisting of SEQ ID NO:1-511;
performing a genotyping assay on the genetic sample to identify an SNV marker in the genetic sample of the person;
identifying a risk SNV in the genetic sample of the person by comparing the SNVs in the genetic sample of the person with the database of SNV markers; and
providing a diagnosis of a pathological condition in the person based on the identification of risk SNVs in the genetic sample of the person.

2. The method of claim 1, wherein the database comprises at least 10, 20, 30, 40, 50, 60, 70, 80 or 90 or more SNV-containing nucleic acid sequences.

3. The method of claim 1, wherein the database comprises at least 100, 200, 300, 400 or 500 or more SNV-containing nucleic acid sequences.

4. The method of claim 1, wherein the SNV-containing nucleic acid sequences are 15, 20, 25, 30, 35, 40, 45, 50, 55, 60, 65, 70, 75, 80, 85, 90, 95, or 100 nucleotides or greater in length.

5. The method of claim 1, wherein the data base consists of SNV-containing nucleic acids associated with a pathological condition that is selected from the group consisting of cancer, diseases of the eye, cardiometabolic diseases, inherited diseases, pediatric diseases, and pharmacogenetic responses to pathological conditions.

6. The method of claim 5, wherein the cancer is selected from the group consisting of breast cancer, ovarian cancer, and colon cancer.

7. The method of claim 5, wherein the disease of the eye is glaucoma or age-related macular degeneration (AMD).

8. The method of claim 5, wherein the cardiometabolic disease is selected from the group consisting of arrhythmia, e.g., long QT syndrome; clotting factor disorders, including drug response to warfarin; cardiomyopathy; coronary artery disease; cardiovascular disease, optionally associated with diabetes types I or II; hypertension; obesity; lipid disorders, such as high cholesterol, LDL, or triglycerides, or low levels of HDL, including drug response to statins; diabetes types I and II; maturity onset diabetes of the young (MODY); diabetes-associated retinopathy, obesity, enhanced waist circumference, and other complications such as neuropathy, nephropathy, foot damage, cardiovascular disease and stroke.

9. The method of claim 5, wherein the inherited disease or pediatric disease is selected from the group consisting of cystic fibrosis, congenital obstruction of the vas deferens, phenylketonuria, dopa response dystonia, epilepsy, homocystinuria, tyrosinemia, sickle cell anemia, thalassemia, Wilson's disease, non-ketotic hyperglycinemia (NKHG), glucose 6-phosphate dehydrogenase (G6PD) deficiency; maple syrup urine disease (MSUD); and congenital adrenal hyperplasia.

10. The method of claim 1, wherein the pathological condition is selected from the group consisting of cancer, diseases of the eye, cardiometabolic diseases, inherited diseases, pediatric diseases, and pharmacogenetic responses to pathological conditions.

11. The method of claim 10, wherein the cancer is selected from the group consisting of breast cancer, ovarian cancer, and colon cancer.

12. The method of claim 10, wherein the disease of the eye is glaucoma or age-related macular degeneration (AMD).

13. The method of claim 10, wherein the cardiometabolic disease is selected from the group consisting of arrhythmia, e.g., long QT syndrome; clotting factor disorders, including drug response to warfarin; cardiomyopathy; coronary artery disease; cardiovascular disease, optionally associated with diabetes types I or II; hypertension; obesity; lipid disorders, such as high cholesterol, LDL, or triglycerides, or low levels of HDL, including drug response to statins; diabetes types I and II; maturity onset diabetes of the young (MODY); diabetes-associated retinopathy, obesity, enhanced waist circumference, and other complications such as neuropathy, nephropathy, foot damage, cardiovascular disease and stroke.

14. The method of claim 10, wherein the inherited disease or pediatric disease is selected from the group consisting of cystic fibrosis, congenital obstruction of the vas deferens, phenylketonuria, dopa response dystonia, epilepsy, homocystinuria, tyrosinemia, sickle cell anemia, thalassemia, Wilson's disease, non-ketotic hyperglycinemia (NKHG), glucose 6-phosphate dehydrogenase (G6PD) deficiency; maple syrup urine disease (MSUD); and congenital adrenal hyperplasia.

15. The method of claim 1 further comprising providing a preventive healthcare recommendation to the person.

16. The method of claim 1, wherein the demographic segment is residents of India.

17. A microarray of nucleic acids, the microarray comprising one or more single nucleotide variant (SNV)-containing nucleic acid sequences; wherein the SNV is selected from the group consisting of the SNV of SEQ ID NOS:1-511; and wherein the SNV-containing nucleic acid sequences are at least 10 nucleotides in length and have over their length at least 70%, at least 75%, at least 80%, at least 85%, at least 90%, at least 95%, at least 96%, at least 97%, at least 98%, at least 99% or 100% contiguous sequence identity to a sequence selected from the group consisting of SEQ ID NO:1-511.

18. The microarray of claim 17, wherein the microarray comprises at least 10, 20, 30, 40, 50, 60, 70, 80 or 90 SNV-containing nucleic acid sequences.

19. The microarray of claim 17, wherein the microarray comprises at least 100, 200, 300, 400 or 500 SNV-containing nucleic acid sequences.

20. The microarray of claim 17, wherein the microarray comprises 511 SNV-containing nucleic acid sequences.

21. The microarray of claim 17, wherein the SNV-containing nucleic acid sequences are 15, 20, 25, 30, 35, 40, 45, 50, 55, 60, 65, 70, 75, 80, 85, 90, 95, or 100 nucleotides or greater in length.

22. The microarray of claim 17, wherein the data base consists of SNV-containing nucleic acids associated with a pathological condition is selected from the group consisting of cancer, diseases of the eye, cardiometabolic diseases, inherited diseases, pediatric diseases, and pharmacogenetic responses to pathological conditions.

23. The microarray of claim 22, wherein the cancer is selected from the group consisting of breast cancer, ovarian cancer, and colon cancer.

24. The microarray of claim 22, wherein the disease of the eye is glaucoma or age-related macular degeneration (AMD).

25. The microarray of claim 22, wherein the cardiometabolic disease is selected from the group consisting of arrhythmia, e.g., long QT syndrome; clotting factor disorders, including drug response to warfarin; cardiomyopathy; coronary artery disease; cardiovascular disease, optionally associated with diabetes types I or II; hypertension; obesity; lipid disorders, such as high cholesterol, LDL, or triglycerides, or low levels of HDL, including drug response to statins; diabetes types I and II; maturity onset diabetes of the young (MODY); diabetes-associated retinopathy, obesity, enhanced waist circumference, and other complications such as neuropathy, nephropathy, foot damage, cardiovascular disease and stroke.

26. The microarray of claim 22, wherein the inherited disease or pediatric disease is selected from the group consisting of cystic fibrosis, congenital obstruction of the vas deferens, phenylketonuria, dopa response dystonia, epilepsy, homocystinuria, tyrosinemia, sickle cell anemia, thalassemia, Wilson's disease, non-ketotic hyperglycinemia (NKHG), glucose 6-phosphate dehydrogenase (G6PD) deficiency; maple syrup urine disease (MSUD); and congenital adrenal hyperplasia.

27. A kit comprising the microarray of claim 17.

28. A kit comprising PCR primers, the primers hybridizing to one or more single nucleotide variant (SNV)-containing nucleic acid sequences for amplification of an SNV; wherein the SNV is selected from the group consisting of the SNV of SEQ ID NOS:1-511; and wherein the SNV-containing nucleic acid sequences are at least 10 nucleotides in length and have over their length at least 70%, at least 75%, at least 80%, at least 85%, at least 90%, at least 95%, at least 96%, at least 97%, at least 98%, at least 99% or 100% contiguous sequence identity to a sequence selected from the group consisting of SEQ ID NO:1-511.

29. A system for providing a preventative health recommendation, a disease diagnosis, a disease risk assessment, or a pharmacogenetic recommendation for a person, each having a risk SNV associated therewith, the risk SNV provided with a weighted score based on an odds ratio corresponding to each risk SNV, the system comprising:
an input device for receiving a genetic sample of the person;
a database comprising one or more single nucleotide variant (SNV)-containing nucleic acid sequences, wherein each SNV has an association with the pathological condition in a demographic segment to which the person belongs; wherein the SNV is selected from the group consisting of the SNV of SEQ ID NOS:1-511; and wherein the SNV-containing nucleic acid sequence is at least 10 nucleotides in length and has over its length at least 70%, at least 75%, at least 80%, at least 85%, at least 90%, at least 95%, at least 96%, at least 97%, at least 98%, at least 99% or 100% contiguous sequence identity to a sequence selected from the group consisting of SEQ ID NO:1-511
a DNA diagnostic chip in communication with the database of SNV markers, the DNA diagnostic chip configured to perform a genotyping assay on the genetic sample for identifying a plurality of SNVs, compare the plurality of SNVs with the database of SNVs for identifying a plurality of risk SNVs, and calculating a genetic risk factor for each risk SNV of the plurality of risk SNVs using the corresponding weighted score;
a comparison module for comparing the genetic risk factor and a plurality of set of ranges, the set of ranges representing the risk level of a disease on the person; and
an output device configured to provide a risk level of the set of risk levels for the person based on the comparison of the genetic risk factor with the set of ranges.
